(19) United States
(12) Patent Application Publication
(10) Pub. No.: US 2026/0334873 A1
(43) Pub. Date: Oct. 29, 2026

(54) BRAIN-INJURY TREATMENT-RESPONSE MONITORING SYSTEM

(71) Applicants: Bradley Kolls, Durham, NC (US); Chandra Throckmorton, Durham, NC (US); Leslie Collins, Durham, NC (US)

(72) Inventors: Bradley Kolls, Durham, NC (US); Chandra Throckmorton, Durham, NC (US); Leslie Collins, Durham, NC (US)

(21) Appl. No.: 19/732,663

(22) Filed: Jul. 7, 2026

Related U.S. Application Data
(63) Continuation-in-part of application No. 17/260,094, filed on Jan. 13, 2021.

(51) Int. Cl. A61B 5/00; A61B 5/31; A61B 5/369; G16H 50/70

(52) U.S. Cl. CPC A61B 5/4848; A61B 5/31; A61B 5/369; A61B 5/7221; G16H 50/70

(57) ABSTRACT

Systems and methods for brain-injury treatment-response monitoring are disclosed. A monitoring system receives continuous electroencephalogram (EEG) waveform data from a patient during a treatment period following a brain injury and receives time-stamped clinical-event data identifying a treatment event. The system defines pre-event and post-event temporal windows relative to the treatment event, transforms EEG waveform samples from the temporal windows into brain-state feature vectors, and applies a signal-quality gate to determine whether the feature vectors satisfy reliability criteria. A matched brain-injury cohort may be selected based on patient-matching parameters including injury type, injury severity, time from injury onset, and treatment-event type. The system compares a treatment-response vector derived from the feature vectors to an expected response envelope generated from the matched cohort and classifies the patient response as improvement, deterioration, plateau, deviation, or indeterminate. A treatment-management output is generated for a care team.

### CROSS-REFERENCE TO RELATED APPLICATIONS

This application contains subject matter which is related to the subject matter of the following co-pending applications. The below-listed applications are hereby incorporated herein by reference in their entirety:
This is a U.S. non-provisional application that is a continuation-in-part of a U.S. non-provisional application, Ser. No.17/260,094, inventor Bradley Kolls et al., entitled “BRAIN INJURY MONITORING WITH RECOVERY TRAJECTORY”, filed Jan. 13, 2021, which is the U.S. national stage entry, under 35 U.S.C. § 371, of International Application Number PCT/US 2019/042370, filed Jul. 18, 2019, which claims the benefit of U.S. Provisional Application No. 62/701,346, filed Jul. 20, 2018, entitled “BRAIN INJURY MONITORING DEVICE”.

### TECHNICAL FIELD OF THE INVENTION

This invention relates to systems and methods for monitoring neurological recovery following brain injury, and particularly to brain-injury treatment-response monitoring systems configured to process continuous neurophysiological waveform data, clinical-event data, and matched-cohort recovery data to generate event-anchored, quality-gated treatment-response outputs during a treatment period following a brain injury.

More particularly, the invention relates to bedside and networked monitoring systems that receive continuous electroencephalogram (EEG) waveform data from a brain-injury patient, transform the EEG waveform data into brain-state feature vectors or brain-state signatures, temporally align the brain-state information with treatment events, compare patient-specific brain-state changes to expected response information derived from matched brain-injury cohorts, and generate recovery-state, treatment-response, confidence, deviation, monitoring-control, or care-team outputs.

### BACKGROUND OF THE INVENTION

Before our invention, management of patients following brain injury often relied on intermittent neurological examinations, vital-sign monitoring, occasional imaging studies, and clinician interpretation of complex physiologic signals. Brain injuries, including traumatic brain injury, ischemic stroke, hemorrhagic stroke, hypoxic-ischemic injury, subarachnoid hemorrhage, and other acute neurologic injuries, can evolve over minutes, hours, and days. During that period, a patient may improve, deteriorate, plateau, or deviate from an expected recovery path. However, conventional monitoring practices may not provide a continuous and objective indication of how the injured brain is changing during treatment.

Prior approaches frequently rely on indirect physiologic measurements, such as blood pressure, heart rate, respiratory rate, oxygen saturation, and temperature. While these measurements can be clinically useful, they do not directly characterize brain function. A patient's vital signs may appear acceptable while the patient's neurological condition is changing in a clinically significant way. Conversely, changes in systemic physiology may be difficult to interpret without a corresponding indication of how the brain is responding. As a result, clinicians may lack a direct and continuous measure of brain recovery during the acute and subacute treatment period.

Prior approaches also rely heavily on intermittent clinical examinations. Neurological examinations can be subjective, can vary among examiners, and may be limited by sedation, intubation, impaired consciousness, aphasia, paralysis, or other patient-specific factors. In many brain-injury patients, a meaningful examination may not be available at the time when treatment decisions are being made. Even when examinations are available, they may provide only periodic snapshots rather than a continuous indication of recovery or deterioration.

Imaging-based approaches can provide important structural or perfusion information, but imaging studies are typically obtained at discrete time points. Imaging may be expensive, logistically difficult, unavailable at the bedside, and challenging to repeat frequently in critically ill patients. Imaging may also lag behind functional changes in the brain. Thus, imaging alone may not provide a practical continuous monitoring tool for evaluating whether a patient's brain function is improving or deteriorating after a treatment event.

Electroencephalogram monitoring can provide continuous information about brain activity, but prior approaches often require specialized interpretation of raw EEG waveforms or focus on limited use cases such as seizure detection. Raw EEG data can be voluminous, noisy, artifact-prone, and difficult for non-specialist bedside providers to interpret. Changes in EEG features may occur over time in ways that are difficult to detect by visual review alone, especially when clinicians must also account for medication effects, sedation, movement artifact, missing channels, electrode impedance, and other confounding conditions.

Prior approaches may also fail to relate changes in brain activity to specific treatment events. A patient may receive medication, ventilation adjustment, blood-pressure treatment, intracranial-pressure intervention, stroke intervention, seizure treatment, sedation change, rehabilitation therapy, or another clinical intervention, but conventional monitoring may not objectively determine whether a subsequent change in brain-state information represents an expected response, an unexpected response, deterioration, plateau, or insufficiently reliable data. Without event anchoring, clinicians may be left to infer whether observed changes are treatment-related, disease-related, artifact-related, or unrelated.

Prior approaches can further suffer from a lack of patient-specific comparison. Broad population averages or generic recovery expectations may not account for injury type, initial severity, time from onset, imaging pattern, medication state, sedation state, physiological condition, or treatment-event type. A recovery pattern that is concerning for one patient may be expected for another. Without a meaningful patient-specific comparator, a displayed trajectory or score may be difficult to interpret and may not provide enough context for treatment management.

Additionally, prior approaches may provide outputs without adequately communicating reliability. A trend, score, or displayed value may appear precise even when underlying signals are incomplete, noisy, artifact-contaminated, or confounded by sedation or medication state. This can create a risk that clinicians will overinterpret a generated output or disregard useful information because the basis for the output is unclear. A monitoring system that does not identify signal-quality limitations, confidence limitations, and contributing data sources may have limited usefulness in the dynamic environment of brain-injury care.

Prior approaches may also impose burdens on computing, storage, and network resources. Continuous EEG waveform data can be large and difficult to process repeatedly. Systems that transmit or reprocess raw waveform data without generating reusable intermediate representations can introduce latency, bandwidth demands, and privacy concerns. These limitations can impair practical bedside deployment and can make it difficult to integrate continuous brain monitoring into a clinical workflow.

The present invention addresses these and other shortcomings by providing a brain-injury treatment-response monitoring system for managing recovery of a patient during a treatment period following a brain injury. For these reasons and shortcomings, as well as other reasons and shortcomings, there is a long-felt need that gives rise to the present invention.

### SUMMARY OF THE INVENTION

The shortcomings of the prior approaches are overcome, and additional advantages are provided through the provision of a brain-injury treatment-response monitoring system for managing recovery of a patient during a treatment period following a brain injury. The system may include a neurophysiological sensor interface configured to receive continuous EEG waveform data, a clinical-event interface configured to receive time-stamped treatment-event data, a memory storing a rolling waveform buffer, a brain-state feature table, and matched-cohort recovery data, an output interface, and one or more processors.

In an exemplary embodiment, the one or more processors may time-align a treatment event with the continuous EEG waveform data, define pre-event and post-event temporal windows relative to the treatment event, and transform EEG waveform samples from the temporal windows into corresponding brain-state feature vectors stored in the brain-state feature table. The one or more processors may apply a signal-quality gate and may prevent classification when either feature vector fails a reliability threshold.

The one or more processors may select a matched brain-injury cohort based on patient-matching parameters including injury type, initial injury severity, time from injury onset, and treatment-event type. The one or more processors may generate an expected intervention-response envelope, generate a treatment-response vector representing a change between the pre-event and post-event brain-state feature vectors, compare the treatment-response vector to the expected intervention-response envelope, and classify the patient response as improvement, deterioration, plateau, deviation, or indeterminate.

The system may generate a treatment-management output identifying the treatment event, the treatment-response state, and a confidence indicator. In some embodiments, the comparison may be performed using the stored brain-state feature vectors without reprocessing the EEG waveform samples after the feature vectors are stored, thereby reducing repeated raw-waveform processing and supporting event-anchored, quality-gated brain-injury recovery monitoring.

Additional shortcomings of the prior approaches are overcome, and additional advantages are provided through the provision of a brain-injury treatment-response monitoring system for managing recovery of a patient during a treatment period following a brain injury. The system may include a neurophysiological sensor interface configured to receive continuous EEG waveform data, a physiological sensor interface configured to receive non-EEG physiological data, a clinical-event interface configured to receive time-stamped treatment-event data, a clinical data interface configured to receive clinical context data, a memory storing a monitoring protocol table, and one or more processors.

In an exemplary embodiment, the monitoring protocol table may associate a plurality of monitoring modes with different acquisition parameters and processing parameters. The one or more processors may generate current and previous brain-state vectors from EEG waveform data over respective temporal windows, generate a recovery-trend value based on a change between the brain-state vectors, and generate a confidence value based on the non-EEG physiological data and clinical context data.

The one or more processors may compare the recovery-trend value to an expected recovery corridor and select a monitoring mode based on the comparison, the confidence value, and a treatment-event type. The system may generate and apply a control command that changes an acquisition parameter or processing parameter of the monitoring system before a subsequent brain-state vector is generated. A treatment-response output may then be generated after system operation has been changed, thereby allowing the monitoring system to adapt its own operation during brain-injury recovery.

Additional shortcomings of the prior approaches are overcome, and additional advantages are provided through the provision of a brain-injury treatment-response monitoring system for managing recovery of a patient during a treatment period following a brain injury. The system may include a bedside monitoring apparatus, a remote cohort server, a network interface, one or more local processors, and one or more server processors.

In an exemplary embodiment, the bedside monitoring apparatus may receive and locally store continuous EEG waveform data from the patient. The one or more local processors may transform the EEG waveform data into a compressed brain-state signature comprising a time-indexed sequence of EEG-derived feature vectors and may associate the compressed brain-state signature with treatment-event metadata.

The compressed brain-state signature and treatment-event metadata may be transmitted to the remote cohort server without transmitting the continuous EEG waveform data. The remote cohort server may select a matched brain-injury cohort from a cohort database, generate cohort-response data from the matched brain-injury cohort, and transmit the cohort-response data back to the bedside monitoring apparatus.

The bedside monitoring apparatus may classify a treatment-response state using the cohort-response data and the compressed brain-state signature and may generate a treatment-response output through a local output interface. The continuous EEG waveform data may remain stored locally while the compressed brain-state signature is transmitted remotely, thereby reducing raw-waveform transmission while supporting matched-cohort treatment-response monitoring.

Additional features and advantages are realized through the techniques of the present invention. Other embodiments and aspects of the invention are described in detail herein and are considered a part of the claimed invention. For a better understanding of the invention with advantages and features, refer to the description and to the drawings.

### BRIEF DESCRIPTION OF THE FIGURES

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings, in which:

FIG. 1 illustrates one example of a brain-injury treatment-response monitoring system including an EEG sensor, a bedside monitor, an electronic health record system, a treatment-event input, a matched-cohort database, and a care-team output.

FIG. 2 illustrates one example of a brain-injury monitoring system including the electronic processing device of FIG. 8 in communication with one or more biometric sensors and one or more databases.

FIG. 3 illustrates one example of a method for determining and displaying a recovery trajectory of a patient based on patient-specific clinical information and biometric data associated with a brain injury.

FIG. 4 illustrates one example of a power analysis table including frequency-based electroencephalogram (EEG) power features extracted following varied periods of ischemia.

FIG. 5 illustrates one example of a machine-learning approach for determining a brain-injury recovery assessment using multiple patient-data streams, including text data, imaging data, and biosignal data.

FIG. 6 illustrates one example of an unweighted classifier output table generated from a plurality of patient-data classifiers, including classifiers associated with text or clinical-note data, imaging data, EEG data, ECG or heart-rate data, arterial-line or blood-pressure data, intracranial-pressure data, cuff-pressure data, laboratory-value data, medication or pharmacy-event data, ventilation or oxygenation data, and treatment-event data.

FIG. 7 illustrates one example of a weighted classifier output table generated from the classifier outputs of FIG. 6, wherein weighting factors are applied to respective classifier outputs to generate weighted classification outputs and a weighted aggregate output usable for injury-severity classification, brain-state classification, recovery-trajectory generation, treatment-response analysis, or deviation detection.

FIG. 8 illustrates one example of an electronic processing device configured for receiving, processing, storing, and displaying patient information associated with brain-injury monitoring.

FIG. 9 illustrates one example of a treatment-response timeline showing an injury event, a baseline window, a treatment event, a post-treatment window, an expected response envelope, an actual response, and a deviation flag.

FIG. 10 illustrates one example of a flowchart for generating a temporally anchored brain-state signature based on continuous EEG waveform data and time-stamped treatment-event data.

FIG. 11 illustrates one example of a matched brain-injury cohort selection engine configured to select prior brain-injury patients based on patient-matching parameters.

FIG. 12 illustrates one example of a treatment-response classification workflow configured to classify a treatment-response state as improvement, deterioration, plateau, deviation, or indeterminate.

FIG. 13 illustrates one example of a clinical dashboard showing a recovery state, an intervention response, a confidence level, contributing signals, and a treatment-management output.

The detailed description explains the preferred embodiments of the invention, together with advantages and features, by way of example with reference to the drawings.

### DETAILED DESCRIPTION OF THE INVENTION

Brain injuries, including traumatic brain injury, ischemic stroke, hemorrhagic stroke, hypoxic-ischemic injury, subarachnoid hemorrhage, and other acute neurologic injuries, can evolve over minutes, hours, and days following an initial injury event. During this period, a patient's neurologic condition may improve, deteriorate, plateau, or deviate from an expected recovery path in response to treatment, disease progression, secondary injury, medication effects, physiologic instability, or other clinical factors. Existing clinical management is often driven by intermittent neurologic examinations, vital-sign thresholds, imaging studies obtained at discrete times, and clinician interpretation of complex physiologic signals. These approaches may fail to provide a continuous, objective, and patient-specific indication of how the injured brain is responding to treatment over time.

Continuous electroencephalogram (EEG) monitoring can provide a direct and ongoing measure of brain function, but raw EEG waveform data is difficult to interpret at the bedside and often requires specialized review. In many clinical environments, EEG data is used primarily for seizure detection or retrospective analysis rather than as a continuous treatment-response signal that can be integrated with clinical events, patient context, and prior recovery patterns. Raw EEG waveform data may also be voluminous, noisy, artifact-prone, and influenced by medication state, sedation state, missing channels, motion, muscle artifact, and other confounders. As a result, even when EEG data is available, it may not be converted into a reliable treatment-management output that identifies whether a patient is improving, deteriorating, plateauing, deviating from expected recovery, or producing insufficiently reliable data for classification.

The present disclosure relates to brain-injury treatment-response monitoring systems and methods for managing recovery of a patient during a treatment period following a brain injury. In some implementations, the system receives continuous EEG waveform data from one or more sensors coupled to the patient and receives time-stamped clinical-event data identifying one or more treatment events performed during the treatment period. The treatment event may include, for example, administration of medication, a seizure-related intervention, a blood-pressure intervention, a ventilation change, an intracranial-pressure intervention, a stroke intervention, a sedation change, a rehabilitation event, a neurologic stimulation event, or another clinically relevant event. By anchoring EEG-derived analytic measures to the timing of the treatment event, the system can evaluate how the patient's brain state changes before and after the event rather than merely displaying an unanchored trend.

In some implementations, the system defines a pre-event temporal window and a post-event temporal window relative to an event time associated with the treatment event. EEG waveform samples in the pre-event temporal window are transformed into a pre-event brain-state feature vector, and EEG waveform samples in the post-event temporal window are transformed into a post-event brain-state feature vector. The feature vectors may include EEG-derived features such as spectral power, interhemispheric asymmetry, burst-suppression burden, seizure burden, coherence, entropy, rhythmicity, amplitude variability, stimulation reactivity, or other features derived from the waveform data. The system may store the feature vectors in a brain-state feature table so that subsequent comparisons and treatment-response classifications can be performed using the stored feature-vector representation without requiring repeated direct reprocessing of the raw EEG waveform samples.

The system may also receive clinical information associated with the patient, including injury type, initial injury severity, time from injury onset, neurological examination findings, imaging findings, medication state, sedation state, intracranial pressure, arterial pressure, electrocardiogram data, laboratory values, patient demographics, prior history, and other clinical context. In some implementations, the system uses this information to select a matched brain-injury cohort from prior-patient recovery data. The matched cohort may be selected based on patient-matching parameters such as injury type, severity, age, imaging pattern, treatment-event type, time from injury onset, physiologic state, medication state, or monitoring window. The matched cohort may then be used to generate an expected intervention-response envelope for the treatment event.

The patient's actual treatment response may be represented by a treatment-response vector indicating a change from the pre-event brain-state feature vector to the post-event brain-state feature vector. The system compares the treatment-response vector to the expected intervention-response envelope generated from the matched brain-injury cohort. Based on the comparison, the system may classify a treatment-response state of the patient as improvement, deterioration, plateau, deviation, or indeterminate. In some implementations, the classification is conditioned on a signal-quality gate that evaluates whether the underlying signals and clinical context satisfy a reliability threshold. The signal-quality gate may prevent classification when the EEG data or associated patient data is unreliable due to artifact, missing channels, signal saturation, electrode impedance, motion, muscle activity, sedation-related confounding, insufficient cohort match, or other reliability limitations.

The treatment-response monitoring system may generate a treatment-management output for presentation to a care team. The treatment-management output may identify the treatment event, the treatment-response state, a confidence indicator, contributing signals, deviation metrics, response plots, matched-cohort information, monitoring recommendations, or other information useful for clinical management. In some implementations, the output may indicate that the patient is responding better than expected, worse than expected, within an expected recovery range, or indeterminate due to insufficient reliability. The system may thereby convert complex waveform and clinical data into a structured, event-anchored, quality-gated treatment-response output.

In some implementations, the system may adapt operation of the monitoring apparatus based on recovery trends and confidence values. For example, the system may increase sampling rate, adjust an EEG channel set, change an electrode montage, increase feature-extraction frequency, change a classifier-update interval, modify alert thresholds, or increase data-transmission frequency when the patient deviates from an expected recovery corridor or when confidence criteria are satisfied. Conversely, the system may reduce monitoring intensity or data-transmission frequency when the patient remains within an expected recovery corridor for a stability period. In this manner, the system may improve operation of the monitoring apparatus itself by changing acquisition or processing parameters in response to the patient's brain-state trend and the reliability of the data.

In some implementations, the system may include a bedside monitoring apparatus and a remote cohort server. The bedside monitoring apparatus may locally store continuous EEG waveform data and transform the waveform data into a compressed brain-state signature. The compressed brain-state signature may include a time-indexed sequence of EEG-derived feature vectors, treatment-event metadata, signal-quality flags, artifact flags, medication-state flags, sedation-state flags, and other context. The compressed brain-state signature may be transmitted to the remote cohort server without transmitting the continuous raw EEG waveform data. The remote cohort server may use the compressed brain-state signature to select a matched cohort and return cohort-response data to the bedside monitoring apparatus. The bedside monitoring apparatus may then classify the treatment-response state using the cohort-response data while maintaining the raw EEG waveform data locally. This distributed architecture may reduce network burden, support privacy-preserving operation, and enable bedside classification even when communication with the remote cohort server is intermittent.

The disclosed systems and methods therefore provide a technical framework for converting continuous EEG waveform data and clinical-event data into temporally anchored brain-state signatures, matched-cohort comparisons, quality-gated treatment-response classifications, adaptive monitoring-control operations, and care-team outputs. Rather than merely calculating or displaying a recovery trajectory, the system processes continuous neurophysiological waveform data in relation to time-stamped clinical events and matched recovery data to determine how a brain-injury patient is responding during a treatment period. The resulting system may support earlier detection of improvement or deterioration, more objective assessment of intervention effectiveness, more reliable monitoring of brain-injury recovery, and improved operation of bedside monitoring technology.

### Definitions

In the present invention, the term “brain injury” is intended to mean an injury, disease event, ischemic event, hemorrhagic event, traumatic event, hypoxic event, neurologic insult, or other condition affecting brain structure, brain perfusion, brain electrical activity, brain function, or neurological recovery. The term may include traumatic brain injury, ischemic stroke, hemorrhagic stroke, hypoxic-ischemic injury, subarachnoid hemorrhage, diffuse axonal injury, contusion, seizure-related injury, post-operative brain injury, and other acute or subacute neurological injuries.

In the present invention, the term “treatment period” is intended to mean a period during which a patient is monitored following a brain injury and may include an acute period, an intensive-care period, an inpatient period, a rehabilitation period, a post-procedure period, a post-medication period, or another monitoring period during which recovery, deterioration, plateau, deviation, or treatment response may be evaluated.

In the present invention, the term “treatment event” is intended to mean a time-identifiable clinical event, intervention, medication administration, procedure, therapy, physiologic event, monitoring event, or contextual event that can be used as a temporal anchor for evaluating patient response. A treatment event may include an anti-seizure medication, sedative medication, osmotic therapy, blood-pressure intervention, ventilation adjustment, intracranial-pressure intervention, thrombolytic intervention, thrombectomy intervention, rehabilitation intervention, neurological stimulation event, imaging event, alarm event, clinical examination event, or other event relevant to brain-injury monitoring.

In the present invention, the term “clinical-event data” is intended to mean data identifying or describing one or more treatment events, including an event type, event time, event duration, event dose, event source, event status, event confidence, or event-related metadata. Clinical-event data may be manually entered, automatically received, extracted from an electronic health record, received from a bedside device, or derived from another clinical system.

In the present invention, the term “event time” is intended to mean a time associated with a treatment event and may include a start time, stop time, administration time, intervention time, procedure time, detection time, onset time, documentation time, or estimated time used to align patient data relative to the treatment event.

In the present invention, the term “time-align” is intended to mean associating, synchronizing, indexing, registering, or otherwise relating patient data to an event time, injury time, monitoring time, clock time, relative time, or other temporal reference.

In the present invention, the term “pre-event temporal window” is intended to mean a time interval before, overlapping with, or otherwise preceding a treatment event that is used to characterize a patient state before or at the beginning of the treatment event. The pre-event temporal window may be fixed, variable, clinician-selected, automatically selected, treatment-specific, injury-specific, or dynamically adjusted.

In the present invention, the term “post-event temporal window” is intended to mean a time interval after, overlapping with, or otherwise following a treatment event that is used to characterize a patient state after or during the treatment event. The post-event temporal window may be fixed, variable, clinician-selected, automatically selected, treatment-specific, injury-specific, or dynamically adjusted.

In the present invention, the term “continuous EEG waveform data” is intended to mean EEG signal data acquired over a monitoring period and may include raw EEG waveform samples, filtered EEG waveform samples, segmented EEG waveform samples, multi-channel EEG data, single-channel EEG data, montage-derived EEG data, time-stamped EEG data, or other EEG-derived time-series signal data. The term “continuous” does not require uninterrupted or gap-free data and may include data having missing portions, artifacts, intermittent acquisition, periodic sampling, or event-triggered acquisition.

In the present invention, the term “neurophysiological sensor interface” is intended to mean hardware, software, firmware, circuitry, a port, a communication module, an application programming interface, a receiver, a transceiver, or another interface configured to receive neurophysiological data from one or more sensors, sensor systems, monitoring devices, or data sources.

In the present invention, the term “physiological sensor interface” is intended to mean hardware, software, firmware, circuitry, a port, a communication module, an application programming interface, a receiver, a transceiver, or another interface configured to receive non-EEG physiological data from one or more sensors, sensor systems, monitoring devices, or data sources.

In the present invention, the term “brain-state feature vector” is intended to mean a structured representation of one or more features derived from EEG waveform data and, optionally, other patient data during a time window. A brain-state feature vector may include spectral features, temporal features, spatial features, waveform features, connectivity features, asymmetry features, seizure-related features, burst-suppression features, entropy features, rhythmicity features, stimulation-reactivity features, physiologic features, clinical-context features, confidence values, or signal-quality values.

In the present invention, the term “pre-event brain-state feature vector” is intended to mean a brain-state feature vector generated using data associated with a pre-event temporal window.

In the present invention, the term “post-event brain-state feature vector” is intended to mean a brain-state feature vector generated using data associated with a post-event temporal window.

In the present invention, the term “brain-state signature” is intended to mean one or more brain-state feature vectors, a time-indexed sequence of brain-state feature vectors, a compressed representation of one or more brain-state feature vectors, or another structured representation of a patient's brain state over one or more time windows.

In the present invention, the term “compressed brain-state signature” is intended to mean a reduced, summarized, encoded, feature-based, de-identified, or otherwise transformed representation of EEG waveform data and associated context that is smaller than, less detailed than, or different from the continuous EEG waveform data from which it is derived. A compressed brain-state signature may include time-indexed EEG-derived feature vectors, treatment-event metadata, signal-quality flags, artifact flags, medication-state flags, sedation-state flags, cohort-matching parameters, or other metadata.

In the present invention, the term “brain-state feature table” is intended to mean a stored data structure containing one or more analytic measures, calculated parameters, feature vectors, brain-state signatures, signal-quality values, time-window identifiers, event anchors, or related metadata derived from patient data. The brain-state feature table may be stored in local memory, remote memory, a database, a cache, a file, or another computer-readable storage structure.

In the present invention, the term “rolling waveform buffer” is intended to mean a memory region or data structure configured to store EEG waveform data for a moving, limited, selectable, or otherwise managed time interval. A rolling waveform buffer may overwrite, archive, compress, retain, or discard waveform data according to a buffer-retention setting, monitoring mode, signal-quality condition, or clinical event.

In the present invention, the term “analytic measure” is intended to mean a calculated value, feature, parameter, score, index, ratio, statistic, transform output, classifier input, or other derived representation generated from EEG waveform data, physiological data, clinical data, imaging data, text data, or combinations thereof.

In the present invention, the term “signal-quality gate” is intended to mean one or more processing operations, rules, thresholds, models, circuits, software modules, or decision logic configured to determine whether data satisfies a reliability condition before the data is used for classification, comparison, display, alerting, storage, transmission, or treatment-response output generation. The signal-quality gate may evaluate electrode impedance, missing channels, signal saturation, motion artifact, muscle artifact, noise, data dropout, sedation-related confounding, medication-related confounding, insufficient clinical context, insufficient cohort matching, or other reliability conditions.

In the present invention, the term “reliability threshold” is intended to mean a threshold, rule, score, condition, confidence level, signal-quality requirement, data-completeness requirement, cohort-match requirement, or other criterion used to determine whether data is sufficiently reliable for a particular processing operation or output.

In the present invention, the term “treatment-response vector” is intended to mean a derived data structure representing a change between a pre-event feature vector and a post-event feature vector associated with a patient and a treatment event. The treatment-response vector may include one or more values calculated from corresponding features of the pre-event feature vector and the post-event feature vector, including a difference, ratio, normalized difference, distance, direction, probability shift, classifier-distribution shift, feature-space shift, or another measure of change across the treatment event.

In the present invention, the term “patient-matching parameters” is intended to mean one or more parameters used to select, rank, filter, weight, or compare prior patient data relative to a current patient. Patient-matching parameters may include injury type, initial injury severity, time from injury onset, treatment-event type, age, imaging findings, vascular territory, neurological examination findings, Glasgow Coma Scale score, National Institutes of Health Stroke Scale score, intracranial pressure, medication state, sedation state, physiological state, laboratory values, EEG-derived features, or brain-state signature data.

In the present invention, the term “matched-cohort recovery data” is intended to mean recovery data, outcome data, treatment-response data, brain-state feature data, patient profile data, expected response data, cohort models, or other information associated with one or more prior brain-injury patients.

In the present invention, the term “expected intervention-response envelope” is intended to mean a cohort-derived, model-derived, statistically derived, or otherwise generated representation of an expected response after a treatment event. An expected intervention-response envelope may include percentile boundaries, confidence intervals, upper and lower boundaries, recovery corridors, time-indexed boundaries, model-predicted response curves, outcome bands, or other expected-response information. This definition tracks the spec's discussion of percentile bands and expected-response boundaries.

In the present invention, the term “expected recovery corridor” is intended to mean an expected range, band, path, envelope, trajectory, region, set of boundaries, or other comparative representation of recovery for a brain-injury patient or matched cohort.

In the present invention, the term “actual response” is intended to mean a patient-specific measured, calculated, inferred, classified, or displayed response based on one or more brain-state feature vectors, treatment-response vectors, classifier outputs, recovery-trend values, EEG-derived features, physiological data, clinical data, or combinations thereof.

In the present invention, the term “treatment-response state” is intended to mean a classification, label, score, state, status, or other output indicating how a patient appears to be responding during a treatment period. A treatment-response state may include improvement, deterioration, plateau, deviation, indeterminate, within expected range, above expected range, below expected range, or another response category.

In the present invention, the term “improvement” is intended to mean a treatment-response state in which patient data indicates a favorable change, recovery-associated change, movement toward a lower severity state, movement toward an expected or better-than-expected response, or other beneficial brain-state change.

In the present invention, the term “deterioration” is intended to mean a treatment-response state in which patient data indicates an unfavorable change, worsening, movement toward a higher severity state, movement away from an expected recovery pattern, or other adverse brain-state change.

In the present invention, the term “plateau” is intended to mean a treatment-response state in which patient data indicates little change, insufficient change, stable change, substantially flat change, or no meaningful movement toward improvement or deterioration over a selected temporal window.

In the present invention, the term “deviation” is intended to mean a treatment-response state, flag, value, or condition in which a patient's actual response differs from an expected response envelope, expected recovery corridor, matched-cohort response, threshold, percentile boundary, or other comparator by an amount, direction, duration, or pattern satisfying a deviation criterion.

In the present invention, the term “indeterminate” is intended to mean a treatment-response state or output indicating that the system does not classify the patient response as improvement, deterioration, plateau, or deviation due to insufficient reliability, insufficient data, poor signal quality, insufficient cohort match, conflicting data, low confidence, or another limiting condition.

In the present invention, the term “confidence indicator” is intended to mean a displayed, stored, transmitted, or calculated value, label, score, flag, range, or status indicating confidence, uncertainty, reliability, signal quality, cohort-match quality, model certainty, or sufficiency of data associated with an output.

In the present invention, the term “treatment-management output” is intended to mean an output generated for clinical management, monitoring management, review, display, alerting, documentation, or communication. A treatment-management output may include a treatment-response state, treatment-event identifier, confidence indicator, deviation flag, escalation indication, de-escalation indication, monitoring-frequency change indication, intervention-effectiveness indication, alert, dashboard output, report, data export, notification, or other care-team-facing output.

In the present invention, the term “monitoring mode” is intended to mean an operating state, protocol, setting group, or configuration of the monitoring system. A monitoring mode may define or affect acquisition parameters, processing parameters, alert parameters, transmission parameters, storage parameters, classifier parameters, cohort-comparison parameters, or display parameters.

In the present invention, the term “acquisition parameter” is intended to mean a parameter controlling acquisition, receipt, sampling, buffering, channel selection, montage selection, or transmission of sensor data. Acquisition parameters may include sampling rate, EEG channel set, electrode montage, data-transmission frequency, waveform-buffer retention period, sensor selection, impedance-check frequency, or acquisition duration.

In the present invention, the term “processing parameter” is intended to mean a parameter controlling signal processing, feature extraction, classification, comparison, gating, alert generation, storage, or display. Processing parameters may include feature-extraction interval, classifier-update interval, alert threshold, signal-quality threshold, cohort-comparison interval, temporal-window length, artifact-rejection setting, confidence threshold, or model-selection setting.

In the present invention, the term “control command” is intended to mean a command, instruction, signal, message, software operation, parameter update, configuration update, or other control output that changes operation of the monitoring system, a sensor interface, a processor, a memory, an output interface, or another system component.

In the present invention, the term “recovery-trend value” is intended to mean a calculated value, score, vector, slope, difference, direction, index, probability shift, classifier change, or other representation of a change in brain-state information across two or more temporal windows.

In the present invention, the term “clinical context data” is intended to mean patient-related information used to interpret, classify, compare, gate, or display brain-injury monitoring data. Clinical context data may include injury type, time from injury onset, neurological examination findings, imaging findings, medication state, sedation state, intracranial pressure, arterial pressure, ECG data, laboratory values, demographics, clinical history, prior outcomes, treatment-event history, or other patient-context information.

In the present invention, the term “non-EEG physiological data” is intended to mean patient physiological data other than EEG waveform data, including intracranial pressure data, arterial pressure data, ECG data, pulse oximetry data, respiratory data, temperature data, movement data, NIRS data, laboratory-derived physiologic data, or other physiologic information.

In the present invention, the term “cohort-response data” is intended to mean data generated from, selected from, summarized from, or derived from a matched brain-injury cohort. Cohort-response data may include expected response envelopes, recovery corridors, percentile boundaries, outcome probabilities, cohort summaries, similarity scores, confidence values, or treatment-response distributions.

In the present invention, the term “care team” is intended to mean one or more physicians, nurses, advanced practice providers, technicians, therapists, remote monitors, caregivers, clinical systems, hospital systems, or other persons or systems involved in monitoring, managing, reviewing, documenting, or responding to patient care information.

In the present invention, the term “locally stored” is intended to mean stored at, in, near, or under the control of a bedside monitoring apparatus, patient-side system, local hospital system, local memory, or local computing environment, as distinguished from being transmitted as raw waveform data to a remote cohort server.

In the present invention, the term “remote cohort server” is intended to mean one or more computing devices, servers, cloud systems, hospital systems, distributed computing resources, or databases configured to store, process, select, or return cohort-related data. The term “remote” does not require geographic separation and may refer to a logical separation from the bedside monitoring apparatus.

In the present invention, the term “without transmitting the continuous EEG waveform data” is intended to mean that the system transmits a compressed brain-state signature or feature-based representation while omitting at least a portion of the raw continuous EEG waveform data from the transmission. The phrase does not exclude transmission of metadata, selected waveform excerpts, quality-control segments, derived features, alerts, timestamps, or other non-continuous-waveform information unless expressly stated.

In the present invention, the term “without reprocessing the EEG waveform samples” is intended to mean that a later comparison, classification, or output generation operation uses stored feature vectors, analytic measures, or brain-state signatures without again performing the same waveform-to-feature transformation on the same EEG waveform samples for that operation. The term does not exclude other processing of different waveform samples, quality-control processing, storage operations, visualization, audit processing, or optional reprocessing when desired.

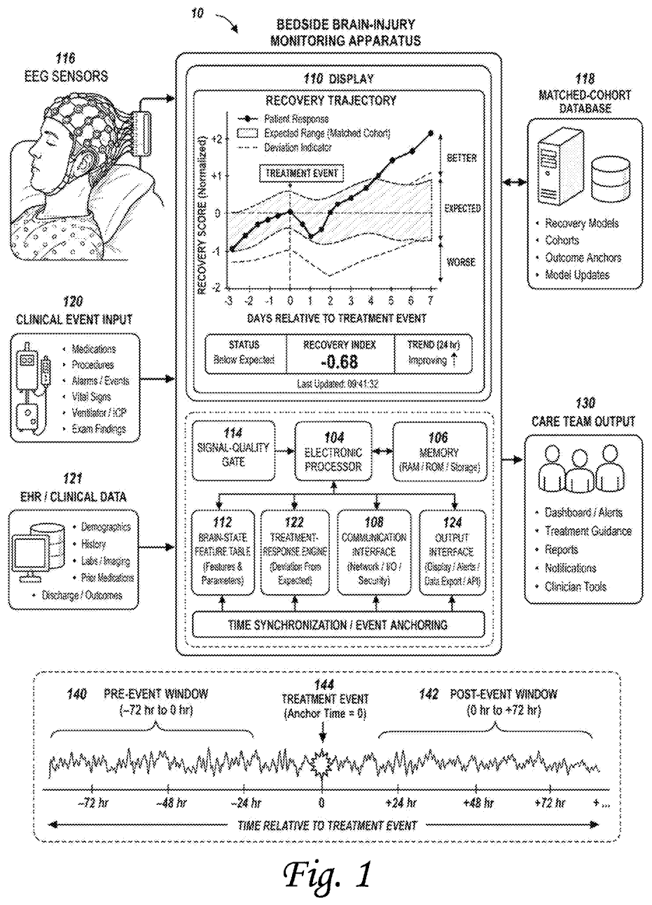

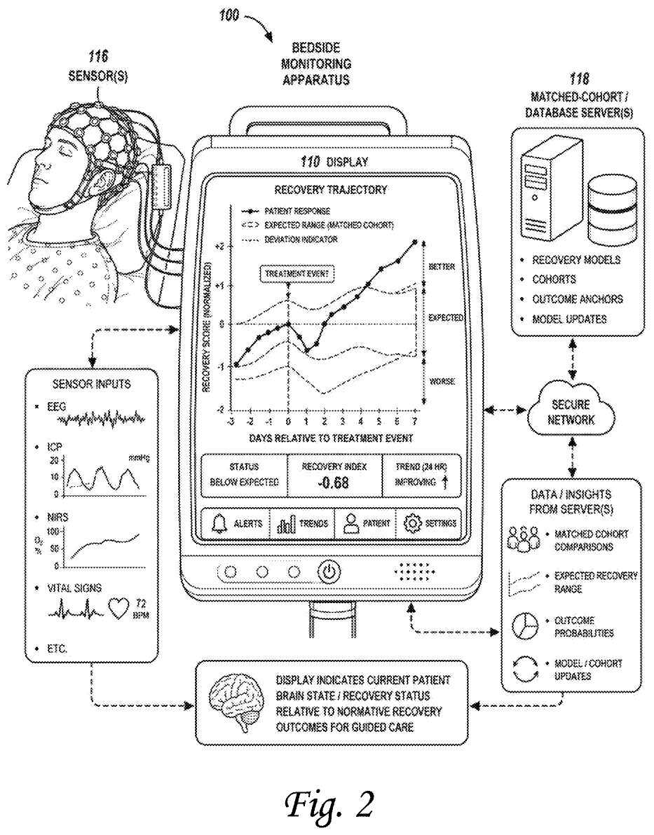

Turning now to the drawings in greater detail, it will be seen that in FIG. 1, there is illustrated one example of an overall brain-injury treatment-response monitoring system 10 including a bedside brain-injury monitoring apparatus 100 (better illustrated in FIG. 2), one or more EEG sensors 116, a clinical-event input 120, an EHR/clinical data source 121, a matched-cohort database 118, and a care-team output 130. FIG. 1 can provide a high-level overview of how patient-specific neurophysiological data, clinical-event data, and matched-cohort recovery data can be combined to generate a treatment-response output that is anchored to a treatment event rather than merely displaying an uncontextualized physiologic trend. The system 10 can thereby convert continuous brain-monitoring data into a practical clinical-management output tied to brain-injury recovery and treatment response.

In an exemplary embodiment, the EEG sensors 116 can be coupled to a patient during a treatment period following a brain injury. The EEG sensors 116 can acquire continuous EEG waveform data from the patient and can transmit the continuous EEG waveform data to the bedside brain-injury monitoring apparatus 100. Although EEG sensors 116 are shown, the sensors can include or be used with additional biometric or physiologic sensors, including intracranial pressure sensors, ECG sensors, arterial-line sensors, pulse oximetry sensors, respiratory sensors, temperature sensors, movement sensors, near-infrared spectroscopy sensors, pupillometry devices, or other patient-monitoring devices. The continuous waveform acquisition can allow the system 10 to detect changes in brain state over time, including changes that may occur before, during, or after a treatment event.

The clinical-event input 120 can provide event-related data to the bedside brain-injury monitoring apparatus 100. As shown, the clinical-event input 120 can include medications, procedures, alarms/events, vital signs, ventilator/ICP information, and exam findings. The clinical-event input 120 can be manual, automated, or a combination thereof. For example, a clinician may manually identify administration of a medication, or the system 10 may automatically receive treatment-event information from a medication-administration record, infusion pump, ventilator, bedside monitor, electronic health record, procedure log, nurse documentation system, or other clinical system. In this way, the system 10 can time-anchor patient waveform data to actual clinical events instead of requiring a clinician to retrospectively infer whether a physiologic change was related to a treatment.

The EHR/clinical data source 121 can provide clinical context data to the bedside brain-injury monitoring apparatus 100. In the illustrated example, the EHR/clinical data source 121 can include demographics, history, labs/imaging, prior medications, and discharge/outcome information. The clinical context data can also include injury type, initial injury severity, time from injury onset, neurological examination findings, imaging findings, Glasgow Coma Scale score, National Institutes of Health Stroke Scale score, vascular territory, medication state, sedation state, comorbidities, laboratory values, prior outcomes, and other patient-specific information. This clinical context can be used to interpret the patient's brain-state information in a more tailored manner than approaches that rely only on vital-sign thresholds, isolated EEG review, or broad population averages.

The matched-cohort database 118 can store or access recovery models, cohorts, outcome anchors, and model updates. In an exemplary embodiment, the matched-cohort database 118 can include recovery data for prior brain-injury patients, including de-identified patient profiles, EEG-derived features, treatment-event histories, imaging findings, clinical outcomes, and recovery trajectories. The matched-cohort database 118 can be local, remote, cloud-based, hospital-based, research-network based, or distributed across multiple systems. The matched-cohort database 118 can be used to generate an expected response range or expected intervention-response envelope for a patient having similar injury and treatment characteristics.

The bedside brain-injury monitoring apparatus 100 can include a display 110, a signal-quality gate 114, an electronic processor 104, a memory 106, a brain-state feature table 112, a treatment-response engine 122, a communication interface 108, and an output interface 124. These components can cooperate to provide a technical processing architecture for transforming raw or continuous patient data into a treatment-response classification or recovery-management output. The use of the signal-quality gate 114, the brain-state feature table 112, and the treatment-response engine 122 can help frame the system 10 as more than generic data collection and display. In particular, the bedside brain-injury monitoring apparatus 100 can perform event-aligned signal processing, reliability gating, feature-vector storage, matched-cohort comparison, and treatment-response output generation.

In an exemplary embodiment, the electronic processor 104 can receive EEG waveform data from the EEG sensors 116, clinical-event data from the clinical-event input 120, and clinical context data from the EHR/clinical data source 121. The memory 106 can store program instructions, waveform buffers, feature tables, patient records, models, event timestamps, cohort data, and generated outputs. The communication interface 108 can provide network input/output and security functions for communicating with the matched-cohort database 118, the EHR/clinical data source 121, the clinical-event input 120, and external care-team systems. The output interface 124 can generate display outputs, alerts, data exports, reports, application programming interface outputs, clinician notifications, or other outputs.

The signal-quality gate 114 can evaluate the reliability of the patient data before a treatment-response classification is generated or accepted. The signal-quality gate 114 can assess electrode impedance, missing EEG channels, signal saturation, motion artifact, muscle artifact, excessive noise, data dropout, sedation-related confounding, medication-related confounding, insufficient clinical context, insufficient cohort matching, or other reliability conditions. When the signal-quality gate 114 determines that the data fails a reliability threshold, the bedside brain-injury monitoring apparatus 100 can suppress, delay, qualify, or mark a treatment-response classification as indeterminate. This can provide a technical safeguard that prior approaches may lack when they display trend information without assessing whether the underlying signals are reliable enough to support a clinical interpretation.

The brain-state feature table 112 can store features and parameters derived from the EEG waveform data and, in some implementations, from additional patient data. The features can include spectral power, delta power, theta power, alpha power, beta power, gamma power, burst-suppression burden, seizure burden, coherence, connectivity, entropy, rhythmicity, interhemispheric asymmetry, amplitude variability, stimulation reactivity, ICP-derived values, ECG-derived values, or other analytic measures. The brain-state feature table 112 can store such measures in association with time windows, event anchors, patient context, signal-quality flags, medication state, sedation state, and cohort-matching data. Storing the extracted feature representation can allow subsequent comparisons to be performed without repeatedly reprocessing the underlying raw EEG waveform samples.

The treatment-response engine 122 can compare a patient-specific brain-state change to expected recovery information generated from the matched-cohort database 118. For example, the treatment-response engine 122 can identify a pre-event brain-state feature vector associated with a pre-event window 140, identify a post-event brain-state feature vector associated with a post-event window 142, and determine a treatment-response vector representing a change across a treatment event 144. The treatment-response engine 122 can then compare the treatment-response vector to a matched-cohort expected range. Based on the comparison, the treatment-response engine 122 can determine whether the patient is improving, deteriorating, plateauing, deviating from expected recovery, or producing an indeterminate result due to insufficient reliability.

The display 110 can present a recovery trajectory or treatment-response graph. In the illustrated embodiment, the display 110 shows a patient response curve, an expected range based on a matched cohort, and a deviation indicator. The graph can be indexed to days relative to the treatment event, with the treatment event shown as an anchor point. The display 110 can also present a status field, a recovery index, a trend over a defined time period, and a last-updated time. By presenting the patient response relative to a matched cohort and relative to a treatment event, the display 110 can communicate whether the patient's brain-state change is better than expected, expected, or worse than expected. This can differ from approaches that merely show a waveform, a raw EEG trend, a generic health trajectory, or a non-anchored display of clinical values.

The care-team output 130 can include a dashboard, alerts, treatment guidance, reports, notifications, and clinician tools. The care-team output 130 can be generated at the bedside monitoring apparatus 100, at a clinician workstation, on a mobile device, within an electronic health record, or through another clinical communication system. In an exemplary embodiment, the care-team output 130 can identify the treatment event 144, the patient's treatment-response state, confidence level, signal-quality status, contributing signals, deviation metrics, and recommended monitoring actions. The care-team output 130 can be configured to assist the care team in recognizing whether a recent treatment appears to be associated with improvement, deterioration, plateau, deviation, or an indeterminate result.

The lower portion of FIG. 1 illustrates a time-synchronization and event-anchoring concept. The pre-event window 140 can extend before the treatment event 144, for example from about 72 hours before the treatment event 144 to the treatment event 144, although other windows can be used. The post-event window 142 can extend after the treatment event 144, for example from the treatment event 144 to about 72 hours after the treatment event 144, although shorter or longer windows can be used depending on the treatment type, injury type, monitoring frequency, and clinical context. The treatment event 144 can be assigned an anchor time, such as time zero, so that EEG waveform data and other patient data can be evaluated relative to the event.

In an exemplary embodiment, the treatment event 144 can include administration of an anti-seizure medication, administration of a sedative, hyperosmolar therapy, blood-pressure intervention, ventilation adjustment, intracranial-pressure intervention, thrombolytic therapy, thrombectomy, surgical intervention, rehabilitation intervention, neurologic stimulation, or another clinical event. The treatment event 144 can also include a non-treatment event that may affect interpretation, such as seizure onset, deterioration in vital signs, change in sedation state, alarm event, imaging event, or clinical examination event. By anchoring the pre-event window 140 and the post-event window 142 to the treatment event 144, the system 10 can evaluate treatment response in a technically structured manner.

The time synchronization and event anchoring shown in FIG. 1 can also support adaptive monitoring. For example, when the treatment-response engine 122 detects that the patient response is outside the expected range or that the patient is deteriorating, the electronic processor 104 can adjust monitoring parameters, such as sampling rate, feature-extraction interval, alert threshold, waveform-buffer retention period, data-transmission frequency, or cohort-comparison interval. When the patient remains within an expected range for a stability period, the electronic processor 104 can reduce monitoring intensity or reduce data transmission. These operations can improve the operation of the monitoring apparatus 100 itself and can further distinguish the system 10 from approaches that merely calculate or display patient data without controlling monitoring-system operation.

FIG. 1 also illustrates how the system 10 can provide a specific technical improvement in brain-injury monitoring. The system 10 can transform continuous EEG waveform data into feature-based representations, store the feature-based representations in the brain-state feature table 112, gate classifications using the signal-quality gate 114, compare event-anchored patient responses with matched-cohort data from the matched-cohort database 118, and generate a care-team output 130 through the output interface 124. This architecture can reduce the burden of direct raw-waveform interpretation, reduce repeated raw-waveform reprocessing, provide a reliability-aware classification, and present event-specific treatment-response information in a way that can be used during a treatment period following a brain injury.

Referring to FIG. 2, there is illustrated one example of a brain-injury monitoring system in which a bedside monitoring apparatus 100 can receive patient sensor data from one or more sensors 116 and can communicate with one or more matched-cohort/database servers 118 through a secure network. FIG. 2 can focus on the bedside implementation of the system and the flow of information between the patient, the bedside monitoring apparatus 100, the database servers 118, and care-team-facing outputs. In contrast to approaches that merely collect physiologic values or present raw signal trends, the illustrated arrangement can provide a bedside interface that converts patient-specific sensor data into an interpretable brain-state and recovery-status output relative to normative or matched-cohort recovery outcomes.

In an exemplary embodiment, the sensors 116 can include EEG sensors coupled to the patient. The sensors 116 can provide EEG signals to the bedside monitoring apparatus 100. The EEG signals can be raw waveform data, filtered waveform data, segmented waveform data, or other EEG-derived data. The sensors 116 can be configured for continuous acquisition, intermittent acquisition, event-triggered acquisition, or a combination thereof. Although FIG. 2 illustrates sensors 116 positioned on the patient's head, the sensors 116 can additionally or alternatively include intracranial pressure sensors, ECG sensors, arterial-line sensors, oxygenation sensors, respiratory sensors, temperature sensors, movement sensors, or other patient-monitoring sensors.

The sensor inputs shown in FIG. 2 can include EEG, ICP, NIRS, vital signs, and other physiologic information. The EEG input can provide brain electrical activity information. The ICP input can provide intracranial pressure information, such as pressure values or pressure trends. The NIRS input can provide brain oxygenation or perfusion-related information. The vital-sign input can provide heart rate, blood pressure, respiratory rate, oxygen saturation, temperature, or related physiologic data. These inputs can be received by the bedside monitoring apparatus 100 and can be processed individually or in combination to generate a patient-specific brain-state assessment.

The bedside monitoring apparatus 100 can include a display 110 configured to show a recovery trajectory or treatment-response trajectory. As shown, the display 110 can present a patient response, an expected range based on a matched cohort, and a deviation indicator. The display 110 can also identify a treatment event and can show a time axis measured in days relative to the treatment event. The trajectory can indicate whether the patient response is better, expected, or worse relative to the expected range. By anchoring the displayed trajectory to a treatment event, the bedside monitoring apparatus 100 can help distinguish treatment response from a generalized or unanchored physiologic trend.

In the illustrated example, the display 110 can include a status region, a recovery index region, and a trend region. The status region can indicate that a patient is below expected, within expected range, above expected, or indeterminate. The recovery index region can provide a numerical or symbolic value representing a patient's recovery state relative to the expected range. The trend region can indicate whether the patient is improving, deteriorating, plateauing, deviating, or otherwise changing over a defined time period, such as a 24-hour period. The display 110 can also include selectable interface regions, such as alerts, trends, patient, and settings, which can permit a clinician to navigate between alerts, historical trends, patient information, and configuration settings.

The matched-cohort/database servers 118 can store or access recovery models, cohorts, outcome anchors, and model updates. In an exemplary embodiment, the database servers 118 can include one or more local servers, hospital servers, cloud servers, research-network servers, or distributed databases. The recovery models can include expected recovery ranges, treatment-response envelopes, outcome distributions, injury-specific recovery models, or other models derived from prior patients. The cohorts can include prior brain-injury patients selected or filtered based on similarity to the current patient. The outcome anchors can include discharge outcome, functional recovery outcome, neurologic outcome, mortality, morbidity, return-to-activity status, rehabilitation outcome, or other clinically meaningful endpoints. The model updates can allow the database servers 118 to update cohort models as additional patient data, outcomes, or treatment-response data becomes available.

The secure network can provide communication between the bedside monitoring apparatus 100 and the matched-cohort/database servers 118. The secure network can be a hospital network, private network, encrypted network connection, cloud communication link, virtual private network, wireless network, wired network, or other communication pathway. In an exemplary embodiment, the secure network can permit the bedside monitoring apparatus 100 to transmit feature-based patient data, event metadata, or compressed brain-state signatures to the database servers 118. The secure network can also permit the database servers 118 to return matched-cohort comparisons, expected recovery ranges, outcome probabilities, and model or cohort updates to the bedside monitoring apparatus 100.

The data/insights from servers shown in FIG. 2 can include matched-cohort comparisons, expected recovery range data, outcome probabilities, and model/cohort updates. The matched-cohort comparisons can identify how the patient's current or recent brain-state trajectory compares to prior patients having similar injury type, injury severity, treatment-event type, timing from injury onset, physiologic state, imaging pattern, medication state, or other patient-matching parameters. The expected recovery range can define a corridor, envelope, percentile band, or other comparative range against which the patient's response can be evaluated. The outcome probabilities can provide probability values associated with different recovery states or outcomes. The model/cohort updates can refresh or adapt the comparative information used by the bedside monitoring apparatus 100.

In an exemplary embodiment, the bedside monitoring apparatus 100 can transmit less than all raw sensor data to the database servers 118. For example, the bedside monitoring apparatus 100 can transform raw EEG waveform data into features, feature vectors, a brain-state signature, a compressed brain-state signature, or another reduced representation before communication with the database servers 118. This can reduce network burden, reduce repeated remote processing of raw waveform data, and improve privacy or security by limiting transmission of raw patient signals. The database servers 118 can then use the transmitted representation to select or update a matched cohort and return cohort-response data to the bedside monitoring apparatus 100.

The display 110 can indicate current patient brain state or recovery status relative to normative recovery outcomes for guided care. This output can be based on local sensor processing, information returned from the database servers 118, or both. In an exemplary embodiment, the bedside monitoring apparatus 100 can combine EEG-derived features, other sensor inputs, clinical-event timing, and matched-cohort data to determine whether the patient's recovery status is within an expected range, outside an expected range, improving, deteriorating, plateauing, or indeterminate. The displayed status can be generated with a confidence value, signal-quality assessment, or reliability flag so that the care team can understand whether the displayed recovery status is supported by sufficient data quality.

FIG. 2 also illustrates a technical advantage of locating at least part of the processing and display functionality at the bedside. The bedside monitoring apparatus 100 can continue to acquire sensor data, update local trends, display patient response, and provide alerts even if network communication is delayed or unavailable. In some embodiments, cached cohort-response data, stored recovery models, or locally stored comparison information can permit the bedside monitoring apparatus 100 to continue generating a treatment-response output during intermittent network connectivity. When communication with the database servers 118 is restored, the bedside monitoring apparatus 100 can receive updated cohort data, expected recovery ranges, outcome probabilities, or model updates.

The arrangement shown in FIG. 2 can also support adaptive monitoring. For example, the bedside monitoring apparatus 100 can increase monitoring intensity, feature-extraction frequency, waveform-buffer retention, alert sensitivity, or data-transmission frequency when the patient response falls outside the expected range, when a deviation indicator appears, or when the patient is classified as deteriorating. Conversely, the bedside monitoring apparatus 100 can reduce monitoring intensity, reduce data-transmission frequency, or adjust processing frequency when the patient remains stable within an expected recovery range for a defined period. This allows the apparatus 100 to alter its own operation based on the monitored brain-state and treatment-response information.

In use, the sensors 116 can acquire patient signals, the bedside monitoring apparatus 100 can process the sensor inputs and display a recovery trajectory on display 110, the secure network can facilitate communication with the matched-cohort/database servers 118, and the database servers 118 can return comparative data or model updates. The resulting bedside display can provide a care team with a structured, event-aware view of patient recovery rather than requiring the care team to independently interpret raw EEG waveforms, isolated vital signs, or generic population trends. This architecture can provide a technical framework for continuous brain-injury monitoring that integrates bedside signal processing, secure cohort comparison, and clinical decision-support output during a treatment period.

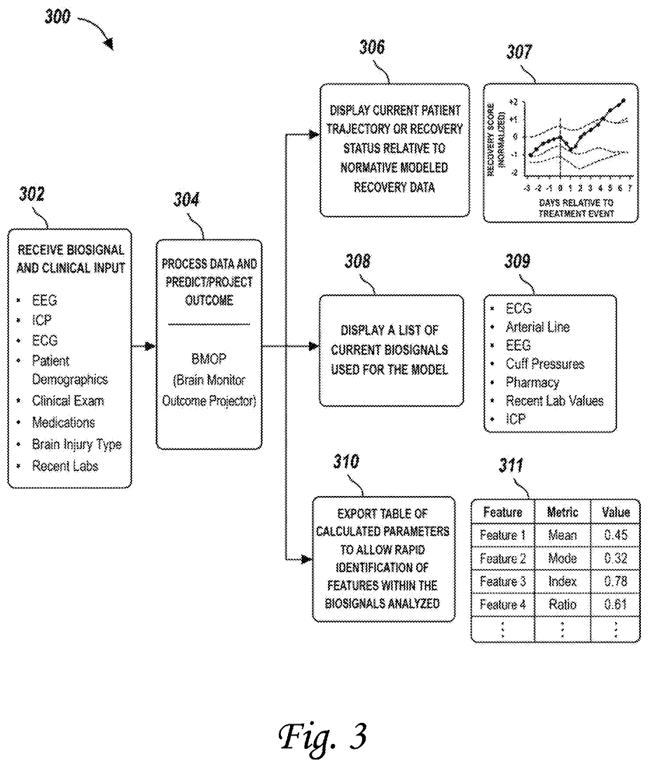

Referring to FIG. 3, there is illustrated one example of a recovery trajectory method 300 for receiving patient-specific biosignal and clinical input 302, processing the input using a processing module 304, and generating one or more outputs that can assist in monitoring a brain-injury patient. FIG. 3 can provide a workflow-level view of how multiple patient data sources can be converted into a recovery trajectory, a list of current biosignals, and a table of calculated parameters. In an exemplary embodiment, the recovery trajectory method 300 can be implemented by the bedside monitoring apparatus 100, the electronic processor 104, the memory 106, the communication interface 108, the display 110, the brain-state feature table 112, the signal-quality gate 114, the treatment-response engine 122, and/or the output interface 124 described with respect to other figures.

The biosignal and clinical input 302 can include data received from one or more patient-monitoring sources and one or more clinical data sources. In the illustrated example, the biosignal and clinical input 302 can include EEG, ICP, ECG, patient demographics, arterial pressures, clinical exam information, pharmacy information, brain injury type, and recent labs. The EEG input can include continuous EEG waveform data or EEG-derived data acquired from one or more EEG sensors. The ICP input can include intracranial pressure measurements or trends. The ECG input can include cardiac rhythm or cardiac electrical activity. The patient demographics can include age, sex, weight, comorbidities, or other patient-specific context. The arterial pressures can include invasive or non-invasive pressure measurements. The clinical exam information can include neurological examination findings, Glasgow Coma Scale score, National Institutes of Health Stroke Scale score, pupillary findings, motor response, speech response, or other bedside clinical observations. The pharmacy information can include medications, dosing, timing, medication class, sedative state, anti-seizure medication state, or other pharmacologic context. The brain injury type can identify traumatic brain injury, ischemic stroke, hemorrhagic stroke, hypoxic-ischemic injury, subarachnoid hemorrhage, contusion, diffuse axonal injury, or another neurologic injury. The recent labs can include laboratory values relevant to physiologic state, inflammation, metabolic status, coagulation, oxygenation, or treatment response.

In an exemplary embodiment, the biosignal and clinical input 302 can be received continuously, intermittently, on demand, in response to an event, or according to a monitoring protocol. The biosignal and clinical input 302 can be received from bedside sensors, an electronic health record, medication-administration records, imaging systems, laboratory systems, ventilators, infusion pumps, intracranial pressure monitors, clinician-entered data, or other systems. The use of multiple input types can allow the recovery trajectory method 300 to evaluate the patient's brain state in clinical context rather than relying on a single isolated signal or a generic health score.

The processing module 304 can process the biosignal and clinical input 302 and can predict or project an outcome. In the illustrated embodiment, the processing module 304 can be a BMOP, or Brain Monitor Outcome Projector. The processing module 304 can analyze waveform data, clinical data, treatment-event data, and patient context to generate one or more brain-injury recovery outputs. In some implementations, the processing module 304 can transform raw waveform data into analytic measures, generate feature vectors, normalize features relative to baseline, compare patient features to normative or matched-cohort data, and generate a recovery trajectory or recovery-state assessment. The processing module 304 can also generate treatment-response information when the input data is anchored to a treatment event, clinical event, or other time reference.

The processing module 304 can be configured to reduce the need for direct manual interpretation of raw EEG waveform data. For example, instead of requiring a clinician to review long segments of EEG waveforms, the processing module 304 can extract analytic measures from waveform data, store the analytic measures in a feature table, and evaluate feature changes over time. This can provide a technical improvement over approaches that display raw signals or isolated vital signs without converting the signals into a structured feature representation for recovery assessment. The processing module 304 can also use signal-quality evaluation, artifact rejection, missing-data handling, or confidence scoring so that subsequent outputs are not treated as reliable when the underlying data is insufficient.

In an exemplary embodiment, the processing module 304 can generate one or more outputs, including a trajectory/status display step 306, a biosignal list display step 308, and a calculated-parameter export step 310. These outputs can be generated separately, simultaneously, sequentially, or in response to user selection. The recovery trajectory method 300 can therefore provide different levels of explanation for a clinician. A first output can show an overall trajectory or recovery status, a second output can identify the current biosignals being used, and a third output can provide calculated parameters that allow the care team to understand which features contributed to the analysis.

The trajectory/status display step 306 can display a current patient trajectory or recovery status relative to normative modeled recovery data. The trajectory/status display step 306 can generate or update a recovery trajectory graph 307. The recovery trajectory graph 307 can show a patient's current recovery state, a trend over time, and/or a position relative to a modeled recovery region. In some embodiments, the normative modeled recovery data can be generated from prior patients, matched cohorts, injury-specific recovery models, treatment-event-specific recovery models, or combinations thereof. The trajectory/status display step 306 can allow a care team to determine whether the patient is progressing within an expected range, improving more rapidly than expected, deteriorating, plateauing, or deviating from an expected recovery path.

The recovery trajectory graph 307 can be generated based on analytic measures derived from EEG and other patient data. For example, the recovery trajectory graph 307 can include a patient response line, a matched-cohort range, one or more expected recovery boundaries, a deviation indicator, or another graphical representation of recovery status. Although FIG. 3 shows the recovery trajectory graph 307 as a compact trajectory display, the graph can alternatively be displayed as a larger dashboard, timeline, confidence plot, percentile band, response envelope, or trend visualization. The recovery trajectory graph 307 can be updated as new biosignal data, clinical information, or treatment-event information is received.

The biosignal list display step 308 can display a list of current biosignals used for the model. The current biosignal list 309 can identify which signals or data sources are currently being used by the processing module 304. In the illustrated example, the current biosignal list 309 can include ECG, arterial line, EEG, cuff pressures, pharmacy data, recent lab values, and ICP. The current biosignal list 309 can provide transparency into the model inputs and can help a care team determine whether a generated recovery trajectory or treatment-response output was based on adequate and relevant patient data. In some embodiments, the current biosignal list 309 can further display, or provide access to, one or more input-importance indicators associated with the listed biosignals or derived features. The input-importance indicators can include, for example, feature-weight values, model attention weights, feature-attribution values, SHAP values, permutation-importance values, confidence scores, data-quality scores, missing-data flags, signal-noise indicators, artifact indicators, or recency indicators.

When imaging-derived features are used by the processing module 304, the current biosignal list 309 can identify the imaging source and one or more imaging-derived features supplied to the model, such as lesion volume, midline shift, edema volume, hemorrhage volume, ventricular size, perfusion parameters, diffusion parameters, or segmented anatomical-region measurements. The system can further present one or more imaging-quality or segmentation-quality indicators, including, for example, a DICE similarity score, intersection-over-union score, segmentation-confidence score, registration-quality score, image-acquisition timestamp, modality identifier, or image-artifact indicator. These indicators can allow the care team to determine whether an imaging-derived feature materially influenced the recovery trajectory or treatment-response output and whether the imaging-derived feature was generated from sufficiently reliable image data.

In some embodiments, the processing module 304 can use the input-importance indicators, data-quality scores, or imaging-quality indicators to generate an output confidence level, an adequacy warning, or a recommendation to acquire additional patient data before relying on the generated recovery trajectory or treatment-response output. For example, the system can indicate that a treatment-response output was generated without recent ICP data, with low-quality EEG data, or with an imaging-derived lesion-volume feature having a segmentation-quality score below a threshold.

In an exemplary embodiment, the current biosignal list 309 can also include indicators showing whether a signal is active, missing, unreliable, excluded, down-weighted, or flagged for artifact. For example, the EEG input can be marked unreliable when electrode impedance exceeds a threshold, the arterial-line input can be marked missing when the line is disconnected, or the pharmacy input can be marked active when a medication event has been received. The current biosignal list 309 can thereby support quality-gated operation and can help prevent overreliance on a recovery trajectory when important input data is absent or unreliable.

The calculated-parameter export step 310 can export a table of calculated parameters to allow rapid identification of features within the analyzed biosignals. The calculated-parameter table 311 can include features, metrics, values, indices, ratios, summary statistics, or other extracted parameters. In the illustrated example, the calculated-parameter table 311 can include feature names, metrics, and values. The calculated parameters can include mean values, modes, ratios, indices, kurtosis values, high/low/normal classifications, spectral-power values, entropy values, coherence values, asymmetry values, seizure-burden values, burst-suppression values, ICP trend values, ECG-derived values, or other analytic measures.

The calculated-parameter table 311 can provide a technical bridge between raw physiologic data and the recovery trajectory graph 307. Instead of treating the recovery trajectory graph 307 as a black-box output, the calculated-parameter table 311 can expose the features used by the processing module 304. This can be useful for clinical review, model auditing, regulatory documentation, error detection, and treatment-response interpretation. For example, if the recovery trajectory graph 307 indicates deterioration, the calculated-parameter table 311 can allow a clinician to see whether the deterioration was driven by EEG slowing, increased asymmetry, increased seizure burden, elevated ICP, worsening arterial pressure, or another contributing feature.

The calculated-parameter export step 310 can also support efficient system operation. In some implementations, the processing module 304 can convert raw waveform data into analytic measures and store those measures in a structured table, such as the brain-state feature table 112. Subsequent recovery trajectory calculations, treatment-response classifications, matched-cohort comparisons, and dashboard displays can then use the stored calculated parameters rather than repeatedly reprocessing the raw waveform data. This can reduce processing burden, reduce latency, reduce memory or network usage, and support continuous bedside operation.

In an exemplary embodiment, the recovery trajectory method 300 can include applying a signal-quality gate before displaying the recovery trajectory graph 307, displaying the current biosignal list 309, or exporting the calculated-parameter table 311. The signal-quality gate can determine whether the EEG data, ICP data, ECG data, arterial pressure data, clinical-event data, or other input data satisfies one or more reliability thresholds. If a threshold is not satisfied, the method 300 can mark the output as indeterminate, display a confidence warning, omit one or more unreliable features, or request additional data. This can be particularly useful in neurocritical care, where raw signals may be affected by motion artifact, poor electrode contact, muscle artifact, sedation, procedures, or missing channels.

The workflow of FIG. 3 can support event-anchored recovery assessment even though FIG. 3 is shown generally as a recovery trajectory method. In some embodiments, the processing module 304 can associate the biosignal and clinical input 302 with a time-stamped treatment event. The processing module 304 can then generate analytic measures before and after the event, compare the resulting measures, and update the recovery trajectory graph 307 based on whether the patient response is consistent with an expected treatment-response envelope. In this manner, the recovery trajectory method 300 can be used not only to monitor general recovery but also to determine whether a treatment appears to be associated with improvement, deterioration, plateau, deviation, or an indeterminate response.

The illustrated method 300 can also differentiate the present system from prior approaches that may provide generalized health trajectory information, manual EEG interpretation, or isolated comparative case displays. The processing module 304 can use brain-injury-specific inputs, such as EEG, ICP, brain injury type, clinical examination findings, and treatment timing, to generate a recovery trajectory or treatment-response output tailored to a brain-injury patient. The current biosignal list 309 and calculated-parameter table 311 can provide transparency and technical grounding for the output. The recovery trajectory graph 307 can present the processed result in a form that is understandable at the bedside while still being derived from continuous waveform and clinical data.

In use, the recovery trajectory method 300 can receive biosignal and clinical input 302, process the input at the processing module 304, display a patient trajectory or recovery status at the trajectory/status display step 306 and recovery trajectory graph 307, display the current biosignal list 309 through the biosignal list display step 308, and export the calculated-parameter table 311 through the calculated-parameter export step 310. The method 300 can thereby provide a layered output architecture in which a care team can see an overall patient recovery state, confirm which signals were used, and review the analytic parameters supporting the system output.

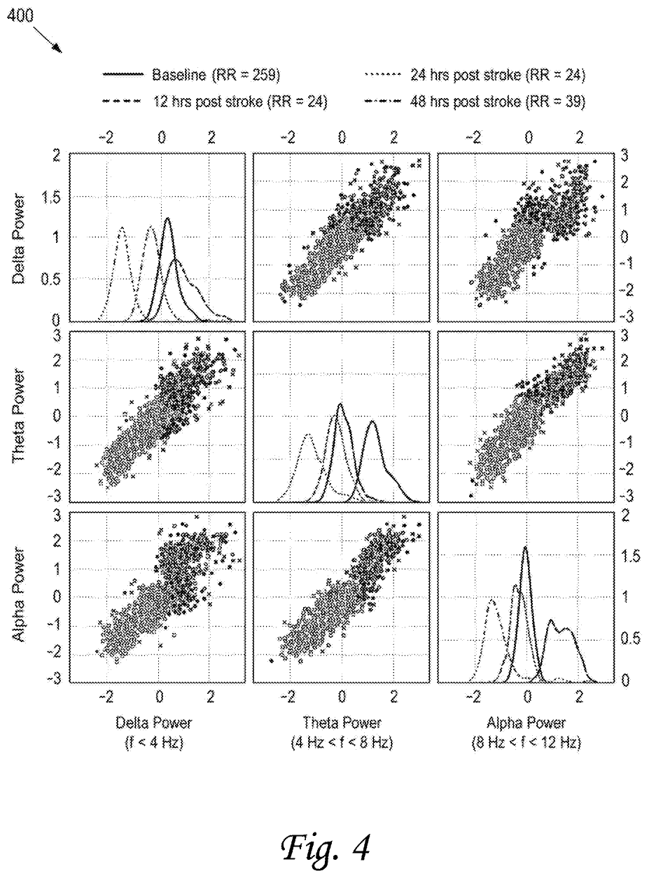

Referring to FIG. 4, there is illustrated one example of an EEG power analysis table 400 including a plurality of frequency-based EEG power features extracted from monitored EEG data. FIG. 4 can provide an example of how raw or continuous EEG waveform data can be transformed into analytic measures that are more suitable for computational comparison, classification, cohort matching, and recovery-trajectory analysis. In an exemplary embodiment, the EEG power analysis table 400 can be generated by processing EEG waveform data received from one or more EEG sensors 116 and can include frequency-domain features that may be stored in the brain-state feature table 112, used by the treatment-response engine 122, or provided to one or more classifiers such as the EEG data classifier 506C.

The EEG power analysis table 400 can include a multi-panel matrix of plotted relationships between EEG power features. In the illustrated example, the features include delta power, theta power, and alpha power. Delta power can correspond to EEG power in a frequency band below about 4 Hz, theta power can correspond to EEG power in a frequency band between about 4 Hz and about 8 Hz, and alpha power can correspond to EEG power in a frequency band between about 8 Hz and about 12 Hz. Other bands, sub-bands, ratios, or transformed frequency-domain features can also be used, including beta power, gamma power, delta-to-alpha ratio, alpha-to-theta ratio, spectral edge frequency, band-power asymmetry, or other spectral measures.

In an exemplary embodiment, the EEG power analysis table 400 can compare EEG-derived power features at different time points or clinical states. As shown, the table 400 can include data associated with a baseline state, a 12-hour post-stroke state, a 24-hour post-stroke state, and a 48-hour post-stroke state. These time points can be exemplary and can be replaced or supplemented with time points relative to a brain injury event, a treatment event, a seizure event, a medication event, a ventilation change, an intracranial-pressure intervention, or another clinical event. In the context of the present system, the same type of frequency-based analysis can be applied to a pre-event window 140 and a post-event window 142 relative to a treatment event 144.

The diagonal panels of the EEG power analysis table 400 can show distributions of individual EEG power features. For example, a first diagonal panel can show a distribution of delta power, a second diagonal panel can show a distribution of theta power, and a third diagonal panel can show a distribution of alpha power. Each distribution can reflect how a particular frequency-domain feature changes across different monitoring states or time periods. A shift in one or more of these distributions can indicate a change in brain state, such as slowing, recovery, deterioration, seizure-related change, ischemic evolution, sedation effect, or other neurologic or physiologic change.

The off-diagonal panels of the EEG power analysis table 400 can show pairwise relationships between EEG power features. For example, one panel can show delta power relative to theta power, another panel can show delta power relative to alpha power, and another panel can show theta power relative to alpha power. These pairwise plots can capture relationships that may not be apparent from a single feature alone. In some implementations, clustering, dispersion, slope, covariance, correlation, or separation between plotted points can provide information about the current brain state or the direction of recovery. These relationships can be used as analytic measures, classifier inputs, or components of a brain-state signature.

The plotted data in FIG. 4 can demonstrate that changes in EEG waveform content can be represented as measurable shifts in feature space. In an exemplary embodiment, the electronic processor 104 can transform EEG waveform samples into frequency-domain features and can store the resulting features as entries in the brain-state feature table 112. The stored features can include power values, normalized power values, ratios, distribution statistics, covariance values, cluster locations, or other quantitative descriptors derived from the plotted relationships. Once the EEG waveform data has been transformed into such feature-based representations, later processing can use the stored analytic measures without repeatedly reprocessing the underlying waveform samples.

The EEG power analysis table 400 can also support generation of a temporally anchored brain-state signature. For example, the electronic processor 104 can compute delta power, theta power, alpha power, and related feature relationships during a baseline window, during the pre-event window 140, during the post-event window 142, or during successive time windows after the treatment event 144. The resulting features can be aggregated, normalized, and stored as a feature vector or brain-state signature associated with a particular time window and event anchor. Changes between the pre-event feature values and post-event feature values can be used to determine a treatment-response vector.

In an exemplary embodiment, the frequency-based EEG features shown in FIG. 4 can be used with clinical context data to distinguish different recovery patterns. For example, an increase or decrease in a particular band-power measure may have different significance depending on injury type, sedation state, medication state, intracranial pressure, seizure burden, imaging findings, or time from injury onset. The system 10 can therefore use the EEG power analysis table 400 as one feature source within a broader brain-injury treatment-response monitoring architecture rather than treating a single EEG metric as a standalone diagnostic output.

The EEG power analysis table 400 can also be used to compare a current patient with prior patients. In some implementations, features derived from the table 400 can be transmitted or otherwise provided to the matched-cohort database 118 or the matched brain-injury cohort selection engine 1100. A matched cohort can be selected based in part on similarity of EEG-derived spectral features, feature trajectories, or post-event shifts in feature space. The selected cohort can then be used to generate an expected response envelope 910 or expected intervention-response envelope for comparison with the current patient's actual response 912.

This type of transformation can provide an important technical distinction from approaches that simply display an EEG waveform or require a human reviewer to interpret raw EEG data. The EEG power analysis table 400 can show how continuous EEG waveform data can be converted into structured, quantifiable, and comparable analytic measures. The structured analytic measures can be stored, queried, compared to matched-cohort data, and used as classifier inputs. This can reduce dependence on manual waveform interpretation and can allow the system 10 to provide a more objective and repeatable recovery or treatment-response output.

In some implementations, the EEG power analysis table 400 can be generated using a windowed frequency analysis. The EEG waveform data can be segmented into epochs or windows, filtered, transformed into the frequency domain, and summarized for each selected frequency band. The analysis can use Fourier transforms, wavelet transforms, multitaper spectral estimation, autoregressive modeling, time-frequency decomposition, or other signal-processing techniques. The resulting measures can be normalized to patient baseline, cohort baseline, contralateral hemisphere values, age-adjusted norms, injury-specific norms, or other reference values.

The table 400 can also support artifact and reliability evaluation. For example, abnormal shifts in spectral power may be caused by electrode artifact, muscle activity, movement, environmental noise, missing channels, or other non-brain sources. In an exemplary embodiment, the signal-quality gate 114 can evaluate whether the EEG features represented in the table 400 are reliable enough to be used in a recovery trajectory or treatment-response classification. If the signal-quality gate 114 determines that the spectral features are unreliable, the system 10 can omit the features, down-weight the features, mark the output as low confidence, or classify the response as indeterminate.

The frequency-based features shown in FIG. 4 can further support machine-learning classification. For example, one or more classifiers can be trained to evaluate feature distributions, feature shifts, feature ratios, or feature trajectories generated from the EEG power analysis table 400. The EEG data classifier 506C can receive the features and output a probability associated with one or more injury-severity classes, recovery states, or treatment-response states. In some embodiments, the EEG data classifier 506C can be used together with text data classifier 506A and imaging data classifier 506B, and the outputs can be combined by decision fusion 508 to generate a final decision output 510.

The EEG power analysis table 400 can be particularly useful for monitoring brain injury because brain-state changes may occur continuously and may not be fully captured by intermittent examinations or episodic imaging. By repeatedly generating or updating feature measures like those shown in FIG. 4, the system 10 can track how a patient's brain function evolves over minutes, hours, or days. The resulting feature trajectories can help detect improvement, deterioration, plateau, or deviation from an expected response, especially when evaluated relative to treatment-event timing and matched-cohort recovery data.

Although FIG. 4 illustrates delta power, theta power, and alpha power in a post-stroke example, the same framework can be applied to traumatic brain injury, hemorrhagic stroke, hypoxic-ischemic injury, subarachnoid hemorrhage, diffuse axonal injury, contusion, post-operative brain injury, seizure-related injury, or other neurologic conditions. The frequency bands, time windows, plotted features, and plotted relationships can be selected based on the patient, the injury type, the treatment event, the available sensors, or a monitoring protocol. The figure therefore provides an example of a technical feature-extraction approach that can support the broader treatment-response monitoring system disclosed herein.

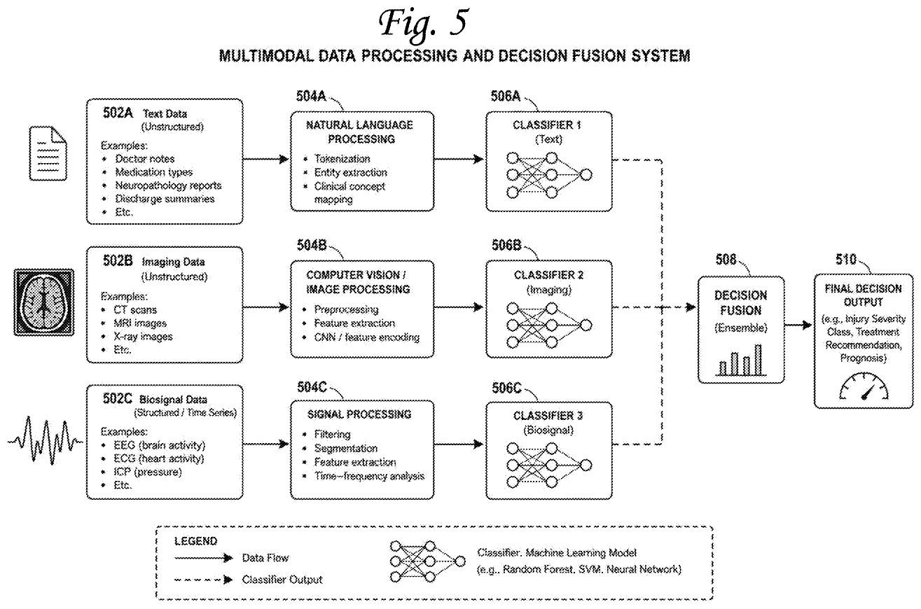

Referring to FIG. 5, there is illustrated one example of a multimodal data processing system 500 for determining a brain-injury recovery assessment using a plurality of patient-data streams. In the illustrated embodiment, the multimodal data processing system 500 can receive text data 502A, imaging data 502B, and biosignal data 502C. The text data 502A can be processed by natural language processing 504A and evaluated by a text data classifier 506A. The imaging data 502B can be processed by computer vision/image processing 504B and evaluated by an imaging data classifier 506B. The biosignal data 502C can be processed by signal processing 504C and evaluated by an EEG/biosignal data classifier 506C. Outputs from the classifiers 506A, 506B, 506C can be combined by decision fusion 508 to produce a final decision output 510.

In an exemplary embodiment, the multimodal data processing system 500 can be implemented by the electronic processor 104, the memory 106, the communication interface 108, the brain-state feature table 112, the treatment-response engine 122, and/or the output interface 124. The multimodal data processing system 500 can use different processing pipelines for different types of patient data so that heterogeneous information associated with a brain-injury patient can be converted into classifier-compatible representations. By separating text, imaging, and biosignal processing before decision fusion 508, the system can reduce the likelihood that dissimilar data types are forced into a single generic model without preserving clinically and technically meaningful differences between the data streams.

The text data 502A can include doctor notes, types of medication, neuropathology reports, clinical exam notes, nursing notes, procedure notes, medication-administration records, radiology reports, discharge summaries, rehabilitation notes, or other text-format clinical information. The natural language processing 504A can extract structured information from the text data 502A, such as injury type, symptom onset time, mechanism of injury, neurologic examination findings, sedation state, medication state, procedure timing, treatment-event type, treatment-event time, complications, and outcome information. In some implementations, the natural language processing 504A can identify time-stamped treatment events that can be used to define the pre-event window 140 and the post-event window 142 relative to the treatment event 144.

The text data classifier 506A can evaluate the processed text data and can generate a text-based classification output. The text-based classification output can include one or more probabilities, scores, labels, or confidence values associated with injury severity, recovery state, treatment-response state, cohort-matching information, or reliability of a clinical event. For example, the text data classifier 506A can identify that a patient received an anti-seizure medication at a particular time, that the patient's examination improved after the medication, or that the patient was sedated during a relevant EEG window. The text data classifier 506A can thereby provide context that may alter interpretation of the EEG or other biosignal data.

The imaging data 502B can include fMRI, TCD, CT, MRI, angiography, perfusion imaging, ultrasound, X-ray, or other imaging data. The imaging data 502B can provide information regarding brain structure, blood flow, perfusion, hemorrhage, ischemia, edema, midline shift, vessel occlusion, infarct core, penumbra, contusion burden, diffuse axonal injury, or another imaging feature associated with brain injury. The computer vision/image processing 504B can extract imaging features from the imaging data 502B, including lesion location, lesion volume, vascular territory, perfusion deficit, hemorrhage volume, edema burden, ventricular size, or other image-derived features.

The imaging data classifier 506B can evaluate the processed imaging data and can generate an imaging-based classification output. The imaging-based classification output can include a severity probability, recovery-state probability, outcome probability, cohort-selection score, treatment-response modifier, or imaging-context label. In an exemplary embodiment, the imaging data classifier 506B can help determine whether a current patient should be compared to a cohort of prior patients with similar imaging findings. For example, a patient having a large-vessel occlusion and thrombectomy event may be compared to a different matched brain-injury cohort than a patient having diffuse traumatic axonal injury or hypoxic-ischemic injury.

The biosignal data 502C can include EEG brain-activity data, ECG heart-activity data, ICP data, arterial pressure data, pulse oximetry data, respiratory data, temperature data, NIRS data, movement data, or other time-series physiologic data. The signal processing 504C can filter, segment, normalize, transform, or otherwise process the biosignal data 502C. In some implementations, the signal processing 504C can transform raw EEG waveform data into analytic measures, such as spectral power, interhemispheric asymmetry, burst-suppression burden, seizure burden, coherence, entropy, rhythmicity, amplitude variability, stimulation reactivity, or other EEG-derived features.

The EEG/biosignal data classifier 506C can evaluate processed biosignal features and can generate a biosignal-based classification output. The biosignal-based classification output can identify a probability or score associated with injury severity, recovery state, treatment-response state, deterioration, plateau, deviation from expected recovery, or indeterminate status. In some embodiments, the EEG/biosignal data classifier 506C can compare a pre-event brain-state feature vector to a post-event brain-state feature vector to determine a treatment-response vector. The EEG/biosignal data classifier 506C can also evaluate trends in biosignal features across minutes, hours, or days to identify evolving changes in brain state.

In an exemplary embodiment, each classifier 506A, 506B, 506C can be trained or configured for a particular data modality. This can provide a technical advantage because text data, imaging data, and biosignal data can have different data structures, temporal characteristics, noise sources, missing-data patterns, and clinical meanings. The text data classifier 506A can address unstructured or semi-structured language. The imaging data classifier 506B can address spatial image features. The EEG/biosignal data classifier 506C can address continuous waveform and time-series features. The decision fusion 508 can then combine the classifier outputs after each modality has been converted into a modality-appropriate representation.

The decision fusion 508 can combine outputs of the classifiers 506A, 506B, 506C to generate the final decision output 510. The decision fusion 508 can use averaging, weighted averaging, voting, Bayesian fusion, ensemble modeling, stacking, rule-based fusion, confidence-weighted fusion, time-windowed fusion, or another fusion technique. In some implementations, the decision fusion 508 can weight a classifier output based on signal quality, data availability, recency, reliability, injury type, treatment-event type, cohort match quality, or clinical context. For example, the output of the EEG/biosignal data classifier 506C can be down-weighted when EEG quality is poor, while the output of the text data classifier 506A can be down-weighted when clinical documentation is incomplete or delayed.

The final decision output 510 can include an injury-severity assessment, recovery assessment, recovery trajectory, treatment-response classification, deterioration alert, deviation flag, predicted outcome, confidence indicator, or other clinical-management output. In the context of the treatment-response monitoring system 10, the final decision output 510 can identify whether a patient is improving, deteriorating, plateauing, deviating from expected recovery, or indeterminate. The final decision output 510 can also include a confidence value and can identify which modalities contributed to the output.

The multimodal structure of FIG. 5 can support a more reliable interpretation of brain-injury recovery than an approach that relies solely on one signal type. For example, EEG slowing after a treatment event may indicate deterioration in some circumstances, but the same EEG change may be explained by sedation if medication information extracted from the text data 502A indicates that a sedative was recently administered. Similarly, imaging data 502B may indicate a lesion pattern that changes the expected recovery envelope used to interpret biosignal data 502C. By processing each modality through a dedicated pipeline and combining the outputs through decision fusion 508, the system can generate a more context-aware and technically grounded output.

In an exemplary embodiment, the multimodal data processing system 500 can operate with missing or unreliable modalities. If the imaging data 502B is unavailable, the decision fusion 508 can combine outputs from the text data classifier 506A and the EEG/biosignal data classifier 506C. If the EEG/biosignal data classifier 506C is marked unreliable by the signal-quality gate 114, the decision fusion 508 can reduce the weight of the biosignal output or classify the final decision output 510 as indeterminate. This can allow the system to operate continuously while still recognizing limitations in the underlying data.

The signal processing 504C and EEG/biosignal data classifier 506C can also support the technical improvement of transforming waveform data into a structured feature representation. Rather than transmitting or repeatedly analyzing raw EEG waveform data for every comparison, the signal processing 504C can generate feature vectors, brain-state signatures, or other compressed representations. These representations can be stored in the brain-state feature table 112 and later used by the decision fusion 508, the treatment-response engine 122, the matched-cohort database 118, or the matched brain-injury cohort selection engine 1100. This can reduce computational burden and support faster generation of bedside treatment-response outputs.

FIG. 5 can also support matched-cohort recovery analysis. Outputs from the text data classifier 506A, imaging data classifier 506B, and EEG/biosignal data classifier 506C can be used to select a matched brain-injury cohort. For example, text-derived injury and event information, imaging-derived lesion features, and EEG-derived brain-state features can be used together to identify prior patients having similar injury type, severity, time from injury onset, treatment-event type, and physiologic state. The selected cohort can then be used to generate expected response information for comparison with the current patient.

The multimodal data processing system 500 can be used during continuous monitoring at the bedside. New text data 502A, imaging data 502B, and biosignal data 502C can be received over time and processed through their respective pipelines. The decision fusion 508 can update the final decision output 510 as new data becomes available. In some embodiments, the final decision output 510 can be updated when a new treatment event is received, when a new post-event time window becomes available, when signal quality changes, when new imaging is obtained, or when new clinical documentation is entered. This can allow the system 10 to provide an evolving recovery or treatment-response assessment during the treatment period.

The architecture shown in FIG. 5 can differ from prior approaches that rely on generic health-trajectory questions, isolated EEG frequency calculations, or visual comparison of examination values. The illustrated system can combine modality-specific processing, brain-injury-specific classification, and decision fusion to generate a practical treatment-response or recovery-management output. This can support the overall technical narrative that the disclosed system does not merely receive and display medical data, but instead transforms multiple types of patient data into modality-specific classifier outputs, applies fusion logic, and generates a structured brain-injury recovery assessment.

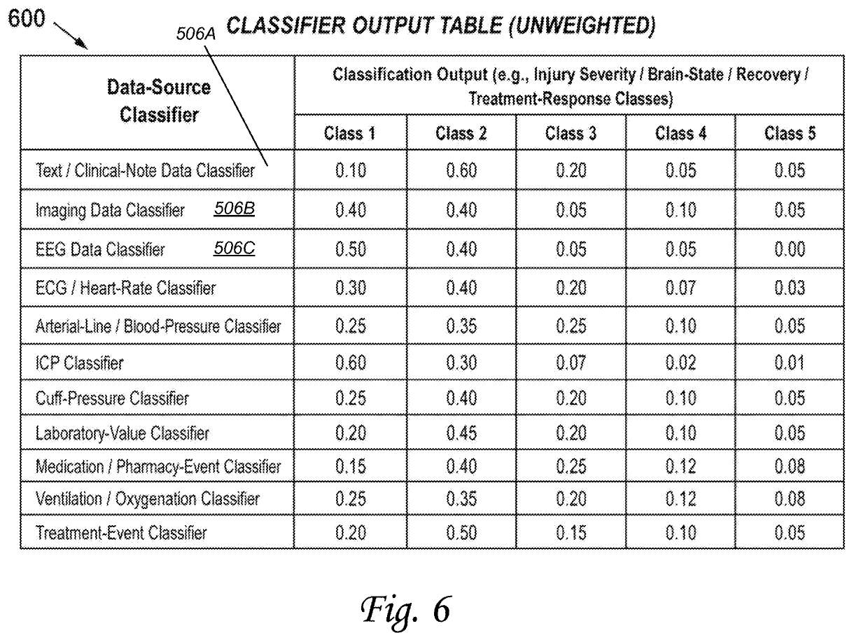

Referring to FIG. 6, there is illustrated one example of a classifier output table 600 generated by the processing module for organizing classification outputs from a plurality of patient-data classifiers. The classifier output table 600 can be generated as an intermediate data structure, a displayed user-interface element, a stored record, a transmitted message, or a model-input transparency report. In the illustrated embodiment, the classifier output table 600 includes a left-side data-source classifier column and a plurality of classification-output columns. The classifier output table 600 can be used to show, for each patient-data source or patient-data modality, how strongly that source supports each of a plurality of classification classes.

The classifier output table 600 includes a table identifier 600. The table identifier 600 can be used in the drawings and specification to refer to the overall unweighted classifier-output structure. In this example, the table 600 is labeled as a “Classifier Output Table (Unweighted),” indicating that the values in the table are classifier outputs before application of weighting factors, reliability adjustments, signal-quality adjustments, patient-specific relevance adjustments, or other weighting operations. Thus, the table 600 can represent an initial set of classifier outputs generated from the available patient data before the outputs are fused or aggregated into a final classification result.

The classifier output table 600 includes a first column labeled “Data-Source Classifier.” The data-source classifier column identifies the particular classifier, data stream, data modality, or derived-data category responsible for generating the corresponding row of class-output values. In the illustrated example, the data-source classifier column includes a text/clinical-note data classifier, an imaging data classifier, an EEG data classifier, an ECG/heart-rate classifier, an arterial-line/blood-pressure classifier, an intracranial-pressure classifier, a cuff-pressure classifier, a laboratory-value classifier, a medication/pharmacy-event classifier, a ventilation/oxygenation classifier, and a treatment-event classifier. These rows demonstrate that the classification system can combine not only conventional text, imaging, and EEG data, but also bedside physiologic data streams, medication events, laboratory data, respiratory data, and treatment-event information.

The classifier output table 600 further includes a classification-output header extending across a plurality of columns. In the illustrated embodiment, the classification-output header identifies the columns as classification outputs, such as injury-severity classes, brain-state classes, recovery classes, treatment-response classes, or other patient-state classes. The use of multiple possible class meanings is intended to indicate that the same table structure can be used in different embodiments. For example, in one implementation, Class 1 through Class 5 can correspond to increasing injury-severity levels. In another implementation, Class 1 through Class 5 can correspond to different brain-state categories. In another implementation, Class 1 through Class 5 can correspond to recovery-trajectory categories, such as faster-than-expected recovery, expected recovery, delayed recovery, worsening trajectory, or indeterminate response. In still another implementation, the classes can represent treatment-response classifications, deviation-risk classifications, or expected-recovery-envelope classifications.

In the illustrated example, the classification-output columns are labeled Class 1, Class 2, Class 3, Class 4, and Class 5. Each class column contains a classifier output value for a corresponding data-source classifier row. The output value can be a probability, confidence value, normalized score, classifier-distribution value, class-membership value, likelihood value, severity score, recovery-state value, treatment-response value, deviation-risk value, or other numerical model output. In some embodiments, the values in each row can sum to one so that each row represents a probability distribution generated by the corresponding classifier. In other embodiments, the values can be unnormalized scores that are later normalized by the processing module.

The first row of the classifier output table 600 can correspond to the text/clinical-note data classifier. In the illustrated example, the text/clinical-note data classifier has output values of 0.10 for Class 1, 0.60 for Class 2, 0.20 for Class 3, 0.05 for Class 4, and 0.05 for Class 5. This row can indicate that, based on text or clinical-note data, the classifier most strongly supports Class 2, with secondary support for Class 3 and lower support for Classes 1, 4, and 5. The text/clinical-note data classifier can process unstructured or structured text, including emergency department notes, admission notes, operative reports, nursing notes, physician assessments, progress notes, radiology text reports, medication notes, discharge summaries, or structured clinical fields. The resulting row can allow the system or care team to see how the text-derived information contributes to the overall classification.

The second row of the classifier output table 600 can correspond to the imaging data classifier. In the illustrated example, the imaging data classifier has output values of 0.40 for Class 1, 0.40 for Class 2, 0.05 for Class 3, 0.10 for Class 4, and 0.05 for Class 5. This row can indicate that imaging-derived data provides approximately equal support for Class 1 and Class 2, lower support for Class 4, and still lower support for Classes 3 and 5. The imaging data classifier can operate on image data or imaging-derived features from computed tomography, magnetic resonance imaging, ultrasound, angiography, perfusion imaging, diffusion imaging, or other imaging modalities. The imaging-derived features can include lesion volume, hemorrhage volume, edema volume, midline shift, ventricular size, perfusion parameters, diffusion parameters, anatomical-region measurements, or segmentation-derived measurements.

The third row of the classifier output table 600 can correspond to the EEG data classifier. In the illustrated example, the EEG data classifier has output values of 0.50 for Class 1, 0.40 for Class 2, 0.05 for Class 3, 0.05 for Class 4, and 0.00 for Class 5. This row can indicate that EEG-derived information most strongly supports Class 1, also supports Class 2, and provides little or no support for the remaining classes. The EEG data classifier can process raw EEG waveforms, spectral power features, seizure-detection outputs, burst-suppression indicators, asymmetry measurements, slowing measurements, artifact-filtered EEG features, or other neurophysiologic features derived from electroencephalography.

The fourth row of the classifier output table 600 can correspond to the ECG/heart-rate classifier. In the illustrated example, the ECG/heart-rate classifier has output values of 0.30 for Class 1, 0.40 for Class 2, 0.20 for Class 3, 0.07 for Class 4, and 0.03 for Class 5. This row can indicate that cardiac-derived information most strongly supports Class 2, with meaningful but lower support for Classes 1 and 3. The ECG/heart-rate classifier can process heart rate, heart-rate variability, rhythm classification, arrhythmia indicators, electrocardiogram waveform features, autonomic-response indicators, or other cardiac features. In certain embodiments, these cardiac features can reflect physiologic stress, autonomic instability, response to medication, deterioration, or improvement following a treatment event.

The fifth row of the classifier output table 600 can correspond to the arterial-line/blood-pressure classifier. In the illustrated example, the arterial-line/blood-pressure classifier has output values of 0.25 for Class 1, 0.35 for Class 2, 0.25 for Class 3, 0.10 for Class 4, and 0.05 for Class 5. This row can indicate that invasive hemodynamic measurements support Class 2 most strongly, while also providing support for Classes 1 and 3. The arterial-line/blood-pressure classifier can process systolic pressure, diastolic pressure, mean arterial pressure, pulse pressure, arterial waveform morphology, pulse-pressure variation, pressure trends, hypotensive events, hypertensive events, or other hemodynamic features. In some embodiments, the arterial-line/blood-pressure row can be used together with intracranial-pressure data to derive or classify cerebral perfusion pressure or pressure-reactivity status.

The sixth row of the classifier output table 600 can correspond to the intracranial-pressure classifier. In the illustrated example, the intracranial-pressure classifier has output values of 0.60 for Class 1, 0.30 for Class 2, 0.07 for Class 3, 0.02 for Class 4, and 0.01 for Class 5. This row can indicate that intracranial-pressure data strongly supports Class 1 and provides lesser support for Class 2 and the remaining classes. The intracranial-pressure classifier can process measured ICP values, ICP trends, ICP waveform morphology, plateau-wave events, pressure-reactivity values, variability metrics, threshold-exceedance duration, response to osmotherapy, response to drainage, or other ICP-derived features. This row can be especially useful in embodiments where the system evaluates whether a treatment event caused a favorable or unfavorable physiologic response.

The seventh row of the classifier output table 600 can correspond to the cuff-pressure classifier. In the illustrated example, the cuff-pressure classifier has output values of 0.25 for Class 1, 0.40 for Class 2, 0.20 for Class 3, 0.10 for Class 4, and 0.05 for Class 5. This row can represent classification information derived from non-invasive blood-pressure measurements. The cuff-pressure classifier can process intermittent systolic pressure, diastolic pressure, mean arterial pressure, cuff-measurement frequency, blood-pressure trends, blood-pressure variability, missing measurement intervals, or differences between cuff-based and arterial-line-based pressure measurements. In some embodiments, the cuff-pressure classifier can be used when an arterial line is unavailable, unreliable, removed, or not selected for use by the processing module.

The eighth row of the classifier output table 600 can correspond to the laboratory-value classifier. In the illustrated example, the laboratory-value classifier has output values of 0.20 for Class 1, 0.45 for Class 2, 0.20 for Class 3, 0.10 for Class 4, and 0.05 for Class 5. This row can indicate that recent laboratory values most strongly support Class 2. The laboratory-value classifier can process measured analytes and laboratory trends, including sodium, glucose, lactate, hemoglobin, hematocrit, platelet count, coagulation values, arterial blood gas values, pH, carbon dioxide, oxygenation values, inflammatory markers, renal-function markers, liver-function markers, electrolyte values, osmolality, or other measured laboratory parameters. In some embodiments, the laboratory-value classifier can also process derived laboratory features, such as rate of change, abnormality duration, trend direction, or deviation from patient-specific baseline values.

The ninth row of the classifier output table 600 can correspond to the medication/pharmacy-event classifier. In the illustrated example, the medication/pharmacy-event classifier has output values of 0.15 for Class 1, 0.40 for Class 2, 0.25 for Class 3, 0.12 for Class 4, and 0.08 for Class 5. This row can indicate that medication or pharmacy-event information supports Class 2 most strongly, with secondary support for Class 3. The medication/pharmacy-event classifier can process medication orders, medication administrations, dose changes, infusion rates, titration events, sedation changes, vasopressor administration, osmotherapy, antiepileptic medication, anticoagulant or reversal-agent administration, analgesic administration, paralytic use, antibiotics, or other medication-related events. The row can capture the relationship between treatment actions and physiologic or neurologic state classification.

The tenth row of the classifier output table 600 can correspond to the ventilation/oxygenation classifier. In the illustrated example, the ventilation/oxygenation classifier has output values of 0.25 for Class 1, 0.35 for Class 2, 0.20 for Class 3, 0.12 for Class 4, and 0.08 for Class 5. This row can indicate that respiratory or oxygenation data supports Class 2 most strongly but also provides information across several classes. The ventilation/oxygenation classifier can process ventilator mode, respiratory rate, tidal volume, minute ventilation, airway pressure, fraction of inspired oxygen, oxygen saturation, end-tidal carbon dioxide, arterial oxygenation, carbon-dioxide values, oxygen-delivery device settings, apnea events, hypoxia events, hypercapnia events, or other respiratory features. In brain-injury monitoring, these respiratory features can be relevant because oxygenation, ventilation, and carbon dioxide levels can affect cerebral perfusion, intracranial pressure, and neurologic recovery.

The eleventh row of the classifier output table 600 can correspond to the treatment-event classifier. In the illustrated example, the treatment-event classifier has output values of 0.20 for Class 1, 0.50 for Class 2, 0.15 for Class 3, 0.10 for Class 4, and 0.05 for Class 5. This row can indicate that the discrete treatment-event information supports Class 2 most strongly. The treatment-event classifier can process information identifying the occurrence, type, timing, duration, dosage, intensity, or sequence of treatment events. Treatment events can include medication administration, medication titration, osmotherapy, cerebrospinal-fluid drainage, ventilator adjustment, blood-pressure intervention, imaging acquisition, surgical intervention, bedside procedure, positioning change, stimulation event, laboratory draw, transfusion, or other clinically relevant intervention. In certain embodiments, the treatment-event classifier can be used to anchor a comparison between pre-event and post-event feature vectors.

The values within each row can be interpreted independently before weighting. For example, the text/clinical-note data classifier may favor Class 2, while the intracranial-pressure classifier may favor Class 1. This disagreement between rows can be preserved in the classifier output table 600 rather than being prematurely collapsed into a single output. Preserving the per-classifier distributions allows the processing module to later determine whether different data sources agree, disagree, or provide complementary information. The system can use such agreement or disagreement to calculate a confidence level, an uncertainty measure, an indeterminate classification, or a recommendation to acquire additional data.

In some embodiments, the classifier output table 600 can be generated for a particular time window. For example, the classifier output table 600 can represent classifier outputs during a pre-treatment time window, during a treatment-event time window, or during a post-treatment time window. The processing module can generate multiple classifier output tables 600 over time and compare corresponding rows, columns, or aggregate outputs. A pre-event classifier output table can be compared with a post-event classifier output table to identify a shift in class distribution, such as a shift from Class 3 toward Class 2, or from Class 2 toward Class 4. The shift can be used to generate a treatment-response vector, a recovery-trajectory update, a deviation flag, or a care-team output.

In some embodiments, the classifier output table 600 can be generated using patient-specific feature vectors. A pre-event feature vector can include features extracted before a treatment event from one or more of the data streams identified in the rows of the table. A post-event feature vector can include corresponding features extracted after the treatment event. The classifiers associated with the rows of the classifier output table 600 can operate on those feature vectors to produce the per-class output values shown in the table. The table 600 can therefore expose how each data-source-specific feature vector contributes to the overall classification analysis.

In certain embodiments, the classifier output table 600 can identify only classifiers having available data for the patient. For example, if ICP data are unavailable for a patient, the ICP classifier row can be omitted, grayed out, marked as unavailable, or assigned a null output. If EEG data are available but have poor signal quality, the EEG row can be included but later weighted downward in a weighted classifier output table. If recent laboratory values are stale, missing, or outside an acceptable recency window, the laboratory-value classifier row can be omitted or flagged for reduced reliability. Thus, the table 600 can be dynamically generated based on patient-data availability and patient-data adequacy.

The classifier output table 600 can also support explainability and model-input transparency. By displaying the separate rows and class-output values, the system can show whether a generated classification was primarily supported by imaging data, physiologic data, laboratory data, medication-event data, treatment-event data, or a combination of those sources. For instance, if Class 2 is the dominant final classification, the care team can inspect the table 600 to determine that the text/clinical-note data classifier, laboratory-value classifier, medication/pharmacy-event classifier, and treatment-event classifier each strongly supported Class 2, while the ICP classifier supported Class 1. The system can then identify the ICP classifier as a potential source of discordance or as a clinically important data stream requiring further review.

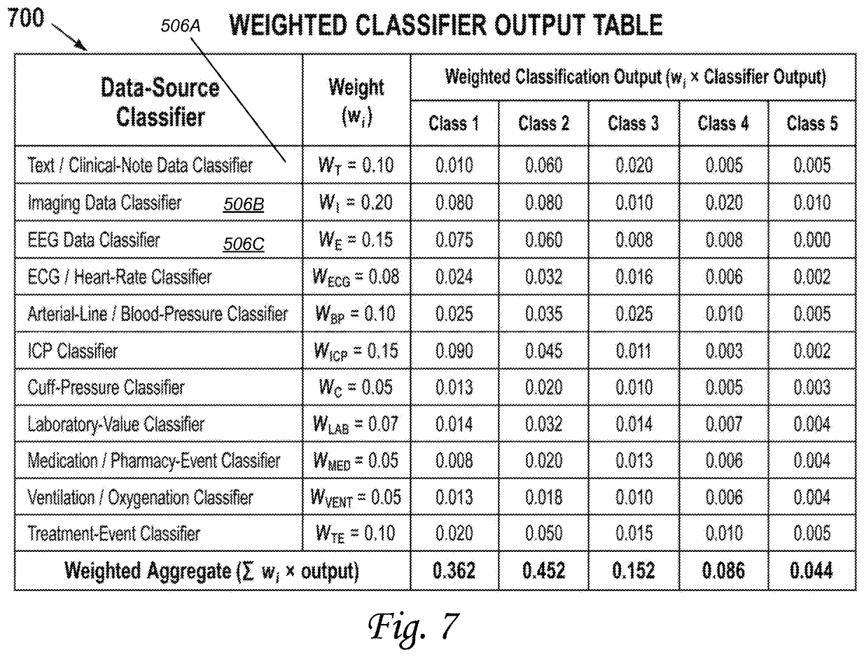

The classifier output table 600 can be used to generate a subsequent weighted classifier output table, such as the table shown in FIG. 7. The output values in FIG. 6 can be multiplied by weighting factors that reflect signal quality, data recency, missing-data status, reliability, patient-specific relevance, classifier confidence, feature-attribution values, clinician configuration, or learned classifier performance. For example, the ICP classifier row can be weighted more heavily when recent, high-quality ICP data are available, while the cuff-pressure classifier row can be weighted less heavily when invasive arterial-line data are available and reliable. Similarly, the EEG classifier row can be weighted downward when EEG artifacts are detected.

The classifier output table 600 is not limited to the exact labels or values shown. The number of classes can be two, three, four, five, or more than five. The classes can be ordinal, categorical, probabilistic, or clinically defined. The data-source classifier rows can be selected based on available equipment, patient condition, institutional workflow, clinician configuration, or model design. Additional classifier rows can include oxygen saturation, cerebral perfusion pressure, end-tidal carbon dioxide, pupillometry, temperature, glucose, seizure burden, sedation depth, neurologic examination findings, fluid balance, renal function, infection markers, or other physiologic, neurologic, laboratory, imaging, or treatment-event features. The specific numeric values shown in FIG. 6 are exemplary and can be replaced by patient-specific values generated by the processing module during operation.

Referring to FIG. 7, there is illustrated one example of a weighted classifier output table 700 generated by the processing module from classifier outputs associated with a plurality of patient-data classifiers. The weighted classifier output table 700 can be generated as an intermediate data structure, a displayed user-interface element, a stored record, a transmitted message, a model-fusion report, or an explainability output. In the illustrated embodiment, the weighted classifier output table 700 includes a left-side data-source classifier column, a weight column, a plurality of weighted classification-output columns, and a weighted aggregate row. The weighted classifier output table 700 can be used to show how outputs from different patient-data classifiers are adjusted and combined to produce an aggregate classification distribution.

The weighted classifier output table 700 includes a table identifier 700. The table identifier 700 can be used in the drawings and specification to refer to the overall weighted classifier-output structure. In this example, the table 700 is labeled as a “Weighted Classifier Output Table,” indicating that at least some values in the table have been modified by weighting factors before being aggregated. The table 700 can therefore represent a fusion stage in which modality-specific classifier outputs, such as those shown in FIG. 6, are adjusted according to reliability, data quality, recency, patient relevance, model confidence, or other weighting criteria before a final or intermediate classification output is generated.

The weighted classifier output table 700 includes a first column labeled “Data-Source Classifier.” The data-source classifier column identifies the respective patient-data classifier, data stream, data modality, or derived-data category associated with each row. In the illustrated example, the data-source classifier column includes a text/clinical-note data classifier, an imaging data classifier, an EEG data classifier, an ECG/heart-rate classifier, an arterial-line/blood-pressure classifier, an intracranial-pressure classifier, a cuff-pressure classifier, a laboratory-value classifier, a medication/pharmacy-event classifier, a ventilation/oxygenation classifier, and a treatment-event classifier. These classifiers correspond to patient-data sources that can be used by the system to evaluate injury severity, brain state, recovery state, treatment response, deviation risk, or other patient-state classifications.

The weighted classifier output table 700 further includes a weight column labeled “Weight” or “Weight (wᵢ).” The weight column can identify a weighting factor applied to the classifier outputs in the corresponding row. In the illustrated example, the text/clinical-note data classifier is associated with a text weight Wₜ, the imaging data classifier is associated with an imaging weight Wᵢ, the EEG data classifier is associated with an EEG weight Wₑ, the ECG/heart-rate classifier is associated with an ECG weight WECG, the arterial-line/blood-pressure classifier is associated with a blood-pressure weight WBP, the intracranial-pressure classifier is associated with an intracranial-pressure weight WICP, the cuff-pressure classifier is associated with a cuff-pressure weight WC, the laboratory-value classifier is associated with a laboratory weight WLAB, the medication/pharmacy-event classifier is associated with a medication weight WMED, the ventilation/oxygenation classifier is associated with a ventilation weight WVENT, and the treatment-event classifier is associated with a treatment-event weight WTE. The weight symbols and values are exemplary and can be replaced by other weighting identifiers, values, or formulas.

Each weighting factor can represent how much the corresponding data-source classifier contributes to a combined classification output. The weighting factor can be predetermined, dynamically calculated, clinician-selected, patient-specific, time-window-specific, classifier-specific, or learned by a machine-learning model. In some embodiments, the weighting factor can be based on signal quality, artifact status, data recency, missing-data status, data completeness, reliability of a sensor, patient-specific relevance of the data source, classifier confidence, model calibration, historical accuracy of the classifier, feature-attribution values, clinician configuration, institutional protocol, or combinations thereof.

For example, an intracranial-pressure weight WICP can be increased when recent, high-quality ICP data are available from an intracranial-pressure sensor and can be decreased when the ICP signal is stale, missing, noisy, or inconsistent with other physiologic data. An EEG weight Wₑ can be increased when continuous EEG data are available and artifact-filtered and can be decreased when the EEG data include motion artifacts, electrode detachment, excessive noise, or insufficient duration. An imaging weight Wᵢ can be increased when recent imaging-derived features are available with acceptable segmentation quality or registration quality and can be decreased when imaging is old, incomplete, artifact-limited, or associated with low segmentation confidence. A cuff-pressure weight WC can be reduced when invasive arterial-line data are available and reliable, or increased when arterial-line data are not available and cuff-pressure measurements are the primary blood-pressure source.

The weighted classifier output table 700 includes a weighted classification-output header extending across a plurality of class columns. In the illustrated embodiment, the weighted classification-output columns are labeled Class 1, Class 2, Class 3, Class 4, and Class 5. The classification classes can represent injury-severity classes, brain-state classes, recovery-state classes, recovery-trajectory classes, treatment-response classes, expected-envelope classes, deviation-risk classes, or another set of patient-state categories. The same class labels can be used in the unweighted classifier output table of FIG. 6 so that outputs from FIG. 6 can be multiplied by row-specific weighting factors and arranged in FIG. 7.

Each cell in the weighted classification-output columns can include a weighted output value generated by applying the weighting factor for a row to the corresponding classifier output value for that class. In one example, a weighted output value can be calculated according to the expression wᵢ×classifier output, where wᵢ is the weighting factor for the corresponding classifier row. Thus, if the text/clinical-note data classifier has an unweighted Class 2 output of 0.60 and a text weight Wₜ of 0.10, the corresponding weighted Class 2 output can be 0.060. If the imaging data classifier has an unweighted Class 1 output of 0.40 and an imaging weight Wᵢ of 0.20, the corresponding weighted Class 1 output can be 0.080. These values are exemplary and can be generated using other weighted, normalized, calibrated, or transformed combinations.

The first row of the weighted classifier output table 700 can correspond to the text/clinical-note data classifier. In the illustrated example, the text/clinical-note data classifier is associated with a text weight Wₜ equal to 0.10. The weighted values in the row can be generated by multiplying the unweighted text/clinical-note classifier outputs by Wₜ. For example, the weighted values can include 0.010 for Class 1, 0.060 for Class 2, 0.020 for Class 3, 0.005 for Class 4, and 0.005 for Class 5. This row can indicate that clinical text supports Class 2 most strongly, but the degree of influence of that support on the combined classification is limited by the applied text weight.

The second row of the weighted classifier output table 700 can correspond to the imaging data classifier. In the illustrated example, the imaging data classifier is associated with an imaging weight Wᵢ equal to 0.20. The weighted imaging values can include 0.080 for Class 1, 0.080 for Class 2, 0.010 for Class 3, 0.020 for Class 4, and 0.010 for Class 5. This row can indicate that imaging-derived features provide approximately equal weighted support for Class 1 and Class 2. The imaging weight can be influenced by factors such as image recency, modality, lesion detectability, segmentation quality, registration quality, image artifacts, or confidence associated with imaging-derived measurements.

The third row of the weighted classifier output table 700 can correspond to the EEG data classifier. In the illustrated example, the EEG data classifier is associated with an EEG weight Wₑ equal to 0.15. The weighted EEG values can include 0.075 for Class 1, 0.060 for Class 2, 0.008 for Class 3, 0.008 for Class 4, and 0.000 for Class 5. This row can indicate that EEG-derived information has meaningful weighted influence on Class 1 and Class 2. The EEG weight can be influenced by EEG signal quality, electrode status, artifact detection, duration of available EEG data, seizure-detection confidence, spectral-feature quality, or neurologic relevance of the EEG features.

The fourth row of the weighted classifier output table 700 can correspond to the ECG/heart-rate classifier. In the illustrated example, the ECG/heart-rate classifier is associated with an ECG weight WECG equal to 0.08. The weighted ECG/heart-rate values can include 0.024 for Class 1, 0.032 for Class 2, 0.016 for Class 3, 0.006 for Class 4, and 0.002 for Class 5.This row can indicate that cardiac-derived features provide support across multiple classes, with Class 2 receiving the greatest weighted support. The ECG weight can be based on cardiac signal quality, heart-rate variability reliability, rhythm-classification confidence, availability of continuous ECG data, or the clinical relevance of cardiac-derived features to the patient's current brain-injury monitoring context.

The fifth row of the weighted classifier output table 700 can correspond to the arterial-line/blood-pressure classifier. In the illustrated example, the arterial-line/blood-pressure classifier is associated with a blood-pressure weight WBP equal to 0.10. The weighted blood-pressure values can include 0.025 for Class 1, 0.035 for Class 2, 0.025 for Class 3, 0.010 for Class 4, and 0.005 for Class 5. This row can indicate that arterial-line or blood-pressure data provides weighted support for Class 2, while also contributing to Classes 1 and 3. The blood-pressure weight can be adjusted based on arterial-line waveform quality, damping status, calibration status, availability of invasive measurements, blood-pressure variability, or agreement between invasive and non-invasive blood-pressure sources.

The sixth row of the weighted classifier output table 700 can correspond to the intracranial-pressure classifier. In the illustrated example, the intracranial-pressure classifier is associated with an intracranial-pressure weight WICP equal to 0.15. The weighted ICP values can include 0.090 for Class 1, 0.045 for Class 2, 0.011 for Class 3, 0.003 for Class 4, and 0.002 for Class 5. This row can indicate that ICP data strongly contributes to Class 1 in the weighted table. The ICP weight can be increased when ICP measurements are recent and reliable and can be decreased when ICP data are missing, stale, noisy, artifact-limited, or unavailable. In some embodiments, the ICP weight can also depend on the clinical context, such as whether the patient has an ICP monitor, whether ICP is a primary monitored variable for the patient, or whether a treatment event was expected to affect intracranial pressure.

The seventh row of the weighted classifier output table 700 can correspond to the cuff-pressure classifier. In the illustrated example, the cuff-pressure classifier is associated with a cuff-pressure weight WC equal to 0.05. The weighted cuff-pressure values can include 0.013 for Class 1, 0.020 for Class 2, 0.010 for Class 3, 0.005 for Class 4, and 0.003 for Class 5. This row can indicate that non-invasive blood-pressure measurements contribute to the combined classification but may be given lower weight than invasive arterial-line measurements when arterial-line data are available. The cuff-pressure weight can be increased when cuff-pressure measurements are the only available blood-pressure source and can be decreased when measurements are infrequent, stale, motion-affected, or inconsistent.

The eighth row of the weighted classifier output table 700 can correspond to the laboratory-value classifier. In the illustrated example, the laboratory-value classifier is associated with a laboratory weight WLAB equal to 0.07. The weighted laboratory values can include 0.014 for Class 1, 0.032 for Class 2, 0.014 for Class 3, 0.007 for Class 4, and 0.004 for Class 5. This row can indicate that laboratory data contributes primarily to Class 2 in the illustrated example. The laboratory weight can be based on laboratory recency, completeness, abnormality magnitude, trend strength, relevance of particular analytes, or confidence that the laboratory values reflect the patient's current physiologic state. For example, older laboratory data can be assigned a lower weight than laboratory data obtained within a current monitoring window.

The ninth row of the weighted classifier output table 700 can correspond to the medication/pharmacy-event classifier. In the illustrated example, the medication/pharmacy-event classifier is associated with a medication weight WMED equal to 0.05. The weighted medication values can include 0.008 for Class 1, 0.020 for Class 2, 0.013 for Class 3, 0.006 for Class 4, and 0.004 for Class 5. This row can indicate that medication-event information contributes to the combined classification, but with a relatively modest weight in the illustrated example. The medication weight can be adjusted based on timing of medication administration, expected onset of action, dose magnitude, titration status, medication class, relevance to the current treatment event, and confidence that the medication event was actually administered rather than merely ordered.

The tenth row of the weighted classifier output table 700 can correspond to the ventilation/oxygenation classifier. In the illustrated example, the ventilation/oxygenation classifier is associated with a ventilation weight WVENT equal to 0.05. The weighted ventilation or oxygenation values can include 0.013 for Class 1, 0.018 for Class 2, 0.010 for Class 3, 0.006 for Class 4, and 0.004 for Class 5. This row can indicate that respiratory information provides weighted support across several classes. The ventilation weight can be based on the availability and reliability of oxygenation data, ventilator settings, arterial blood gas data, end-tidal carbon dioxide data, oxygen saturation data, respiratory artifact status, or the expected effect of oxygenation and ventilation on cerebral physiology.

The eleventh row of the weighted classifier output table 700 can correspond to the treatment-event classifier. In the illustrated example, the treatment-event classifier is associated with a treatment-event weight WTE equal to 0.10. The weighted treatment-event values can include 0.020 for Class 1, 0.050 for Class 2, 0.015 for Class 3, 0.010 for Class 4, and 0.005 for Class 5. This row can indicate that the timing and type of treatment event provides meaningful support for Class 2. The treatment-event weight can be adjusted based on whether the event is a major or minor intervention, the expected physiologic effect of the event, the temporal proximity between the event and the analyzed data window, whether the event is clinician-confirmed, and whether the event corresponds to a treatment being specifically evaluated by the system.

The weighted classifier output table 700 further includes a bottom row labeled “Weighted Aggregate,” “Weighted Aggregate (Σwᵢ×output),” or another equivalent label. The weighted aggregate row can include an aggregate value for each class. Each aggregate value can be generated by summing the weighted output values in the corresponding class column across the classifier rows. In the illustrated example, the weighted aggregate row includes 0.362 for Class 1, 0.452 for Class 2, 0.152 for Class 3, 0.086 for Class 4, and 0.044 for Class 5. These aggregate values can form a combined classification distribution across the classes. In the illustrated example, Class 2 has the highest weighted aggregate value, and the system may identify Class 2 as the most likely classification, subject to confidence, reliability, and threshold rules.

The weighted aggregate row can be used to generate a final classification output, a provisional classification output, a confidence level, an uncertainty level, a deviation flag, a recovery trajectory, a treatment-response output, or a care-team output. For example, if the weighted aggregate for Class 2 exceeds the weighted aggregates for the other classes and satisfies a minimum confidence threshold, the processing module can classify the patient state as Class 2. If the difference between Class 1 and Class 2 is below a margin threshold, the processing module can generate a lower confidence score, an indeterminate output, or a recommendation that additional data be obtained before relying on the classification.

In some embodiments, the weighted aggregate row can be normalized after summation so that the aggregate values sum to one. In other embodiments, the weighted aggregate values can remain unnormalized and can be processed by a calibration module, softmax operation, thresholding operation, Bayesian fusion module, ensemble model, or other aggregation method. The processing module can also compare the weighted aggregate row with a prior weighted aggregate row generated during an earlier time window to determine whether the patient's classification distribution has shifted over time.

The weighted classifier output table 700 can be generated for a pre-event time window, a post-event time window, or a sequence of time-indexed monitoring windows. In one embodiment, the processing module can generate a first weighted classifier output table before a treatment event and a second weighted classifier output table after the treatment event. The processing module can compare the weighted aggregate rows of the first and second tables to determine a change in class distribution. That change can be used to calculate a treatment-response vector, update a recovery trajectory, identify a deviation from an expected recovery envelope, or generate a care-team output. For example, movement of the weighted aggregate distribution toward a favorable recovery class can indicate a positive treatment response, while movement toward an unfavorable deviation class can indicate a negative or concerning response.

The weighted classifier output table 700 can also support model-input transparency and explainability. Because each classifier row retains its own weight and its own weighted class-output values, the system can identify which data streams most influenced the final aggregate classification. For example, the system can determine that the weighted aggregate for Class 2 was strongly influenced by the text/clinical-note data classifier, imaging data classifier, laboratory-value classifier, and treatment-event classifier. The system can also determine that the ICP classifier contributed more strongly to Class 1 than to Class 2, thereby identifying a potentially discordant or clinically important feature set. This information can be presented to a care team as contributing features, influential data sources, confidence information, or individualized care-plan guidance.

In some embodiments, the processing module can use the weights and weighted outputs to generate an adequacy or reliability indication. For example, if high-importance data streams such as ICP or EEG are missing, stale, or low quality, the processing module can reduce the corresponding weights and generate a warning that the classification was based on limited data. If a weighted aggregate output is dominated by low-reliability data sources, the system can generate an indeterminate classification or request updated data. If a high-weight row indicates poor signal quality, the system can recommend checking a sensor, obtaining updated imaging, repeating laboratory testing, or increasing monitoring frequency.

The weighting factors can be determined in several ways. In some embodiments, the weights can be predefined according to a clinical protocol. In other embodiments, the weights can be learned from training data or adjusted over time based on model performance. In still other embodiments, the weights can be determined dynamically for each patient and each time window based on data availability, signal-quality metrics, feature-attribution values, SHAP values, permutation-importance values, attention weights, DICE similarity scores for imaging-derived segmentations, registration-quality scores, missing-data indicators, sensor-status values, or clinician-selected priorities. The same classifier can therefore receive different weights for different patients, different treatment events, or different monitoring periods.

The weighted classifier output table 700 is not limited to the specific rows, weights, or values shown. The number of classifier rows can be increased or decreased based on available patient data. The number of classification columns can be two, three, four, five, or more than five. The weights can be positive, zero, normalized, non-normalized, bounded, learned, rule-based, or time-varying. A classifier row can be assigned a zero weight when its data source is unavailable or unreliable, and the row can be omitted from the aggregation entirely in some embodiments. Additional rows can include oxygen saturation, cerebral perfusion pressure, end-tidal carbon dioxide, pupillometry, temperature, glucose, seizure burden, sedation depth, neurologic examination findings, fluid balance, renal function, infection markers, or other physiologic, neurologic, laboratory, imaging, or treatment-event features.

In this manner, the weighted classifier output table 700 can provide a structured mechanism for combining heterogeneous patient-data classifiers into a clinically interpretable aggregate output. The table 700 can preserve information about individual data-source contributions while also producing an aggregate distribution that can drive downstream actions, including classification of injury severity, classification of brain state, generation of a recovery trajectory, calculation of a treatment-response vector, detection of a deviation from an expected recovery envelope, generation of a confidence level, generation of a care-team output, or generation of an individualized care-plan recommendation.

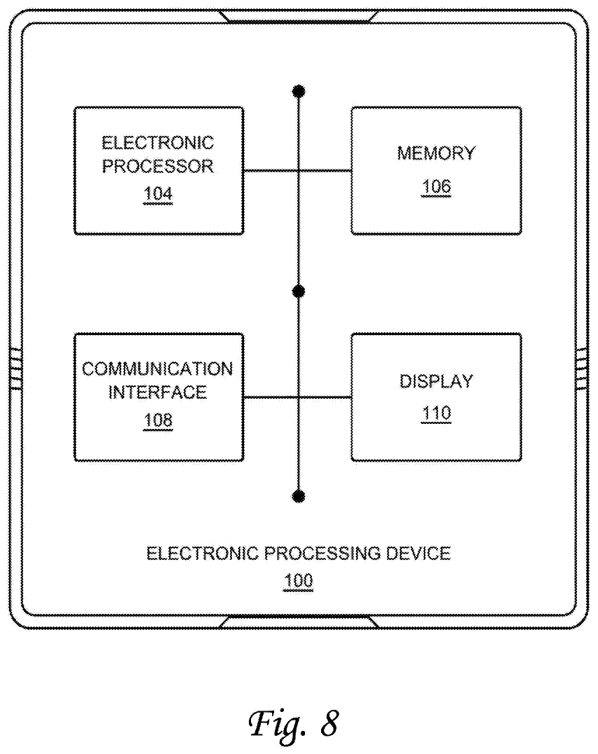

Referring to FIG. 8, there is illustrated one example of a bedside monitoring apparatus 100 configured as an electronic processing device for receiving, processing, storing, and displaying patient information associated with brain-injury monitoring. In an exemplary embodiment, the bedside monitoring apparatus 100 can include an electronic processor 104, a memory 106, a communication interface 108, and a display 110. FIG. 8 can provide a component-level view of the apparatus 100 that may be used within the overall brain-injury treatment-response monitoring system 10 described with respect to FIG. 1 and the bedside monitoring arrangement described with respect to FIG. 2.

The electronic processor 104 can include one or more processors, microprocessors, controllers, digital signal processors, application-specific integrated circuits, field-programmable gate arrays, graphics processing units, neural processing units, or other processing circuitry. The electronic processor 104 can execute instructions stored in the memory 106 to receive patient data, preprocess sensor signals, extract analytic measures, generate brain-state feature vectors, apply signal-quality gates, compare patient data to matched-cohort data, classify treatment response, generate recovery trajectories, control monitoring parameters, and generate outputs for a care team.

In an exemplary embodiment, the electronic processor 104 can receive continuous EEG waveform data from one or more EEG sensors 116 through the communication interface 108. The electronic processor 104 can also receive clinical-event data from the clinical-event input 120, clinical context data from the EHR/clinical data source 121, and cohort-response data from the matched-cohort database 118. The received data can be time-stamped, buffered, transformed, normalized, and associated with a patient record or treatment episode. This arrangement can allow the apparatus 100 to evaluate brain-state changes in relation to treatment events rather than merely collecting physiologic data in isolation.

The memory 106 can include non-transitory computer-readable memory, volatile memory, non-volatile memory, flash memory, solid-state storage, magnetic storage, local cache memory, removable memory, cloud-synchronized memory, or combinations thereof. The memory 106 can store program instructions, raw waveform data, a rolling waveform buffer, patient-specific clinical data, time-stamped treatment-event data, analytic measures, a brain-state feature table 112, signal-quality information, classifier outputs, cohort-response data, expected response envelopes, treatment-response classifications, recovery trajectories, reports, alerts, and user-interface data. The memory 106 can also store settings for monitoring modes, acquisition parameters, processing parameters, confidence thresholds, alert thresholds, and network-communication settings.

The memory 106 can be used to support a technical improvement in the operation of the apparatus 100. For example, the electronic processor 104 can transform EEG waveform samples into stored feature vectors or compressed brain-state signatures and can later perform comparisons using the stored feature vectors without repeatedly reprocessing the underlying raw EEG waveform samples. This can reduce processing burden, reduce latency, reduce memory bandwidth requirements, and allow the apparatus 100 to generate treatment-response outputs during continuous monitoring. The memory 106 can also store cached cohort-response data so that the apparatus 100 can continue generating or updating outputs when communication with a remote server is unavailable or delayed.

The communication interface 108 can include one or more wired or wireless interfaces configured to receive information from sensors, hospital systems, external servers, and care-team devices. For example, the communication interface 108 can include Ethernet circuitry, Wi-Fi circuitry, Bluetooth circuitry, cellular circuitry, serial communication circuitry, USB circuitry, optical communication circuitry, medical-device communication interfaces, HL7 interfaces, FHIR interfaces, application programming interfaces, or other communication circuitry or protocols. The communication interface 108 can be used to receive continuous EEG waveform data, non-EEG physiological data, EHR data, treatment-event data, laboratory data, imaging metadata, medication data, prior-patient cohort data, and model updates.

In an exemplary embodiment, the communication interface 108 can transmit a compressed brain-state signature to a remote cohort server or matched-cohort database 118 without transmitting the full continuous EEG waveform data. The compressed brain-state signature can include a time-indexed sequence of EEG-derived feature vectors, treatment-event metadata, signal-quality flags, artifact flags, medication-state flags, sedation-state flags, and other context. By transmitting a feature-based representation instead of the entire raw waveform, the apparatus 100 can reduce network burden, improve privacy-preserving operation, and support faster cohort matching or model updating. The communication interface 108 can also receive cohort-response data, expected recovery corridors, percentile bands, or updated model parameters from the remote system.

The display 110 can include a screen, touchscreen, monitor, graphical user interface, bedside display, tablet display, workstation display, or other visual output device. The display 110 can present a recovery trajectory, a treatment-response timeline, a recovery index, a treatment-response state, a deviation flag, an expected response envelope, a confidence indicator, signal-quality status, current biosignals, calculated parameters, alerts, settings, and patient-context information. The display 110 can also allow a clinician to review the signals and features that contributed to a treatment-response classification.

The display 110 can provide an advantage over prior approaches that require a clinician to interpret raw EEG traces or disconnected clinical values. Instead of merely displaying raw waveform data, the display 110 can show processed and event-anchored information, such as whether a post-treatment brain-state change is within an expected response envelope for a matched brain-injury cohort. The display 110 can also indicate whether the system output is indeterminate due to signal-quality limitations, missing data, insufficient cohort match, or medication-related confounding. This can give the care team a more reliable and interpretable view of recovery during a treatment period.

In an exemplary embodiment, the electronic processor 104, memory 106, communication interface 108, and display 110 can cooperate to define a bedside technical architecture. The communication interface 108 can receive continuous EEG waveform data and time-stamped clinical-event data. The electronic processor 104 can align the EEG waveform data with an event time, define a pre-event window 140 and post-event window 142, generate pre-event and post-event brain-state feature vectors, and store the feature vectors in memory 106. The electronic processor 104 can then compare a treatment-response vector to an expected response envelope and generate a treatment-response output for presentation on the display 110.

The apparatus 100 can also support signal-quality gating. For example, the electronic processor 104 can evaluate electrode impedance, missing channels, signal saturation, motion artifact, muscle artifact, data dropout, sedation-related confounding, medication-related confounding, or other reliability factors. If the data fails a reliability threshold, the electronic processor 104 can prevent classification, reduce a confidence score, down-weight one or more classifier outputs, or mark a treatment-response state as indeterminate. The memory 106 can store the signal-quality result, and the display 110 can present the reliability status to the care team.

The apparatus 100 can further support adaptive monitoring. The electronic processor 104 can select a monitoring mode based on a recovery trend, a confidence value, a treatment-event type, or comparison with an expected recovery corridor. The selected monitoring mode can change operation of the apparatus 100 by adjusting a sampling rate, EEG channel set, electrode montage, data-transmission frequency, waveform-buffer retention period, feature-extraction interval, classifier-update interval, signal-quality threshold, alert threshold, or cohort-comparison interval. This adaptive control can improve operation of the apparatus 100 by changing acquisition or processing behavior in response to the monitored patient condition.

In some implementations, the apparatus 100 can be embodied as a dedicated bedside monitor, an ICU monitor, a portable medical device, a tablet-based clinical device, a workstation, a server-connected display terminal, a module integrated into an existing patient monitor, a module integrated into an EEG acquisition system, or a distributed combination of these components. The electronic processor 104, memory 106, communication interface 108, and display 110 need not be housed in a single enclosure. For example, the electronic processor 104 can include a local processor at the bedside and a remote processor on a server, the memory 106 can include local and remote storage, and the display 110 can include a bedside display and a remote clinician dashboard.

The apparatus 100 can be configured to operate in a local mode, a networked mode, or a hybrid mode. In the local mode, the apparatus 100 can use locally stored models, locally stored cohort-response data, and locally processed EEG features to generate a treatment-response output. In the networked mode, the apparatus 100 can communicate with matched-cohort database 118 or another server to obtain updated cohort comparisons and expected response envelopes. In the hybrid mode, the apparatus 100 can process raw waveform data locally while transmitting compressed brain-state signatures and treatment-event metadata to a remote system.

FIG. 8 can therefore show that the bedside monitoring apparatus 100 is not merely a generic display terminal. The electronic processor 104, memory 106, communication interface 108, and display 110 can cooperate to provide a specific monitoring architecture for transforming continuous EEG waveform data and clinical-event data into structured brain-state information, quality-gated classifications, matched-cohort comparisons, adaptive monitoring commands, and care-team outputs. This component arrangement can support the broader treatment-response monitoring system 10 and can provide a technical foundation for the recovery and treatment-response workflows shown in the other figures.

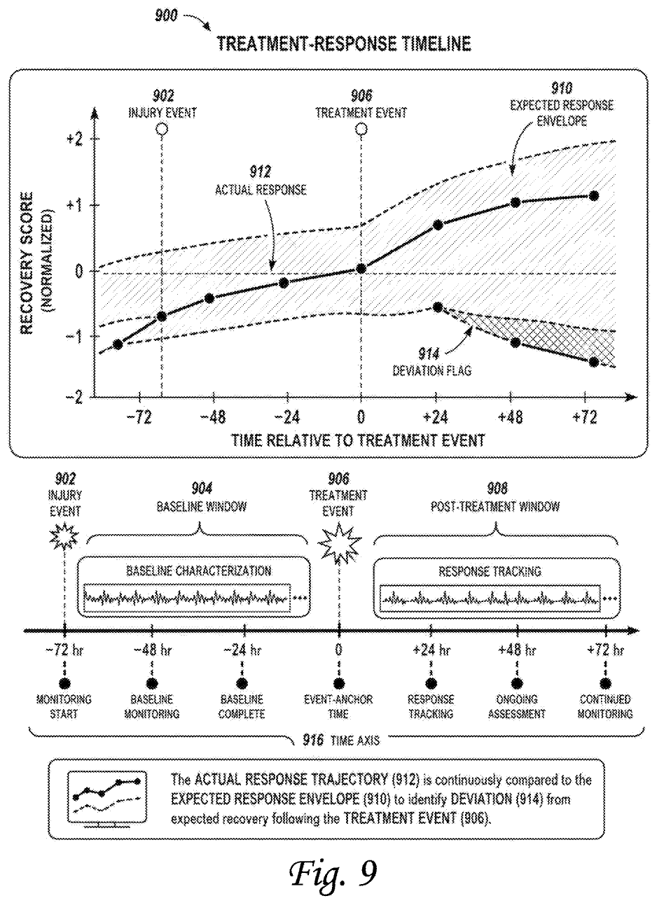

Referring to FIG. 9, there is illustrated one example of a treatment-response timeline 900 showing an injury event 902, a baseline window 904, a treatment event 906, a post-treatment window 908, an expected response envelope 910, an actual response 912, a deviation flag 914, and a time axis 916. FIG. 9 can provide a temporal view of how the brain-injury treatment-response monitoring system 10 can evaluate a patient's recovery or treatment response relative to clinically meaningful time anchors. In an exemplary embodiment, the treatment-response timeline 900 can be generated by the bedside monitoring apparatus 100 and displayed on the display 110 or provided as part of the care-team output 130.

The injury event 902 can represent an initial brain injury, an identified time of symptom onset, a time last known well, a trauma event, a stroke event, a hypoxic event, a hemorrhage event, a seizure event, a surgical event, or another neurologic injury event. The injury event 902 can serve as a temporal reference for selecting clinical context, estimating time from injury onset, choosing a matched brain-injury cohort, and determining which expected recovery model or response envelope should be used. In some implementations, the injury event 902 can be manually entered, automatically extracted from the EHR/clinical data source 121, inferred from clinical documentation, or received from the clinical-event input 120.

The baseline window 904 can represent a temporal window used to characterize the patient's brain state before a treatment event. The baseline window 904 can occur after the injury event 902 and before the treatment event 906. In some embodiments, the baseline window 904 can correspond to the pre-event window 140 described with respect to FIG. 1. The baseline window 904 can be used to generate a baseline brain-state feature vector, a pre-event brain-state signature, a baseline recovery index, or another pre-treatment characterization of the patient. The baseline window 904 can be selected automatically, manually, or according to a monitoring protocol.

In an exemplary embodiment, the baseline window 904 can be selected based on the type of treatment event 906. For a medication event, the baseline window 904 can include a period immediately before administration of the medication. For an intracranial-pressure intervention, the baseline window 904 can include a period before the intervention begins. For thrombectomy, thrombolytic therapy, or another acute stroke intervention, the baseline window 904 can include a period before reperfusion or treatment. For a rehabilitation or stimulation event, the baseline window 904 can include a pre-session characterization period. The baseline window 904 can include minutes, hours, days, or another clinically relevant time interval.

The treatment event 906 can represent a time-stamped intervention or clinical event used as an anchor for evaluating treatment response. The treatment event 906 can include administration of an anti-seizure medication, sedative medication, osmotic therapy, blood-pressure intervention, ventilation adjustment, intracranial-pressure intervention, thrombolytic therapy, thrombectomy, surgery, rehabilitation therapy, neurologic stimulation, medication withdrawal, sedation reduction, or another treatment event. The treatment event 906 can also include a clinical event that may affect interpretation of recovery, such as a seizure, fever, hypotension, hypoxia, elevated intracranial pressure, change in examination status, alarm event, or imaging event.

The post-treatment window 908 can represent a temporal window after the treatment event 906. In some embodiments, the post-treatment window 908 can correspond to the post-event window 142 described with respect to FIG. 1. The post-treatment window 908 can be used to generate a post-event brain-state feature vector, post-treatment brain-state signature, updated recovery index, or other post-treatment characterization. The system 10 can compare features from the baseline window 904 to features from the post-treatment window 908 to determine a treatment-response vector indicating the patient's change across the treatment event 906.

The expected response envelope 910 can represent a normative or matched-cohort-derived range of expected patient response following the treatment event 906. In an exemplary embodiment, the expected response envelope 910 can be generated from prior brain-injury patients selected from the matched-cohort database 118 based on similarity to the current patient. Matching parameters can include injury type, initial severity, time from injury onset, treatment-event type, age, imaging findings, neurological examination findings, medication state, sedation state, physiologic state, and other relevant factors. The expected response envelope 910 can include percentile boundaries, confidence intervals, corridor boundaries, upper and lower expected-response limits, or another comparative range.

The actual response 912 can represent the patient-specific measured or computed recovery response over time. The actual response 912 can be based on EEG-derived features, multimodal classifier outputs, recovery index values, treatment-response vectors, weighted decision-fusion outputs, or other processed data generated by the system 10. In some implementations, the actual response 912 can be updated continuously or periodically as new EEG waveform data, clinical-event data, and clinical context data are received. The actual response 912 can be plotted relative to the expected response envelope 910 so that the care team can visually identify whether the patient is tracking within an expected range or deviating from that range.

The deviation flag 914 can indicate that the actual response 912 has deviated from the expected response envelope 910. The deviation flag 914 can be generated when the actual response 912 crosses an upper boundary, crosses a lower boundary, remains outside the expected response envelope 910 for a defined period, changes direction unexpectedly, fails to improve after the treatment event 906, or satisfies another deviation criterion. In an exemplary embodiment, the deviation flag 914 can be generated only when a confidence threshold and signal-quality threshold are satisfied. If the underlying data is unreliable, the system 10 can instead mark the response as indeterminate rather than generating a deviation flag 914.

The time axis 916 can provide a temporal reference for the treatment-response timeline 900. The time axis 916 can be expressed in minutes, hours, days, treatment epochs, post-injury time, post-treatment time, or another time scale. In the illustrated example, the time axis 916 can show time progressing from the injury event 902 through the baseline window 904, the treatment event 906, and the post-treatment window 908. The treatment event 906 can be assigned an anchor time, such as time zero, so that pre-treatment and post-treatment brain-state changes can be evaluated relative to the event.

In an exemplary embodiment, the system 10 can generate the treatment-response timeline 900 by receiving continuous EEG waveform data through the EEG sensors 116, receiving time-stamped clinical-event data through the clinical-event input 120, and receiving clinical context data from the EHR/clinical data source 121. The electronic processor 104 can identify or receive the injury event 902 and the treatment event 906, define the baseline window 904 and the post-treatment window 908, transform EEG waveform samples from the baseline window 904 into a pre-event brain-state feature vector, and transform EEG waveform samples from the post-treatment window 908 into a post-event brain-state feature vector. The treatment-response engine 122 can then generate a treatment-response vector based on the change between the feature vectors.

The expected response envelope 910 can be generated from the matched-cohort database 118. The matched-cohort database 118 can provide expected recovery or treatment-response information for prior patients having similar injury and treatment characteristics. In some embodiments, the expected response envelope 910 can be generated locally by the bedside monitoring apparatus 100 using cached cohort-response data. In other embodiments, the bedside monitoring apparatus 100 can transmit a compressed brain-state signature and treatment-event metadata to a remote cohort server, and the remote cohort server can return cohort-response data used to generate the expected response envelope 910.

The treatment-response timeline 900 can help distinguish the disclosed system from prior approaches that display only a raw waveform, an isolated health score, or a generic trajectory. FIG. 9 shows that the actual response 912 can be interpreted relative to a treatment event 906 and a matched expected response envelope 910. This can allow the system 10 to evaluate whether a patient's brain state changed in a clinically meaningful time window after a treatment event, rather than merely determining that a patient's data changed at some unspecified time.

The baseline window 904 and the post-treatment window 908 can also support artifact-aware and confounder-aware processing. For example, the signal-quality gate 114 can evaluate whether EEG waveform data in the baseline window 904 and post-treatment window 908 satisfies reliability thresholds. The signal-quality gate 114 can also evaluate whether a medication event, sedation change, electrode problem, missing channel condition, signal saturation, motion artifact, muscle artifact, or other confounder affects the reliability of the actual response 912. If reliability is insufficient, the deviation flag 914 can be suppressed or the response state can be classified as indeterminate.

In an exemplary embodiment, the treatment-response timeline 900 can be used to classify the patient's treatment-response state. If the actual response 912 moves in a favorable direction relative to the expected response envelope 910, the treatment-response state can be classified as improvement. If the actual response 912 moves in an unfavorable direction or crosses outside the expected response envelope 910, the treatment-response state can be classified as deterioration or deviation. If the actual response 912 remains substantially unchanged, the treatment-response state can be classified as plateau. If signal quality, cohort match, or data availability is insufficient, the treatment-response state can be classified as indeterminate.

The treatment-response timeline 900 can further support adaptive control of the bedside monitoring apparatus 100. For example, when the deviation flag 914 is generated, the electronic processor 104 can increase a sampling rate, increase a feature-extraction frequency, increase a data-transmission frequency, expand a waveform-buffer retention period, adjust alert thresholds, request additional data, or select a heightened monitoring mode. When the actual response 912 remains within the expected response envelope 910 for a stability period, the electronic processor 104 can reduce sampling frequency, reduce feature-extraction frequency, reduce data-transmission frequency, or select a lower-intensity monitoring mode. These operations can improve the technical operation of the apparatus 100 by adapting monitoring behavior to the patient's recovery pattern.

The expected response envelope 910 can be generated in different ways. In some implementations, the expected response envelope 910 can be based on time-indexed percentile boundaries derived from the matched brain-injury cohort. In other implementations, the expected response envelope 910 can be based on a model-predicted response curve, a confidence interval around an expected recovery path, a severity-specific outcome band, or an intervention-specific response corridor. The expected response envelope 910 can be updated as new patient data becomes available, as additional cohort data is received, or as the patient's clinical context changes.

The actual response 912 can also be displayed in different forms. The actual response 912 can represent a recovery index, severity probability, fused classifier output, EEG-derived brain-state score, treatment-response vector magnitude, deviation score, normalized recovery value, or another computed value. The actual response 912 can be displayed as a line, dots, trend band, percentile position, heat map, or other graphical element. In some embodiments, multiple actual responses 912 can be displayed, such as separate EEG-derived, imaging-derived, and text-derived response curves, along with a fused response curve.

The deviation flag 914 can be displayed visually and can also generate a care-team output 130. For example, the deviation flag 914 can cause the bedside monitoring apparatus 100 to generate an alert, report, notification, dashboard update, EHR message, or monitoring recommendation. The care-team output 130 can identify the treatment event 906, the time at which deviation occurred, the magnitude of deviation, the confidence level, the contributing features, the signal-quality status, and whether additional review or monitoring is recommended. In some embodiments, the care-team output 130 can further identify one or more features, biosignals, derived measurements, treatment events, medication administrations, physiologic responses, or environmental factors that contributed most strongly to the deviation flag 914 or to the generated recovery trajectory. By identifying the most impactful features or treatment-related factors for the patient, the system can support generation or adjustment of an individualized care plan that prioritizes monitoring, treatment selection, dosing, timing, escalation, de-escalation, or additional diagnostic testing directed to those patient-specific factors. The care-team output 130 can thereby convert the timeline analysis into a structured communication for bedside clinical management and individualized treatment planning.

In certain embodiments, the bedside monitoring apparatus 100 can use the contributing features identified in the care-team output 130 to generate an individualized care-plan recommendation. The individualized care-plan recommendation can include, for example, increased monitoring of a biosignal determined to be highly influential, adjustment of a treatment protocol associated with a favorable or unfavorable treatment-response vector, prioritization of a medication or therapy associated with improved matched-patient outcomes, avoidance of a treatment associated with prior negative deviation, or a recommendation to obtain additional data for a feature having high predicted importance but inadequate current signal quality. The individualized care-plan recommendation can be presented as a clinician-reviewable recommendation rather than an autonomous treatment decision.

FIG. 9 can therefore illustrate a practical and technical way to evaluate brain-injury treatment response. The system 10 can time-align patient waveform data with the injury event 902 and treatment event 906, transform data in the baseline window 904 and post-treatment window 908 into feature-based representations, compare the actual response 912 to the expected response envelope 910, and generate the deviation flag 914 when appropriate. This event-anchored, matched-cohort, quality-gated timeline can provide a foundation for objective treatment-response monitoring during recovery from brain injury.

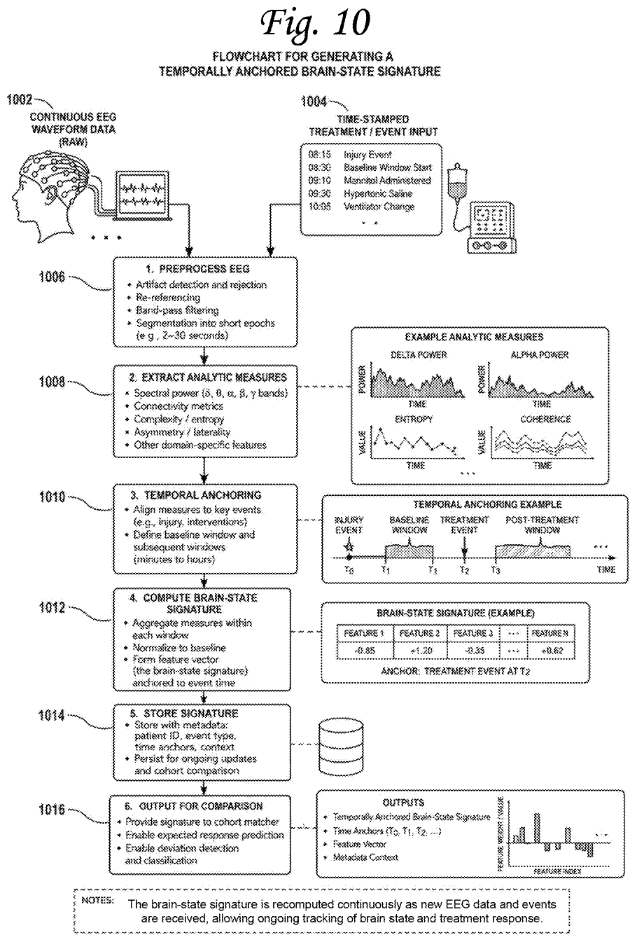

Referring to FIG. 10, there is illustrated one example of a flowchart for generating a temporally anchored brain-state signature based on continuous EEG waveform data 1002 and time-stamped treatment/event input 1004. In an exemplary embodiment, the flowchart can include a preprocess EEG step 1006, an extract analytic measures step 1008, a temporal anchoring step 1010, a compute brain-state signature step 1012, a store signature step 1014, and an output for comparison step 1016. FIG. 10 can focus on the technical transformation of raw or continuous neurophysiological waveform data into a structured brain-state signature that can be stored, compared, transmitted, and used for treatment-response classification.

The continuous EEG waveform data 1002 can include raw EEG waveform samples received from one or more EEG sensors 116 coupled to a patient during a treatment period following a brain injury. The continuous EEG waveform data 1002 can be received continuously, periodically, in response to a clinical event, or according to a selected monitoring mode. In some embodiments, the continuous EEG waveform data 1002 can include multiple EEG channels, a selected electrode montage, time stamps, sampling-rate information, impedance information, channel-status information, and other acquisition metadata. The continuous EEG waveform data 1002 can be stored locally in a rolling waveform buffer of memory 106 before being transformed into analytic measures or feature vectors.

The time-stamped treatment/event input 1004 can identify one or more clinically relevant events that may be used to anchor analysis of the continuous EEG waveform data 1002. The time-stamped treatment/event input 1004 can include an injury event, a medication event, a seizure intervention, an anti-seizure medication administration, a sedative administration or reduction, an osmotic therapy, a blood-pressure intervention, a ventilation change, an intracranial-pressure intervention, thrombolytic therapy, thrombectomy, a procedure, a rehabilitation session, neurologic stimulation, an alarm event, a change in clinical examination, or another event. The time-stamped treatment/event input 1004 can be received from the clinical-event input 120, the EHR/clinical data source 121, a bedside monitor, an infusion pump, a ventilator, a medication-administration record, a clinician interface, or another source.

The preprocess EEG step 1006 can prepare the continuous EEG waveform data 1002 for feature extraction. In an exemplary embodiment, the preprocess EEG step 1006 can include filtering, segmentation, re-referencing, artifact detection, artifact rejection, channel selection, baseline correction, impedance evaluation, missing-channel detection, noise detection, and synchronization of waveform samples with event times. The preprocess EEG step 1006 can also assign signal-quality flags to one or more channels, time windows, or waveform segments. By preprocessing the continuous EEG waveform data 1002 before feature extraction, the system 10 can reduce noise and improve the reliability of downstream treatment-response classification.

The preprocess EEG step 1006 can be performed locally by the bedside monitoring apparatus 100. This local preprocessing can be useful because raw EEG waveform data may be voluminous and may contain artifacts that should be identified before the data is used for recovery analysis or transmitted to a remote system. In some embodiments, the electronic processor 104 can preprocess the continuous EEG waveform data 1002 and can store cleaned, segmented, or annotated waveform portions in the memory 106. The preprocessing can also generate reliability information for use by the signal-quality gate 114.

The extract analytic measures step 1008 can transform the preprocessed EEG data into quantitative analytic measures. In an exemplary embodiment, the analytic measures can include spectral power, delta power, theta power, alpha power, beta power, gamma power, spectral ratios, interhemispheric asymmetry, coherence, connectivity, entropy, rhythmicity, burst-suppression burden, seizure burden, amplitude variability, stimulation reactivity, phase-related measures, time-frequency measures, or other EEG-derived features. The analytic measures can be generated for individual channels, channel groups, hemispheres, brain regions, time windows, or event-aligned epochs.

The extract analytic measures step 1008 can also incorporate non-EEG information when available. For example, the system can associate EEG-derived analytic measures with intracranial pressure values, arterial pressure values, ECG-derived values, oxygenation values, respiratory values, temperature values, medication state, sedation state, neurological examination findings, imaging findings, and other clinical context. The analytic measures can thereby represent brain-state information in context rather than as isolated waveform features. This can be especially useful when a treatment event affects both brain signals and systemic physiology.

The temporal anchoring step 1010 can align the extracted analytic measures with one or more clinically meaningful time references. In an exemplary embodiment, the temporal anchoring step 1010 can align the analytic measures with the time-stamped treatment/event input 1004. The system can define a baseline window, a pre-event temporal window, a post-event temporal window, or a plurality of sequential post-event windows relative to an event time. The temporal anchoring step 1010 can also align analytic measures relative to an injury event, time from symptom onset, time from admission, time from treatment initiation, or time from another clinical event.

The temporal anchoring step 1010 can provide an important technical and clinical distinction over approaches that merely compute a general trend. For example, the same EEG change may have different significance depending on whether the change occurred before a treatment, shortly after a treatment, during a sedative change, after a seizure, or during an intracranial-pressure event. By storing analytic measures in relation to event times, the system 10 can evaluate whether a brain-state change is temporally related to a treatment event and can compare that change to an expected response for similar patients.

The compute brain-state signature step 1012 can aggregate the event-aligned analytic measures into a brain-state signature. In an exemplary embodiment, the brain-state signature can include a time-indexed sequence of EEG-derived feature vectors. The brain-state signature can also include treatment-event metadata, signal-quality flags, artifact flags, medication-state flags, sedation-state flags, patient-context information, injury-type information, time-from-onset information, and confidence information. The brain-state signature can be a compressed representation of the continuous EEG waveform data 1002 and associated clinical context.

The brain-state signature generated at step 1012 can include a pre-event brain-state feature vector and a post-event brain-state feature vector. The pre-event brain-state feature vector can summarize EEG-derived and/or multimodal features during a baseline or pre-treatment window. The post-event brain-state feature vector can summarize EEG-derived and/or multimodal features during a post-treatment window. A treatment-response vector can be computed based on a difference, distance, direction, ratio, normalized change, probability shift, or other comparison between the pre-event and post-event feature vectors. This treatment-response vector can later be compared to an expected intervention-response envelope generated from a matched brain-injury cohort.

The store signature step 1014 can store the brain-state signature in memory 106, in the brain-state feature table 112, in a local database, in a patient record, or in another storage location. The stored signature can be associated with metadata such as patient identification, de-identified patient identifiers, injury type, event type, event time, pre-event window, post-event window, signal-quality status, medication state, sedation state, feature-generation parameters, and model version. The store signature step 1014 can enable the system to perform later comparisons using the stored feature-based representation rather than repeatedly reprocessing the raw EEG waveform samples.

In an exemplary embodiment, the store signature step 1014 can support both local and distributed operation. The bedside monitoring apparatus 100 can store the continuous EEG waveform data 1002 locally while transmitting only the brain-state signature, a compressed brain-state signature, or selected feature information to a remote cohort server or matched-cohort database 118. This can reduce network burden and can allow the remote system to perform cohort selection without receiving the complete continuous EEG waveform data. The stored signature can also allow the bedside monitoring apparatus 100 to continue generating outputs when communication with a remote system is interrupted.

The output for comparison step 1016 can provide the brain-state signature for cohort matching, expected-response prediction, deviation detection, treatment-response classification, recovery trajectory generation, or care-team output generation. In some embodiments, the output for comparison step 1016 can provide the brain-state signature to the matched brain-injury cohort selection engine 1100. In other embodiments, the output for comparison step 1016 can provide the brain-state signature to the treatment-response engine 122, the decision fusion module 508, the EEG data classifier 506C, or another processing module. The output can include the signature itself, a treatment-response vector, confidence data, signal-quality data, or other associated metadata.

The brain-state signature can be compared to prior-patient data from the matched-cohort database 118. For example, a current patient's brain-state signature can be compared to brain-state signatures of prior patients having similar injury type, severity, treatment-event type, time from injury onset, imaging findings, medication state, or physiologic state. The comparison can produce an expected response envelope, a percentile position, a cohort similarity score, a deviation score, an outcome probability, or another recovery-related output. The brain-state signature can thereby serve as a structured technical object that connects continuous EEG waveform processing to matched-cohort treatment-response monitoring.

FIG. 10 can also support signal-quality gating. The preprocess EEG step 1006, extract analytic measures step 1008, temporal anchoring step 1010, and compute brain-state signature step 1012 can each generate or carry forward reliability information. The signal-quality gate 114 can evaluate this reliability information before allowing the brain-state signature to be used for treatment-response classification. If reliability is insufficient, the output for comparison step 1016 can be suppressed, delayed, marked as low confidence, or classified as indeterminate. This can help prevent the system from generating a misleading treatment-response output based on unreliable waveform data or incomplete clinical context.

In some embodiments, the brain-state signature can be generated repeatedly over time. For example, the electronic processor 104 can generate signatures for successive temporal windows, overlapping windows, event-specific windows, or monitoring-protocol-defined windows. The resulting sequence of brain-state signatures can be used to track recovery trajectory, identify deterioration, measure treatment response, update a recovery index, or select an adaptive monitoring mode. The signature-generation workflow can therefore support both continuous recovery monitoring and event-anchored intervention analysis.

The technical flow of FIG. 10 can help differentiate the present invention from prior approaches that rely on manual EEG review, simple display of raw waveforms, isolated frequency calculations, or generic health-status scoring. The workflow can transform continuous EEG waveform data 1002 into a structured, temporally anchored brain-state signature that can be stored, reused, transmitted in compressed form, compared against matched cohorts, and gated based on signal quality. This provides a technical processing improvement because the system can reduce repeated raw-waveform processing, reduce network transmission of raw waveform data, and generate more reliable event-specific treatment-response outputs.

In use, the system 10 can receive continuous EEG waveform data 1002 and time-stamped treatment/event input 1004, preprocess the EEG at step 1006, extract analytic measures at step 1008, temporally anchor the analytic measures at step 1010, compute the brain-state signature at step 1012, store the brain-state signature at step 1014, and provide the brain-state signature for comparison at step 1016. The resulting signature can then be used in later figures for matched-cohort selection, expected-response-envelope generation, treatment-response classification, and clinical-dashboard display.

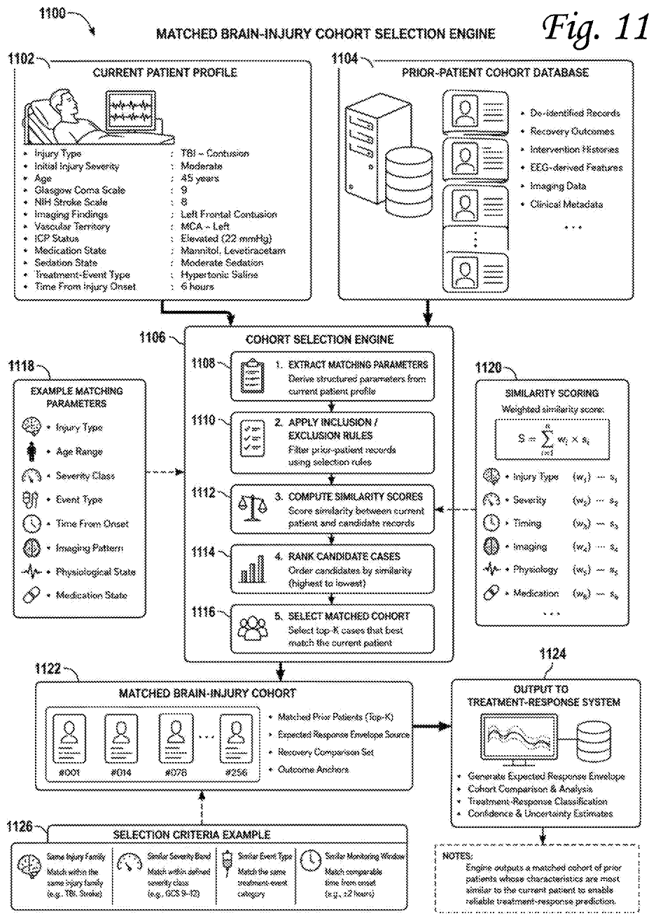

Referring to FIG. 11, there is illustrated one example of a matched brain-injury cohort selection engine 1100 configured to select prior brain-injury patients based on patient-matching parameters. In an exemplary embodiment, the matched brain-injury cohort selection engine 1100 can receive a current patient profile 1102 and access a prior-patient cohort database 1104. A cohort selection engine 1106 can process the current patient profile 1102 and the prior-patient cohort database 1104 using an extract matching parameters step 1108, an apply inclusion/exclusion rules step 1110, a compute similarity scores step 1112, a rank candidate cases step 1114, and a select matched cohort step 1116. The selected cohort can be provided as a matched brain-injury cohort 1122 and can be used to generate output to the treatment-response system 1124.

In an exemplary embodiment, the current patient profile 1102 can include patient-specific information for a brain-injury patient being monitored by the system 10. The current patient profile 1102 can include injury type, injury severity, age, time from injury onset, treatment-event type, imaging findings, physiological state, medication state, sedation state, neurological examination findings, EEG-derived feature values, ICP values, ECG values, laboratory values, comorbidities, and other information relevant to recovery or treatment response. The current patient profile 1102 can be generated from data received by the EEG sensors 116, the clinical-event input 120, the EHR/clinical data source 121, the bedside monitoring apparatus 100, and other patient-monitoring sources.

The prior-patient cohort database 1104 can include historical or prior-patient data associated with prior brain-injury patients. In some embodiments, the prior-patient cohort database 1104 can form part of or communicate with the matched-cohort database 118. The prior-patient cohort database 1104 can include de-identified patient records, injury types, severity scores, treatment-event histories, EEG-derived features, brain-state signatures, imaging data, clinical metadata, physiologic trends, medication information, intervention timing, recovery trajectories, discharge outcomes, functional outcomes, mortality data, rehabilitation outcomes, and other outcome anchors. The prior-patient cohort database 1104 can be stored locally, remotely, in a hospital system, in a cloud environment, in a research network, or in a distributed database architecture.

The cohort selection engine 1106 can select prior-patient records that are sufficiently similar to the current patient profile 1102. The cohort selection engine 1106 can be implemented by the electronic processor 104, by one or more server processors, by the treatment-response engine 122, by a remote cohort server, or by a combination of local and remote processing resources. The cohort selection engine 1106 can improve treatment-response monitoring by avoiding comparisons between the current patient and a generic or poorly matched population. Instead, the current patient can be compared to prior brain-injury patients having similar injury characteristics, treatment-event timing, physiologic context, and recovery conditions.

The extract matching parameters step 1108 can derive structured matching parameters from the current patient profile 1102. In the illustrated embodiment, an example matching parameters region 1118 can identify injury type, age range, severity class, event type, time from onset, imaging pattern, physiological state, and medication state. These parameters can be extracted from raw data, structured clinical records, unstructured notes, imaging reports, EEG-derived features, clinician input, medication records, or other data sources. The extracted matching parameters can be normalized, encoded, scored, binned, or otherwise prepared for cohort comparison.

In an exemplary embodiment, injury type can identify ischemic stroke, hemorrhagic stroke, traumatic brain injury, hypoxic-ischemic injury, subarachnoid hemorrhage, diffuse axonal injury, contusion, seizure-related injury, post-operative brain injury, or another brain-injury category. Age range can account for age-related differences in recovery. Severity class can be based on neurological examination, imaging findings, EEG-derived values, ICP values, multimodal classifier outputs, or other severity indicators. Event type can identify the treatment event being evaluated, such as medication administration, thrombectomy, thrombolysis, blood-pressure intervention, ventilation adjustment, ICP intervention, sedation change, rehabilitation, or stimulation. Time from onset can align the current patient with prior patients at comparable stages of injury evolution.

The apply inclusion/exclusion rules step 1110 can filter candidate prior-patient records from the prior-patient cohort database 1104. The rules can exclude records that do not share a relevant injury family, do not fall within a severity range, lack an applicable treatment event, lack sufficient EEG data, lack outcome data, include incompatible confounders, or fail data-quality criteria. The selection criteria example 1126 can include criteria such as same injury family, similar severity band, similar event type, and similar monitoring window. Additional criteria can include similar age band, similar imaging pattern, similar medication state, similar sedation state, similar physiologic state, or similar time from onset.

The apply inclusion/exclusion rules step 1110 can help avoid misleading comparisons. For example, a patient having traumatic brain injury and elevated intracranial pressure may not be usefully compared against patients having mild ischemic stroke without ICP intervention. Similarly, a patient evaluated after thrombectomy may require a different matched cohort than a patient evaluated after anti-seizure medication administration. The inclusion/exclusion rules can therefore allow the system 10 to generate expected response information based on clinically and physiologically relevant comparators rather than broad averages.

The compute similarity scores step 1112 can calculate similarity between the current patient profile 1102 and each candidate prior-patient record that remains after filtering. The similarity scoring region 1120 can represent a weighted scoring process used to compare patient-matching parameters. The similarity score can be based on exact matches, distance metrics, weighted differences, model-derived similarity, nearest-neighbor techniques, clustering, learned embeddings, propensity scores, or other comparison techniques. The weights used for similarity scoring can differ by injury type, treatment-event type, available data, clinical setting, or monitoring objective.

In an exemplary embodiment, the compute similarity scores step 1112 can assign higher importance to injury type, initial severity, time from injury onset, and treatment-event type. Other parameters, such as age, imaging pattern, physiologic state, medication state, sedation state, and EEG-derived brain-state signature, can also influence the score. The weighting can be configured so that the matched cohort reflects patients who not only have similar clinical diagnoses but also similar brain-state features and treatment contexts. This can improve the relevance of the expected response envelope generated for the current patient.

The rank candidate cases step 1114 can rank candidate prior-patient records according to the similarity scores. The ranking can be performed from most similar to least similar, or by grouping cases into similarity bands. In some embodiments, the rank candidate cases step 1114 can also account for data completeness, outcome availability, recency of data, signal quality, cohort diversity, institutional source, or model confidence. A candidate case with a high similarity score but poor data quality may be ranked below a candidate case with slightly lower similarity but better-quality data.

The select matched cohort step 1116 can select the matched brain-injury cohort 1122 from the ranked candidate cases. The matched brain-injury cohort 1122 can include a fixed number of top-ranked records, all records above a similarity threshold, records within a defined similarity band, records satisfying a minimum data-quality threshold, or a weighted cohort of candidate records. In some embodiments, the matched brain-injury cohort 1122 can be dynamically updated as new patient data becomes available, as the patient moves to a different recovery phase, or as a new treatment event is received.

The matched brain-injury cohort 1122 can be used to generate expected recovery or expected treatment-response information. For example, the matched brain-injury cohort 1122 can be used to generate the expected response envelope 910 shown in FIG. 9. The matched brain-injury cohort 1122 can also be used to generate percentile boundaries, confidence intervals, recovery corridors, outcome probabilities, expected intervention-response curves, severity-transition probabilities, or treatment-response distributions. This information can then be compared to the actual response 912 of the current patient.

The output to treatment-response system 1124 can provide cohort-derived information to another component of the system 10. In an exemplary embodiment, the output to treatment-response system 1124 can include expected response envelope information, cohort comparison data, treatment-response classification data, confidence or uncertainty estimates, cohort-size information, and similarity-score summaries. The output to treatment-response system 1124 can be provided to the treatment-response engine 122, the decision fusion module 508, the display 110, the care-team output 130, or another component.

The matched brain-injury cohort selection engine 1100 can also support confidence estimation. For example, when the selected matched brain-injury cohort 1122 includes many high-similarity prior patients with complete data, the system can assign a higher confidence to the expected response envelope. When the matched cohort is small, poorly matched, missing key variables, or associated with inconsistent outcomes, the system can assign a lower confidence or classify the treatment response as indeterminate. This can provide a reliability-aware output rather than forcing a definitive classification from weak comparator data.

In some embodiments, the cohort selection engine 1106 can use the brain-state signature generated in FIG. 10 as an input to cohort selection. A current patient's time-indexed EEG-derived feature vectors, signal-quality flags, medication-state flags, and treatment-event metadata can be compared with stored signatures of prior patients in the prior-patient cohort database 1104. This allows matching to be based not merely on diagnosis codes or demographic data, but also on the patient's measured neurophysiological state and the timing of the clinical event being evaluated.

The cohort selection engine 1106 can also incorporate multimodal classifier outputs from FIGS. 5, 6, and 7. For example, a fused severity distribution generated by the weighted classifier-output table 700 can be used as a severity parameter or similarity feature. The outputs of the text data classifier 506A, imaging data classifier 506B, and EEG data classifier 506C can each contribute to cohort selection. This can allow the system 10 to select a matched brain-injury cohort 1122 using a more complete patient profile than would be available from one data stream alone.

In an exemplary embodiment, the matched brain-injury cohort selection engine 1100 can be event-specific. The cohort selected for a patient after an anti-seizure medication may differ from the cohort selected for the same patient after a ventilation adjustment, thrombectomy, or rehabilitation event. The event-specific matching can allow the expected response envelope to reflect how similar patients responded to similar interventions at similar stages of recovery. This can provide a more tailored treatment-response comparison than approaches that compare a patient to a general population or to all prior patients having the same diagnosis.

The matched brain-injury cohort selection engine 1100 can also be configured to protect patient privacy and reduce network burden. For example, the bedside monitoring apparatus 100 can transmit a compressed brain-state signature and treatment-event metadata to a remote cohort server without transmitting continuous raw EEG waveform data. The remote server can use the compressed signature and metadata to perform the cohort selection. The server can then return cohort-response data, expected response envelope data, or similarity summaries to the bedside monitoring apparatus 100. The raw EEG waveform data can remain stored locally at the bedside monitoring apparatus 100.

FIG. 11 can help distinguish the present system from prior approaches that provide generalized health trajectories, generic comparative displays, propensity score matching or unfiltered population comparisons. The illustrated matched brain-injury cohort selection engine 1100 can select prior patients based on brain-injury-specific parameters, treatment-event-specific parameters, time-from-onset information, imaging context, physiologic state, medication state, and neurophysiological features. The output can therefore provide an expected response that is specifically tailored to the current patient's injury and treatment context.

The cohort-selection process can also be used to improve operation of the monitoring system. If the output to treatment-response system 1124 indicates a weak cohort match, the electronic processor 104 can increase monitoring intensity, request additional data, adjust a classifier threshold, delay classification, or display a lower-confidence indication. If the matched cohort is strong and the patient remains within an expected response envelope, the system can reduce data-transmission frequency or monitoring intensity during a stability period. The cohort-selection result can therefore influence both the clinical output and the technical operation of the system 10.

In use, the matched brain-injury cohort selection engine 1100 can receive the current patient profile 1102, access the prior-patient cohort database 1104, extract matching parameters at step 1108, apply inclusion/exclusion rules at step 1110, compute similarity scores at step 1112, rank candidate cases at step 1114, select the matched cohort at step 1116, and provide the matched brain-injury cohort 1122 or output to treatment-response system 1124. The matched brain-injury cohort 1122 can then be used to generate expected response data for comparing the current patient's actual response and for supporting treatment-response classification.

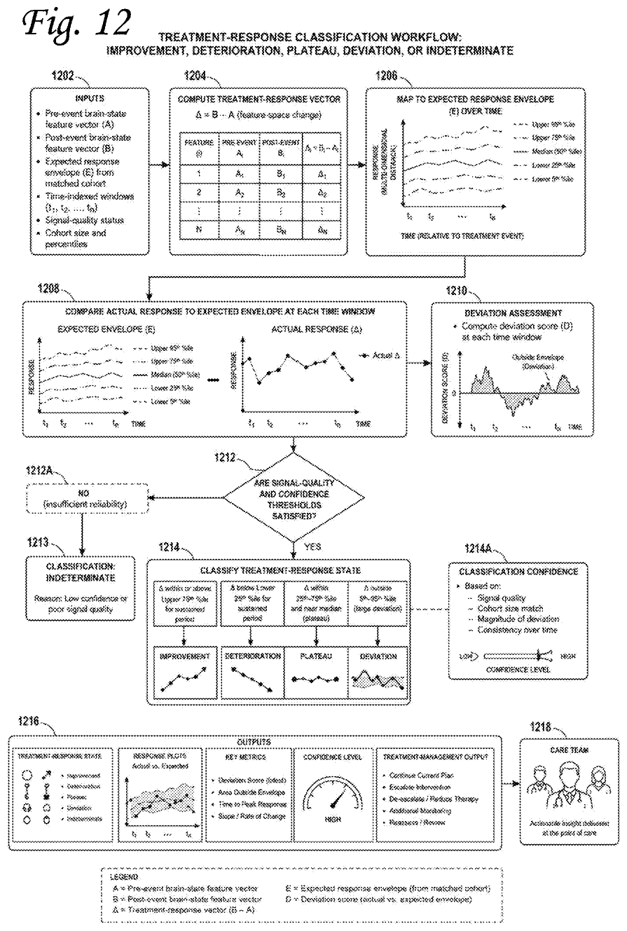

Referring to FIG. 12, there is illustrated one example of a treatment-response classification workflow 1200 configured to classify a treatment-response state of a brain-injury patient. In an exemplary embodiment, the treatment-response classification workflow 1200 can receive classification inputs 1202, compute a treatment-response vector at a treatment-response vector computation block 1204, map expected-response-envelope data at an expected-response-envelope mapping block 1206, compare an actual response to an expected response at an actual-versus-expected comparison block 1208, assess deviation at a deviation assessment block 1210, evaluate signal quality and confidence at a signal-quality/confidence decision 1212, and generate one or more classification outputs 1216 for a care team 1218. The workflow 1200 can be implemented by the bedside monitoring apparatus 100, the electronic processor 104, the memory 106, the signal-quality gate 114, the treatment-response engine 122, the output interface 124, or a combination thereof.

The classification inputs 1202 can include patient-specific data and cohort-derived data used to classify response to a treatment event. In the illustrated example, the classification inputs 1202 can include a pre-event feature vector, a post-event feature vector, an expected response envelope, time-indexed windows, signal-quality status, cohort size, and percentiles. The pre-event feature vector can be generated from EEG waveform data and other patient data acquired during a pre-event or baseline window, such as the baseline window 904 or pre-event window 140. The post-event feature vector can be generated from EEG waveform data and other patient data acquired during a post-treatment window, such as the post-treatment window 908 or post-event window 142. The expected response envelope can be generated from a matched brain-injury cohort, such as the matched brain-injury cohort 1122.

In an exemplary embodiment, the classification inputs 1202 can also include injury type, initial injury severity, time from injury onset, treatment-event type, medication state, sedation state, imaging findings, neurological examination findings, intracranial pressure values, arterial pressure values, ECG values, laboratory values, and other clinical context. These inputs can allow the treatment-response classification workflow 1200 to determine whether a patient's apparent brain-state change is likely attributable to recovery, treatment response, deterioration, artifact, sedation, or another confounding condition. This differs from approaches that treat a physiologic trend as meaningful without evaluating the context in which the trend occurred.

The treatment-response vector computation block 1204 can compute a treatment-response vector based on a difference between the pre-event feature vector and the post-event feature vector. The treatment-response vector can represent a change in feature space across a treatment event. For example, the vector can include changes in spectral power, asymmetry, entropy, coherence, burst-suppression burden, seizure burden, amplitude variability, stimulation reactivity, ICP-derived values, ECG-derived values, or other features. The treatment-response vector can be computed as a subtraction, ratio, normalized difference, distance, angle, probability shift, classifier-distribution shift, or another measure of change between pre-event and post-event brain-state representations.

The treatment-response vector computation block 1204 can provide a technical transformation of continuous waveform data into a structured response measure. Instead of requiring a clinician to manually compare pre-treatment and post-treatment EEG waveforms, the system 10 can transform those waveform segments into feature vectors and compute a repeatable treatment-response vector. The treatment-response vector can be stored in memory 106, added to the brain-state feature table 112, provided to the treatment-response engine 122, or transmitted as part of a compressed brain-state signature.

The expected-response-envelope mapping block 1206 can map expected response data over time. In an exemplary embodiment, the expected-response-envelope mapping block 1206 can receive matched-cohort data from the matched-cohort database 118 or the matched brain-injury cohort selection engine 1100. The expected-response-envelope mapping block 1206 can generate percentile bands, confidence intervals, upper and lower expected-response boundaries, recovery corridors, model-predicted response curves, or other expected response information. The mapped expected response can be time-indexed relative to a treatment event so that the patient's actual response can be compared at clinically meaningful post-event windows.

The expected-response-envelope mapping block 1206 can be configured differently for different injury types or treatment-event types. For example, an expected response after an anti-seizure medication may have different time characteristics than an expected response after a thrombectomy, a sedation change, a ventilation adjustment, an intracranial-pressure intervention, or a rehabilitation session. The workflow 1200 can therefore map expected response data using treatment-event-specific timing rather than relying on a single generic recovery curve.

The actual-versus-expected comparison block 1208 can compare the patient's actual treatment-response vector or actual response trajectory to the expected response envelope. The comparison can be performed at one time window, across multiple time-indexed windows, or continuously as new data becomes available. The actual-versus-expected comparison block 1208 can determine whether the patient response lies within the expected response envelope, above the expected response envelope, below the expected response envelope, trending toward a boundary, or persistently outside the expected response envelope.

In an exemplary embodiment, the actual-versus-expected comparison block 1208 can use a percentile-based comparison. For example, the patient's actual response may be classified as within expected range when it falls between selected percentile boundaries of the matched cohort. The patient's actual response may be classified as deviation when it falls outside those boundaries for a threshold duration or across multiple time windows. The comparison can also use statistical distance, z-score, Mahalanobis distance, similarity score, trajectory divergence, slope comparison, or another quantitative comparison.

The deviation assessment block 1210 can compute a deviation score at one or more time windows. The deviation score can represent a magnitude, duration, direction, or consistency of deviation between the actual response and the expected response envelope. The deviation assessment block 1210 can evaluate whether deviation is favorable, unfavorable, sustained, transient, worsening, improving, or uncertain. In some embodiments, the deviation assessment block 1210 can distinguish a beneficial deviation, such as recovery better than expected, from an adverse deviation, such as deterioration or failure to respond. This allows for changes in treatment to optimize recovery leveraging treatments with beneficial deviations.

The deviation assessment block 1210 can also support the deviation flag 914 shown in FIG. 9. For example, when the deviation score satisfies a deviation criterion, the system 10 can generate the deviation flag 914, produce a care-team output 130, adjust monitoring-system operation, or request additional data. The deviation criterion can include crossing an expected response boundary, remaining outside a boundary for a defined time, deviating across two or more consecutive windows, worsening after a treatment event, failing to improve within an expected period, or satisfying another treatment-response rule.

The signal-quality/confidence decision 1212 can determine whether the data is reliable enough to support a treatment-response classification. In an exemplary embodiment, the signal-quality/confidence decision 1212 can evaluate signal-quality status, artifact flags, electrode impedance, missing-channel conditions, signal saturation, motion artifact, muscle artifact, data dropout, sedation-related confounding, medication-related confounding, cohort size, cohort similarity, percentile stability, model confidence, and consistency of the patient response over time. The signal-quality/confidence decision 1212 can be implemented by or in cooperation with the signal-quality gate 114.

When the signal-quality/confidence decision 1212 determines that signal quality or confidence is insufficient, the workflow 1200 can follow an insufficient reliability branch 1212A to an indeterminate classification 1213. The indeterminate classification 1213 can indicate that the system 10 does not have enough reliable information to classify the treatment-response state as improvement, deterioration, plateau, or deviation. This reliability gate can be important in brain-injury monitoring because EEG and other physiologic signals can be affected by artifact, missing channels, procedures, medication state, and patient movement. The indeterminate classification 1213 can prevent the system 10 from presenting an overconfident output when the underlying information is unreliable.

When the signal-quality/confidence decision 1212 determines that the data satisfies reliability and confidence criteria, the workflow 1200 can proceed to the treatment-response classification module 1214. The treatment-response classification module 1214 can classify the patient's treatment-response state based on the actual response, expected response envelope, deviation score, treatment-response vector, and clinical context. The treatment-response state can include improvement, deterioration, plateau, deviation, or indeterminate. Improvement can indicate that the patient's brain-state features are moving in a favorable direction. Deterioration can indicate an unfavorable change. Plateau can indicate little or no meaningful change. Deviation can indicate that the actual response differs from the expected response envelope in a way that satisfies a deviation criterion.

In an exemplary embodiment, the treatment-response classification module 1214 can use thresholds, rules, classifiers, weighted models, decision trees, neural networks, Bayesian models, ensemble models, or other classification logic. The treatment-response classification module 1214 can use different logic for different injury types, treatment-event types, monitoring windows, or levels of data availability. For example, the system may classify response to an anti-seizure medication using seizure burden and rhythmicity features, while classifying response to an intracranial-pressure intervention using EEG slowing, ICP trends, perfusion-related signals, and recovery-envelope data.

The classification confidence module 1214A can determine a confidence level for the treatment-response classification. The classification confidence can be based on signal quality, cohort-size match, magnitude of deviation, consistency over time, model certainty, availability of multimodal data, concordance between classifiers, or other reliability measures. For example, confidence may be high when EEG quality is good, the matched cohort is large and similar, the actual response consistently deviates from the expected response envelope, and text, imaging, and biosignal classifiers agree. Confidence may be lower when data is sparse, the cohort match is weak, or modalities provide conflicting information.

The classification outputs 1216 can include a treatment-response state, response plots, key metrics, a confidence level, and a treatment-management output. The treatment-response state can identify improvement, deterioration, plateau, deviation, or indeterminate. The response plots can show the actual response relative to an expected response envelope, percentile bands, or time-indexed windows. The key metrics can identify a deviation score, recovery index, treatment-response vector magnitude, feature changes, signal-quality score, cohort size, or other supporting values. The confidence level can indicate the reliability of the classification. The treatment-management output can include a dashboard update, alert, report, monitoring-frequency recommendation, escalation indication, de-escalation indication, intervention-effectiveness indication, or other care-team-facing information.

The care team 1218 can receive the classification outputs 1216 through a bedside display, remote dashboard, electronic health record, notification system, report, or other output interface. The care team 1218 can review the classification outputs 1216 to understand whether the patient is improving, deteriorating, plateauing, deviating from expected recovery, or indeterminate. The care team 1218 can also review the confidence level, contributing features, signal-quality status, and response plots to determine whether the output is sufficiently reliable for clinical consideration.

FIG. 12 can also support adaptive monitoring control. For example, if the treatment-response classification module 1214 classifies the patient as deteriorating or deviating and the classification confidence module 1214A indicates sufficient confidence, the electronic processor 104 can select a heightened monitoring mode. The heightened monitoring mode can increase sampling rate, increase feature-extraction frequency, extend waveform-buffer retention, increase data-transmission frequency, lower alert thresholds, or increase cohort-comparison frequency. If the classification outputs 1216 indicate stability within an expected range for a stability period, the system 10 can select a reduced monitoring mode to conserve processing resources, network bandwidth, or storage.

The treatment-response classification workflow 1200 can provide a specific technical improvement because it does not merely display a recovery trajectory or a raw signal. The workflow 1200 can compute a treatment-response vector, map cohort-derived expected response information, perform actual-versus-expected comparison, assess deviation, apply signal-quality and confidence gating, and generate classification outputs only when reliability criteria are satisfied. This can help address the practical problem of converting complex, noisy, continuous EEG and clinical data into a structured, reliable, event-anchored treatment-response output.

In use, the classification inputs 1202 can be received after the system 10 generates pre-event and post-event feature vectors and obtains an expected response envelope from a matched cohort. The treatment-response vector computation block 1204 can compute the patient's change across the treatment event. The expected-response-envelope mapping block 1206 can map the expected response over time. The actual-versus-expected comparison block 1208 can determine how the patient's response compares to the expected response. The deviation assessment block 1210 can determine whether the comparison indicates meaningful deviation. The signal-quality/confidence decision 1212 can determine whether the result is reliable. The treatment-response classification module 1214 and classification confidence module 1214A can generate the classification outputs 1216 for the care team 1218.

The workflow 1200 can support robust treatment-response monitoring by using event-anchored vector computation, matched-cohort expected-envelope mapping, reliability-gated classification, and structured care-team output. In some embodiments, the workflow 1200 can classify a patient response based on deviation across multiple time-indexed windows, a minimum number of matched prior patient records, a confidence threshold associated with the classification, or a reliability threshold associated with one or more input signals. When the reliability threshold is not satisfied, the workflow 1200 can generate an indeterminate classification, request additional patient data, increase monitoring frequency, suppress or delay a treatment-response classification, or generate a care-team output indicating that the current data are insufficient for reliable classification. In certain embodiments, the workflow 1200 can adapt one or more monitoring parameters based on the classification output, including changing a sampling interval, selecting additional biosignals for monitoring, requesting updated imaging or laboratory data, or generating a recommendation for clinician review.

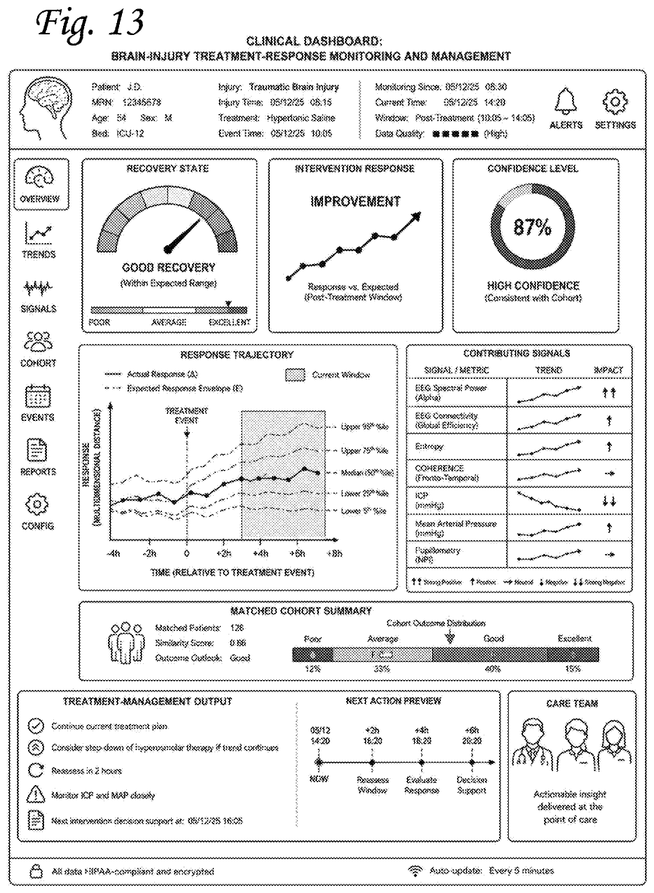

Referring to FIG. 13, there is illustrated one example of a clinical dashboard 1300 configured to present brain-injury recovery information, treatment-response information, confidence information, contributing signals, and a treatment-management output. In an exemplary embodiment, the clinical dashboard 1300 can be generated by the bedside monitoring apparatus 100, the electronic processor 104, the memory 106, the treatment-response engine 122, the output interface 124, and/or a remote system in communication with the bedside monitoring apparatus 100. The clinical dashboard 1300 can provide a care-team-facing interface for presenting the processed results of continuous EEG monitoring, clinical-event anchoring, matched-cohort comparison, and signal-quality/confidence gating.

The clinical dashboard 1300 can include a patient status header 1302. The patient status header 1302 can present identifying or contextual information for the monitored patient, such as a bed identifier, patient identifier, time from injury onset, injury type, treatment period, current monitoring mode, last updated time, or other patient-status information. In some embodiments, the patient status header 1302 can also indicate whether the displayed output is based on live data, delayed data, cached cohort-response data, or data received from a remote matched-cohort server. This can allow the care team to understand the context and freshness of the displayed recovery information.

The clinical dashboard 1300 can include a recovery-state panel 1304. The recovery-state panel 1304 can display a current recovery state or treatment-response state, such as improvement, deterioration, plateau, deviation, or indeterminate. In an exemplary embodiment, the recovery-state panel 1304 can display a recovery index, severity class, fused classifier output, percentile position, recovery score, or another computed recovery-state value. The recovery-state panel 1304 can be generated from EEG-derived features, clinical context, imaging features, text-derived clinical information, and matched-cohort comparison data.

The clinical dashboard 1300 can include an intervention-response panel 1306. The intervention-response panel 1306 can identify a treatment event and can present how the patient's brain-state information changed after the treatment event. For example, the intervention-response panel 1306 can identify a medication event, blood-pressure intervention, ventilation adjustment, intracranial-pressure intervention, thrombectomy, thrombolytic therapy, sedation change, seizure intervention, rehabilitation event, neurologic stimulation event, or another clinical event. The intervention-response panel 1306 can show whether the patient's post-event response is favorable, unfavorable, expected, unexpected, or indeterminate.

In an exemplary embodiment, the clinical dashboard 1300 can include a confidence panel 1308. The confidence panel 1308 can present a confidence level, reliability indicator, signal-quality score, cohort-match score, model-confidence score, or other confidence information associated with the displayed recovery or treatment-response output. The confidence panel 1308 can help prevent an overconfident interpretation of the output when the underlying data is incomplete, noisy, poorly matched, or confounded. For example, the confidence panel 1308 can indicate low confidence when EEG signal quality is poor, when cohort size is small, when the matched cohort is weak, when clinical-event timing is uncertain, or when sedation state confounds interpretation.

The clinical dashboard 1300 can include a trajectory display 1310. The trajectory display 1310 can present a patient's actual response relative to an expected response envelope. In some embodiments, the trajectory display 1310 can correspond to or incorporate the treatment-response timeline 900, expected response envelope 910, actual response 912, and deviation flag 914. The trajectory display 1310 can show changes over time, such as changes in recovery index, severity class, brain-state signature, treatment-response vector, or fused classifier output. The trajectory display 1310 can be anchored to an injury event, treatment event, baseline window, or post-treatment window.

The trajectory display 1310 can be used to show whether the patient is within a matched-cohort expected recovery corridor. The expected recovery corridor can be generated using the matched brain-injury cohort 1122 selected by the matched brain-injury cohort selection engine 1100. The actual patient response can be generated from EEG-derived feature vectors, multimodal classifier outputs, or treatment-response vectors. When the actual response moves outside an expected response envelope, the clinical dashboard 1300 can display a deviation indicator, alert, or status change.

The clinical dashboard 1300 can include a contributing-signals panel 1312. The contributing-signals panel 1312 can identify data sources used to generate the current recovery or treatment-response output. The contributing signals can include EEG, ICP, ECG, arterial pressure, pulse oximetry, respiratory data, NIRS, imaging data, medication data, laboratory values, neurological examination findings, clinical notes, treatment-event data, or other clinical context. In an exemplary embodiment, the contributing-signals panel 1312 can indicate which signals are active, missing, unreliable, down-weighted, excluded, or flagged for artifact.

The contributing-signals panel 1312 can provide transparency into the basis for the treatment-response classification. For example, if the clinical dashboard 1300 indicates deterioration, the contributing-signals panel 1312 can show whether the deterioration was driven by increased seizure burden, increased EEG slowing, increased interhemispheric asymmetry, elevated intracranial pressure, worsening oxygenation, medication state, or another contributing factor. This can help distinguish the disclosed system from approaches that present a single score without explaining which patient data contributed to the output.

The clinical dashboard 1300 can include a treatment-management output panel 1314. The treatment-management output panel 1314 can present an output generated by the treatment-response engine 122 or output interface 124. The treatment-management output can include an alert, monitoring recommendation, escalation indication, de-escalation indication, intervention-effectiveness indication, deviation warning, request for additional data, signal-quality warning, or other care-team-facing output. In some embodiments, the treatment-management output panel 1314 can identify the treatment event, the treatment-response state, the confidence level, the deviation metric, and one or more contributing signals.

In an exemplary embodiment, the treatment-management output panel 1314 can display an indication that monitoring intensity should be increased or reduced. For example, when the patient is deteriorating or deviating from expected recovery with sufficient confidence, the panel 1314 can indicate that a heightened monitoring mode has been selected or recommended. When the patient remains within an expected response envelope for a stability period, the panel 1314 can indicate that a reduced monitoring mode has been selected or that monitoring parameters have been adjusted. The system can thereby provide not only clinical information but also a technical control output that changes operation of the monitoring apparatus 100.

The clinical dashboard 1300 can include a feature-summary panel 1316. The feature-summary panel 1316 can present selected analytic measures, feature changes, treatment-response vector components, classifier probabilities, weighted classifier outputs, deviation scores, percentile ranks, or other quantitative information. The feature-summary panel 1316 can allow the care team to review the underlying values supporting the recovery-state panel 1304 and intervention-response panel 1306. In some embodiments, the feature-summary panel 1316 can be linked to or generated from the brain-state feature table 112.

The clinical dashboard 1300 can include a signal-quality panel 1318. The signal-quality panel 1318 can present signal-quality flags, artifact status, electrode impedance status, missing-channel status, motion artifact status, muscle artifact status, data-dropout status, medication-confounder status, sedation-confounder status, and other reliability information. In an exemplary embodiment, the signal-quality panel 1318 can receive outputs from the signal-quality gate 114. If the signal-quality gate 114 determines that the data fails a reliability threshold, the clinical dashboard 1300 can display an indeterminate state or can identify which signal-quality condition prevented classification.

The clinical dashboard 1300 can include a cohort-comparison panel 1320. The cohort-comparison panel 1320 can present information about the matched cohort used for the expected recovery or expected response comparison. The cohort-comparison panel 1320 can identify a cohort size, cohort similarity score, matching parameters, percentile boundaries, expected response envelope, time-window alignment, or other cohort-derived information. This can help the care team understand whether the expected response envelope is based on a strong match or a weaker match. A weak match can be associated with a lower confidence indicator or an indeterminate output.

In an exemplary embodiment, the cohort-comparison panel 1320 can show that the matched cohort was selected based on injury type, initial injury severity, time from onset, treatment-event type, age range, imaging findings, neurological examination findings, physiologic state, medication state, sedation state, and EEG-derived brain-state signature. The cohort-comparison panel 1320 can thereby show that the patient is not being compared to a generic population but instead to prior brain-injury patients having similar recovery and treatment-response context.

The clinical dashboard 1300 can also support user interaction. For example, a clinician can select an alert region, trajectory region, signal region, cohort region, or treatment-event region to view additional details. The dashboard can allow the clinician to review raw or processed EEG segments, inspect calculated parameters, confirm or edit treatment-event timing, view cohort-matching criteria, inspect signal-quality flags, export a report, or open a related EHR record. The dashboard can be displayed at the bedside, at a clinician workstation, on a tablet, on a mobile device, in an EHR interface, or in a remote monitoring center.

The clinical dashboard 1300 can be updated continuously, periodically, or in response to events. For example, the dashboard can update when new EEG waveform data is received, when a new treatment event is entered, when a post-treatment window becomes available, when the matched cohort is refreshed, when signal quality changes, when a classifier output changes, or when a monitoring mode is changed. In some embodiments, the dashboard can include a last-updated indicator so the care team can determine whether the output reflects current patient data.

The clinical dashboard 1300 can help overcome limitations of prior approaches that require clinicians to separately interpret raw EEG waveforms, review clinical notes, inspect imaging, evaluate vital signs, and mentally compare the patient to an expected recovery path. The dashboard can integrate those data sources into a structured presentation that identifies recovery state, intervention response, confidence level, contributing signals, and treatment-management output. This integrated display can reduce cognitive burden and provide an event-anchored view of how the patient is responding during a treatment period.

In an exemplary embodiment, the clinical dashboard 1300 can also be used to support auditing and explainability. The system can store the dashboard state, input features, signal-quality flags, classifier outputs, matched-cohort parameters, and generated outputs at each update time. The stored information can be used for clinical review, quality assurance, model improvement, regulatory documentation, or retrospective analysis. This can provide a technical and operational advantage because the system can record not only the final output but also the data and confidence conditions that produced the output.

The dashboard 1300 can also support adaptive monitoring by presenting system-control changes to the care team. For example, the dashboard can indicate that the system increased sampling rate, increased feature-extraction frequency, increased data-transmission frequency, extended waveform-buffer retention, changed an alert threshold, or selected a heightened monitoring mode in response to a deviation or deterioration classification. Conversely, the dashboard can indicate that monitoring intensity was reduced after a stability period. By displaying these changes, the dashboard can make the adaptive operation of the apparatus 100 visible and understandable.

FIG. 13 can therefore illustrate the final care-team-facing layer of the overall brain-injury treatment-response monitoring system 10. The clinical dashboard 1300 can bring together the signal-processing workflow of FIG. 10, the matched-cohort selection of FIG. 11, and the treatment-response classification workflow of FIG. 12. The result can be a display that presents not merely raw data or a generic trajectory, but a reliability-aware, cohort-contextualized, event-anchored treatment-response output for use during recovery from brain injury.

### Additional Exemplary Embodiments of the Invention

In some embodiments, the brain-injury treatment-response monitoring system can generate and maintain a reference dataset that is separate from the continuous EEG waveform data. The reference dataset can include analytic measures extracted from EEG waveform data, non-EEG physiological data, clinical context data, imaging data, text data, or combinations thereof. The reference dataset can be stored in the brain-state feature table, in a local memory of the bedside monitoring apparatus, in a remote cohort database, or in another computer-readable storage structure. The reference dataset can be queried to identify patterns, shifts, alignments, trends, or other content derived from the patient data without requiring direct clinician assessment of the raw EEG waveform data.

The reference dataset can include analytic measures grouped by feature type, time window, treatment event, injury type, severity class, signal-quality state, patient state, medication state, sedation state, or monitoring mode. For example, the reference dataset can include frequency-domain EEG features, time-domain EEG features, spatial EEG features, physiologic features, clinical-event features, imaging-derived features, and text-derived clinical features. The system can use the reference dataset as an intermediate data structure that allows later comparison, classification, cohort selection, and treatment-response output generation to be performed using stored analytic measures rather than repeated processing of the same raw waveform samples.

In one example, the system can identify target content within continuous EEG waveform data or other patient data. The target content can include seizure content, burst-suppression content, slowing content, asymmetry content, ischemia-associated content, hemorrhage-associated content, medication-effect content, sedation-effect content, stimulation-reactivity content, artifact content, or other characteristic content relevant to a brain-injury patient. The target content can be represented by a combination of shifts in multiple analytic measures rather than by a single feature alone.

A surrogate pattern can be generated from the analytic measures to represent the target content. The surrogate pattern can include a reproducible alignment, relationship, or co-occurrence of multiple feature shifts. For example, seizure-related content can be represented by coordinated changes across frequency power, rhythmicity, amplitude, spatial spread, entropy, synchrony, and temporal evolution. Ischemia-related content can be represented by coordinated changes across slowing, asymmetry, reduced variability, spectral-power redistribution, and clinical context. A medication-effect pattern can be represented by coordinated changes across EEG slowing, burst suppression, vital signs, medication timing, and sedation state.

The system can use one or more pattern-confirmation operations to confirm that a surrogate pattern corresponds to target content. Pattern confirmation can include statistical comparison, threshold comparison, template matching, feature-alignment analysis, classifier output comparison, cohort comparison, temporal persistence analysis, cross-channel consistency analysis, or comparison against previously confirmed patient data. In some embodiments, a reference dataset containing only or primarily target content can be used to identify a characteristic feature alignment associated with the target content. As additional data becomes available, full-length datasets containing target content and non-target content can be screened to identify similar alignments of features.

The electronic processor can query the reference dataset to identify characteristic content represented by a surrogate pattern. The query can be initiated automatically, periodically, in response to a treatment event, in response to a detected feature shift, in response to a clinician request, in response to a signal-quality change, or in response to a monitoring-mode change. The query can identify whether the patient data includes a pattern associated with improvement, deterioration, plateau, deviation, seizure activity, ischemic evolution, hemorrhagic evolution, medication effect, sedation effect, artifact, or another clinically relevant state.

In some embodiments, the system can use the surrogate pattern as an input to the treatment-response engine. For example, a pre-event surrogate pattern can be generated from analytic measures associated with a pre-event temporal window, and a post-event surrogate pattern can be generated from analytic measures associated with a post-event temporal window. The treatment-response engine can compare the pre-event surrogate pattern to the post-event surrogate pattern to determine whether the treatment event is associated with a favorable pattern shift, unfavorable pattern shift, unchanged pattern, unexpected deviation, or indeterminate result.

The analytic measures can include manually designed features, machine-generated features, learned features, statistical features, or combinations thereof. EEG-derived analytic measures can include alpha-to-delta ratio, delta power, theta power, alpha power, beta power, gamma power, peak frequency, spectral edge frequency, power in frequency bands, symmetry measures, interhemispheric asymmetry, temporal latency corresponding to a stimulus event, auto-regressive coefficients, wavelet coefficients, coherence, connectivity, entropy, burst-suppression burden, seizure burden, rhythmicity, amplitude variability, stimulation reactivity, or other waveform-derived features.

The system can also use analytic measures derived from non-EEG physiological signals. For example, ECG data can be processed to generate heart-rate, rhythm, heart-rate-variability, or cardiac-event features. Intracranial-pressure data can be processed to generate pressure values, pressure trends, pulse-amplitude features, plateau-wave indicators, or pressure-reactivity features. Arterial-pressure data, oxygenation data, respiratory data, temperature data, NIRS data, movement data, and other physiological data can be processed to generate additional features for evaluating patient state, signal reliability, treatment response, or confidence.

In some embodiments, the system can receive clinical information from multiple clinical sources. The clinical information can include physician notes, nursing notes, medication records, procedure records, neurologic examination findings, imaging reports, laboratory values, diagnosis codes, stroke scales, Glasgow Coma Scale values, discharge summaries, rehabilitation notes, and outcome information. The system can process text-format clinical information using natural language processing to extract structured features, such as treatment-event type, treatment-event time, medication dose, sedation state, injury mechanism, symptom onset, clinical deterioration, or outcome anchor.

The imaging data used by the system can include CT data, MRI data, ultrasound data, transcranial Doppler data, perfusion data, angiographic data, or other imaging information. The imaging data can be processed using image processing, computer vision, segmentation, feature extraction, radiographic-score extraction, vascular-territory identification, hemorrhage-volume estimation, infarct-volume estimation, midline-shift estimation, edema estimation, blood-flow estimation, or other imaging-analysis techniques. Imaging-derived features can be provided to an imaging classifier or used as patient-matching parameters for selecting a matched brain-injury cohort.

The system can include a plurality of modality-specific classifiers. A text data classifier can evaluate features derived from text-format clinical information. An imaging data classifier can evaluate features derived from imaging data. A biosignal data classifier can evaluate features derived from EEG data, ECG data, ICP data, arterial-pressure data, NIRS data, or other physiological signals. Each classifier can generate a probability, score, label, severity class, recovery class, treatment-response class, or confidence value associated with the patient.

The outputs of the modality-specific classifiers can be combined using decision fusion. Decision fusion can include averaging, weighted averaging, voting, confidence-weighted voting, Bayesian fusion, ensemble modeling, stacking, rule-based fusion, time-windowed fusion, or another fusion technique. The weighting of a classifier output can depend on signal quality, data availability, data recency, missing-data status, injury type, treatment-event type, cohort-match quality, medication state, sedation state, or clinical context. For example, an EEG classifier output can be down-weighted when EEG signal quality is poor, and an imaging classifier output can be down-weighted when the imaging study is temporally remote from the treatment event.

In one embodiment, each classifier can output a relative probability for each of a plurality of severity classes. The severity classes can represent different levels of brain injury severity, recovery state, treatment-response state, or predicted recovery potential. The decision fusion can combine the classifier probabilities to produce a fused probability distribution. The fused probability distribution can be used to determine a current severity estimation, a recovery trajectory, a treatment-response state, a confidence indicator, or a predicted outcome.

The plurality of severity classes can include any suitable number of classes. In one example, five severity classes can be used, with a lower class indicating a more favorable neurological state and a higher class indicating a less favorable neurological state. In other embodiments, the classes can be binary, ordinal, continuous, probabilistic, percentile-based, or tied to clinically meaningful outcome categories. The system can update the class probabilities as new EEG data, physiological data, imaging data, text data, treatment-event data, or outcome data becomes available.

The system can update different data streams at different rates. Biosignal data, including EEG data, can be updated every few seconds, every few minutes, or according to another high-frequency acquisition schedule. Text data, including progress notes and medication records, can be updated when new documentation or medication-administration information becomes available. Imaging data can be updated when a new imaging study is obtained or when an existing imaging study is reprocessed. The system can align these heterogeneous update rates using timestamps, event anchors, temporal windows, data freshness indicators, and confidence values.

A recovery trajectory can be calculated and updated based on analytic measures, surrogate patterns, classifier outputs, historical data, matched-cohort data, or combinations thereof. The recovery trajectory can indicate changes in a severity of the brain injury over time, changes in recovery potential, changes in treatment response, or changes relative to a matched cohort. The recovery trajectory can be updated in response to new clinical information, new raw biometric data, new analytic measures, new treatment-event data, new cohort-response data, new classifier output, or a change in signal-quality status.

The display can present the recovery trajectory graphically. In one embodiment, the graph can be divided into a plurality of regions representing different recovery conditions. For example, a first region can indicate poor recovery conditions, a second region can indicate average recovery conditions, and a third region can indicate excellent recovery conditions. The regions can be computationally determined based on severity classifications, analytic measures, reference dataset values, matched-cohort data, normative modeled recovery data, or outcome anchors. The patient's recovery trajectory can be plotted relative to the regions as analytic measures are updated over time.

The displayed regions can also be event-anchored. For example, the graph can show a patient response before and after a treatment event relative to poor, average, or excellent expected response regions generated from a matched brain-injury cohort. A patient response line can be displayed relative to one or more boundaries, corridors, percentile bands, or recovery regions. A deviation flag can be generated when the patient response exits an expected region, crosses a boundary, changes region classification, or moves in an unexpected direction after the treatment event.

The system can determine a predicted recovery outcome based on comparison of the patient's analytic measures and clinical information to historical data for other patients. The historical data can include prior patient EEG features, treatment-event histories, clinical context, imaging findings, text-derived features, physiological trends, outcome anchors, and recovery trajectories. The predicted recovery outcome can include a probability of functional recovery, probability of deterioration, probability of improvement, expected discharge status, rehabilitation potential, expected neurological state, expected recovery region, or other outcome-related information.

In some embodiments, the system can identify a trend in changes to a determined severity of the injury over a period of time. The trend can be calculated as a slope, rate of change, direction of change, classifier-probability shift, feature-space shift, region transition, percentile movement, or other temporal relationship. The trend can be calculated over minutes, hours, days, or another interval selected based on injury type, treatment-event type, monitoring mode, or clinical context. The trend can be used to update the recovery trajectory, select a monitoring mode, generate a treatment-management output, or trigger additional analysis.

The system can use baseline information when evaluating the patient. Baseline information can include a patient-specific baseline, a pre-injury baseline, an admission baseline, a pre-event baseline, a cohort baseline, a normative baseline, or a disease-subpopulation baseline. The baseline can be used to normalize analytic measures, identify deviations from a patient's own state, identify deviations from a matched cohort, or distinguish treatment-related changes from pre-existing patient variability. When a patient-specific baseline is unavailable, the system can use cohort baseline data, injury-specific baseline data, or normative data.

The system can also identify deviations from baseline, deviations from norm, and deviations from expected recovery. A deviation from baseline can compare the patient's current state to the patient's own earlier state. A deviation from norm can compare the patient's current state to a normative population or reference group. A deviation from expected recovery can compare the patient's current state or treatment response to a matched cohort, expected recovery corridor, or expected intervention-response envelope. These different deviation types can be displayed separately or fused into a treatment-response output.

In one example, a care-team output can display a current severity estimate, a predicted recovery trajectory, a treatment-response classification, and a confidence indicator. The output can also display the underlying classifier probabilities, the data streams used to generate the output, the current biosignals used by the model, and the calculated parameters supporting the result. This can allow a clinician to review both the high-level recovery output and the intermediate data used to produce that output.

The system can store calculated parameters in a table for later display, export, audit, classifier input, or cohort comparison. The calculated-parameter table can include feature names, values, indices, ratios, mean values, mode values, kurtosis values, high/normal/low classifications, temporal-window identifiers, event anchors, signal-quality flags, and source identifiers. The calculated-parameter table can allow rapid identification of features within analyzed biosignals and can provide a traceable link between raw patient data and generated recovery or treatment-response outputs.

A current-biosignal display can identify which biosignals are currently being used by the system. The current-biosignal display can include EEG, ECG, ICP, arterial-line data, cuff pressure data, pharmacy data, recent lab values, NIRS data, respiratory data, temperature data, movement data, or other patient data. The display can indicate whether each signal is active, missing, unreliable, excluded, down-weighted, delayed, or used with reduced confidence. This can help explain why a recovery trajectory or treatment-response state was generated, delayed, qualified, or marked indeterminate.

The system can support training, updating, and validating one or more classifiers. Training data can include prior patient data labeled according to severity class, recovery state, treatment-response state, outcome, or expert annotation. The training data can include reference datasets containing target patterns, full-length datasets containing both target content and non-target content, matched-cohort datasets, or de-identified multi-institutional datasets. Classifier training can be performed locally, remotely, periodically, continuously, or offline. Updated models or model parameters can be transmitted to the bedside monitoring apparatus.

The classifier can be trained to recognize changes in brain injury level, recovery potential, treatment response, seizure burden, ischemic evolution, hemorrhagic evolution, medication effect, sedation effect, artifact, or other patient states. The classifier can use supervised learning, semi-supervised learning, unsupervised learning, transfer learning, deep learning, ensemble learning, regression, classification, clustering, dimensionality reduction, or other machine-learning techniques. The classifier output can be used alone or fused with other classifier outputs to generate the final decision output.

Some embodiments can include missing-data handling. When one or more data streams are unavailable, delayed, incomplete, or unreliable, the system can omit the missing data, impute missing values, down-weight the affected data stream, select an alternate classifier, alter a confidence value, request additional data, adjust a monitoring mode, or classify the result as indeterminate. Missing-data handling can be particularly useful when imaging data is intermittent, clinical notes are delayed, EEG channels are missing, or physiological sensors are disconnected.

The system can also perform artifact handling. Artifact handling can include detecting, flagging, rejecting, correcting, filtering, down-weighting, or segmenting artifact-contaminated data. Artifact sources can include muscle activity, motion, electrode displacement, poor electrode contact, environmental noise, stimulation artifact, procedure-related artifact, cardiac artifact, respiratory artifact, or data dropout. The artifact handling can be performed before feature extraction, after feature extraction, during signal-quality gating, during classifier weighting, or during output generation.

In some embodiments, the system can receive stimulus-event data and evaluate temporal latency corresponding to the stimulus event. A stimulus event can include auditory stimulation, visual stimulation, tactile stimulation, electrical stimulation, neurologic examination stimulation, rehabilitation stimulation, or another intentional or incidental stimulus. The system can extract latency features, amplitude features, reactivity features, evoked-response features, or recovery-of-reactivity features from EEG waveform data. These features can be used as analytic measures in the brain-state feature table and can contribute to recovery-state or treatment-response classification.

The system can generate reports, exports, or data packages for clinical review, research, regulatory documentation, or model improvement. A report can include treatment events, temporal windows, feature vectors, surrogate patterns, classifier outputs, probability distributions, recovery trajectories, signal-quality flags, confidence indicators, matched-cohort parameters, and treatment-management outputs. The report can be stored locally, transmitted to an electronic health record, transmitted to a remote cohort server, or made available through an application programming interface.

The system can be configured so that raw waveform data, feature vectors, compressed brain-state signatures, classifier outputs, and treatment-management outputs have different retention policies. For example, raw EEG waveform data can be retained locally for a first period, compressed brain-state signatures can be retained for a second period, and generated treatment-response outputs can be retained in an EHR for a third period. The retention policy can be based on storage capacity, clinical need, privacy requirements, monitoring mode, signal-quality state, treatment-event type, or user configuration.

In use, the system can receive continuous EEG waveform data and clinical information, transform the EEG waveform data into analytic measures, categorize the analytic measures into a reference dataset, query the reference dataset to identify target content represented by one or more surrogate patterns, and calculate or update a recovery trajectory or treatment-response output based on the surrogate patterns and comparative data from prior brain-injury patients. The output can be displayed without requiring direct assessment of the continuous EEG waveform data, while still allowing the raw waveform data or selected waveform excerpts to be reviewed when desired.

The additional embodiments described in this section can be used alone or in combination with the event-anchored treatment-response embodiments described above. For example, surrogate-pattern recognition can be used to generate a treatment-response vector, modality-specific classifier probabilities can be used to select or weight a matched brain-injury cohort, severity-class probability outputs can be used to define display regions, and reference-dataset queries can be used to reduce repeated processing of raw EEG waveform data. These combinations can provide additional technical support for a brain-injury treatment-response monitoring system that transforms complex patient data into structured recovery and treatment-response outputs.

In some embodiments, a brain-injury treatment-response monitoring system 10 can be configured for managing recovery of a patient during a treatment period following a brain injury. The system 10 can include a neurophysiological sensor interface, which can be implemented by or through communication interface 108, a clinical-event interface, which can be implemented by or through clinical-event input 120 and/or communication interface 108, a memory 106, an output interface 124, and one or more processors 104 coupled to the neurophysiological sensor interface, the clinical-event interface, the memory 106, and the output interface 124. The neurophysiological sensor interface can receive continuous electroencephalogram (EEG) waveform data from at least one EEG sensor 116 coupled to the patient. The clinical-event interface can receive time-stamped clinical-event data identifying a treatment event 144 performed during the treatment period.

The memory 106 can store a rolling waveform buffer 126, a brain-state feature table 112, and matched-cohort recovery data 128 for prior brain-injury patients. The rolling waveform buffer 126 can store continuous EEG waveform data received from at least one EEG sensor 116. The brain-state feature table 112 can store feature vectors, analytic measures, time-window identifiers, event-time information, signal-quality values, and other metadata derived from the continuous EEG waveform data. The matched-cohort recovery data 128 can include recovery information, treatment-response information, outcome information, injury-severity information, clinical-event information, EEG-derived feature information, or other data associated with prior brain-injury patients.

The one or more processors 104 can receive the continuous EEG waveform data into the rolling waveform buffer 126 and can time-align the treatment event 144 with the continuous EEG waveform data using an event time associated with the treatment event 144. The event time can correspond to a start time, stop time, administration time, procedure time, onset time, documentation time, detection time, or estimated time associated with the treatment event 144. The one or more processors 104 can define a pre-event temporal window 140 and a post-event temporal window 142 relative to the event time.

EEG waveform samples in the pre-event temporal window 140 can be transformed into a pre-event brain-state feature vector 132 stored in the brain-state feature table 112. EEG waveform samples in the post-event temporal window 142 can be transformed into a post-event brain-state feature vector 134 stored in the brain-state feature table 112. The pre-event brain-state feature vector 132 and the post-event brain-state feature vector 134 can each include at least two EEG-derived features, including spectral power, interhemispheric asymmetry, burst-suppression burden, seizure burden, coherence, entropy, rhythmicity, amplitude variability, stimulation reactivity, or combinations thereof.

The system 10 can apply a signal-quality gate 114 to the pre-event brain-state feature vector 132 and the post-event brain-state feature vector 134. The signal-quality gate 114 can determine whether either feature vector 132, 134 fails a reliability threshold 136. The reliability threshold 136 can be based on one or more of an electrode impedance value, a missing-channel condition, a signal saturation condition, a motion artifact condition, a muscle artifact condition, a sedative-confounder condition, a medication-confounder condition, a data-dropout condition, or another signal-quality condition. When the signal-quality gate 114 determines that either the pre-event brain-state feature vector 132 or the post-event brain-state feature vector 134 fails the reliability threshold 136, the one or more processors 104 can prevent classification of a treatment-response state.

The one or more processors 104 can select, from the matched-cohort recovery data 128, a matched brain-injury cohort 1122 based on patient-matching parameters 1118. The patient-matching parameters 1118 can include an injury type of the brain injury, an initial injury severity, a time from an injury onset, and a treatment-event type of the treatment event 144. The patient-matching parameters 1118 can further include an age of the patient, an imaging finding of the patient, a vascular territory of the brain injury, a Glasgow Coma Scale score, a National Institutes of Health Stroke Scale score, an intracranial pressure value, a medication state, a sedation state, a neurological examination finding, or combinations thereof.

The treatment-event type can include administration of an anti-seizure medication, administration of a sedative medication, administration of an osmotic therapy, a blood-pressure intervention, a ventilation adjustment, an intracranial-pressure intervention, a thrombolytic intervention, a thrombectomy intervention, a rehabilitation intervention, a neurological stimulation event, or another treatment event 144 performed during the treatment period. The system 10 can use the treatment-event type to select the matched brain-injury cohort 1122 so that the patient response is compared against an event-specific expected response rather than a generic recovery trend.

The one or more processors 104 can generate an expected intervention-response envelope 910 for the treatment event 144 based on the matched brain-injury cohort 1122. The expected intervention-response envelope 910 can include a plurality of time-indexed percentile boundaries 138 generated from the matched brain-injury cohort 1122. The percentile boundaries 138 can define an expected range, upper and lower response boundaries, a recovery corridor, a deviation boundary, or another cohort-derived comparative structure. The one or more processors 104 can compare a treatment-response vector 1204 to the plurality of time-indexed percentile boundaries 138.

A treatment-response vector 1204 can be generated to represent a change from the pre-event brain-state feature vector 132 to the post-event brain-state feature vector 134. The treatment-response vector 1204 can include differences, ratios, normalized differences, feature-space distances, direction-of-change values, classifier-probability shifts, or other values indicating how the patient's brain state changed across the treatment event 144. The treatment-response vector 1204 can be compared to the expected intervention-response envelope 910 to determine whether the patient's response is within, above, below, or outside the expected response for the matched brain-injury cohort 1122.

Based on the comparison, the one or more processors 104 can classify the treatment-response state of the patient as one of improvement, deterioration, plateau, deviation, or indeterminate. The classification can be generated only after the signal-quality gate 114 determines that the feature vectors 132, 134 satisfy the reliability threshold 136. If the data is insufficiently reliable, the treatment-response state can be classified as indeterminate or classification can be suppressed, delayed, or qualified.

The output interface 124 can generate a treatment-management output 130 identifying the treatment event 144, the treatment-response state, and a confidence indicator 1308. The treatment-management output 130 can include an escalation indication, a de-escalation indication, a monitoring-frequency change indication, an intervention-effectiveness indication, an alert indicating deviation from expected recovery, a dashboard output 1300, a report, a notification, or another care-team-facing output. The confidence indicator 1308 can reflect signal quality, cohort-match quality, data completeness, model confidence, clinical-context reliability, or other confidence information.

The comparison between the treatment-response vector 1204 and the expected intervention-response envelope 910 can be performed using the pre-event brain-state feature vector 132 and the post-event brain-state feature vector 134 stored in the brain-state feature table 112. In some embodiments, the comparison can be performed without reprocessing the EEG waveform samples in the rolling waveform buffer 126 after the pre-event brain-state feature vector 132 and the post-event brain-state feature vector 134 are stored in the brain-state feature table 112. This allows the system 10 to use a stored feature-based representation for comparison and classification, which can reduce repeated raw-waveform processing and improve the operation of the monitoring system 10.

A method of using the system 10 can include coupling the at least one EEG sensor 116 to the patient during the treatment period, receiving the continuous EEG waveform data from the at least one EEG sensor 116 through the neurophysiological sensor interface, receiving the time-stamped clinical-event data identifying the treatment event 144 through the clinical-event interface, generating the pre-event brain-state feature vector 132 and the post-event brain-state feature vector 134, applying the signal-quality gate 114 to the feature vectors 132, 134, classifying the treatment-response state of the patient, and generating the treatment-management output 130 through the output interface 124.

In another exemplary embodiment, a brain-injury treatment-response monitoring system 10 can include a neurophysiological sensor interface, a physiological sensor interface 146, a clinical-event interface, a clinical data interface 148, a memory 106 storing a monitoring protocol table 150, and one or more processors 104. The neurophysiological sensor interface can receive continuous EEG waveform data from at least one EEG sensor 116 coupled to the patient. The physiological sensor interface 146 can receive non-EEG physiological data from at least one non-EEG physiological sensor coupled to the patient. The clinical-event interface can receive time-stamped clinical-event data identifying a treatment event 144 performed during the treatment period. The clinical data interface 148 can receive clinical context data associated with the patient.

The non-EEG physiological data can include intracranial pressure data, arterial pressure data, electrocardiogram data, pulse oximetry data, respiratory data, temperature data, movement data, NIRS data, or other physiological data. The clinical context data can include injury type, time from injury onset, neurological examination findings, imaging findings, medication state, sedation state, laboratory values, demographic information, clinical-history information, treatment-event history, outcome information, or other patient-context information.

The monitoring protocol table 150 can associate a plurality of monitoring modes 152 with different acquisition parameters 154 and different processing parameters 156. The acquisition parameters 154 can include sampling rate, EEG channel set, electrode montage, data-transmission frequency, waveform-buffer retention period, sensor-selection setting, acquisition duration, impedance-check frequency, or another parameter affecting acquisition or receipt of data. The processing parameters 156 can include feature-extraction interval, classifier-update interval, alert threshold, signal-quality threshold, cohort-comparison interval, artifact-rejection setting, temporal-window length, confidence threshold, model-selection setting, or another parameter affecting processing of patient data.

The one or more processors 104 can generate a current brain-state vector 158 from the continuous EEG waveform data over a current temporal window and can generate a previous brain-state vector 160 from the continuous EEG waveform data over a previous temporal window. The current temporal window and the previous temporal window can be adjacent, overlapping, separated, event-defined, clinician-selected, protocol-selected, or automatically selected. The current brain-state vector 158 and the previous brain-state vector 160 can include EEG-derived features and can optionally include additional physiological or clinical-context features.

A recovery-trend value 162 can be generated based on a change from the previous brain-state vector 160 to the current brain-state vector 158. The recovery-trend value 162 can indicate whether the patient is improving, deteriorating, plateauing, deviating, stabilizing, or otherwise changing over time. The recovery-trend value 162 can be calculated as a difference, slope, direction, distance, probability shift, percentile movement, classifier-output change, feature-space change, or another value representing a change in brain-state information.

The one or more processors 104 can generate a confidence value 164 based on the non-EEG physiological data and the clinical context data. The confidence value 164 can reflect whether the EEG-derived brain-state information is consistent with other physiological information, whether clinical context is sufficient, whether sedation or medication state confounds interpretation, whether non-EEG sensors are active or reliable, whether data streams agree with one another, or whether the patient data is otherwise reliable for adaptive monitoring.

The recovery-trend value 162 can be compared to an expected recovery corridor 166 for the brain injury. The expected recovery corridor 166 can be generated from matched-cohort data, injury-specific recovery data, patient-specific baseline data, normative data, or model-generated recovery data. The expected recovery corridor 166 can include boundaries, percentile bands, confidence intervals, expected trend lines, time-indexed ranges, or other comparative information.

The system 10 can select a selected monitoring mode 168 from the plurality of monitoring modes 152 based on the comparison between the recovery-trend value 162 and the expected recovery corridor 166, the confidence value 164, and a treatment-event type of the treatment event 144. For example, the system 10 can select a heightened-monitoring mode when the patient is deviating from the expected recovery corridor 166 with sufficient confidence. The system 10 can select a reduced-monitoring mode when the patient remains within the expected recovery corridor 166 for a stability period. Different treatment-event types can have different monitoring modes 152, thresholds, temporal windows, or processing requirements.

A heightened-monitoring mode can be selected only when the recovery-trend value 162 satisfies a deviation criterion 170 for at least two consecutive temporal windows and the confidence value 164 satisfies a confidence threshold 172. This can reduce unnecessary monitoring-mode changes based on transient artifact, noise, or a single unreliable window. The deviation criterion 170 can include crossing a boundary, exceeding a percentile threshold, moving outside the expected recovery corridor 166, worsening by a threshold amount, or satisfying another deviation condition. The confidence threshold 172 can require sufficient signal quality, clinical-context availability, non-EEG physiological consistency, or cohort-match quality.

A selected monitoring mode 168 can reduce at least one of a sampling rate, a feature-extraction frequency, or a data-transmission frequency when the recovery-trend value 162 remains within the expected recovery corridor 166 for a stability period 174. The stability period 174 can be measured in minutes, hours, days, monitoring windows, event windows, or another interval. The reduced-monitoring mode can preserve computing resources, reduce network bandwidth, reduce storage burden, reduce alarm burden, or extend battery life while continuing to monitor the patient.

The one or more processors 104 can generate a control command 176 corresponding to the selected monitoring mode 168. The control command 176 can specify a change to at least one acquisition parameter 154 of the neurophysiological sensor interface or at least one processing parameter 156 used by the one or more processors 104. The control command 176 can change operation of the brain-injury treatment-response monitoring system 10 during the treatment period. The control command 176 can be applied before a subsequent brain-state vector 178 is generated so that the subsequent brain-state vector 178 is generated under the selected monitoring mode 168.

After operation of the monitoring system 10 has been changed according to the control command 176, the system 10 can generate a treatment-response output 180. The treatment-response output 180 can identify the selected monitoring mode 168, the recovery-trend value 162, the confidence value 164, the treatment-event type, the expected recovery corridor 166, the monitoring-control change, or the resulting treatment-response state. In this manner, the system 10 can adapt its own acquisition or processing operation during brain-injury recovery rather than merely displaying patient information.

A method of using this adaptive monitoring system 10 can include receiving continuous EEG waveform data through the neurophysiological sensor interface, receiving non-EEG physiological data through the physiological sensor interface 146, receiving time-stamped clinical-event data through the clinical-event interface, generating the recovery-trend value 162, selecting the selected monitoring mode 168 from the plurality of monitoring modes 152, applying the control command 176 to change operation of the brain-injury treatment-response monitoring system 10, and generating the treatment-response output 180 after operation of the monitoring system 10 has been changed.

In another implementation, the brain-injury treatment-response monitoring system 10 can include a bedside monitoring apparatus 100 and a remote cohort server 118. The bedside monitoring apparatus 100 can include a neurophysiological sensor interface configured to receive continuous EEG waveform data from at least one EEG sensor 116 coupled to the patient, a local memory 106 configured to store the continuous EEG waveform data, and a local output interface 124. The remote cohort server 118 can include a cohort database storing recovery data for prior brain-injury patients. A network interface 108 can communicatively couple the bedside monitoring apparatus 100 to the remote cohort server 118.

The bedside monitoring apparatus 100 can include one or more local processors 104, and the remote cohort server 118 can include one or more server processors 182. The one or more local processors 104 can transform the continuous EEG waveform data into a compressed brain-state signature 184. The compressed brain-state signature 184 can include a time-indexed sequence of EEG-derived feature vectors 186. The compressed brain-state signature 184 can further include signal-quality flags 188, artifact flags 190, medication-state flags 192, sedation-state flags 194, treatment-event metadata 196, cohort-matching parameters, confidence values, temporal-window identifiers, or other metadata.

The one or more local processors 104 can associate the compressed brain-state signature 184 with treatment-event metadata 196 identifying a treatment event 144 performed during the treatment period. The treatment-event metadata 196 can include event type, event time, event duration, event dose, event source, event confidence, clinical-system source, or other metadata associated with the treatment event 144. The local processors 104 can transmit, through the network interface 108, the compressed brain-state signature 184 and the treatment-event metadata 196 to the remote cohort server 118 without transmitting the continuous EEG waveform data to the remote cohort server 118.

In some embodiments, the one or more local processors 104 can remove direct patient identifiers 198 from the compressed brain-state signature 184 before transmitting the compressed brain-state signature 184 to the remote cohort server 118. Direct patient identifiers 198 can include patient name, medical-record number, date of birth, address, direct contact information, or other direct identifiers. The compressed brain-state signature 184 can retain sufficient clinical and feature-based information to support cohort matching while reducing transmission of identifying information and raw waveform data.

The one or more server processors 182 can select, from the cohort database of the remote cohort server 118, a matched brain-injury cohort 1122 based on the compressed brain-state signature 184 and the treatment-event metadata 196. The cohort database can store recovery data, treatment-response data, EEG-derived feature data, clinical-event data, outcome data, imaging data, text-derived data, and other information for prior brain-injury patients. The matched brain-injury cohort 1122 can be selected using similarity scores 1120, inclusion/exclusion rules 1110, ranked candidate cases, weighted matching, or other cohort-selection logic.

The remote cohort server 118 can generate cohort-response data 199 from the matched brain-injury cohort 1122. The cohort-response data 199 can include an expected response envelope 910, expected recovery corridor 166, percentile boundaries 138, outcome probabilities, treatment-response distributions, cohort summary statistics, similarity scores 1120, confidence indicators, or other response data derived from the matched brain-injury cohort 1122. The remote cohort server 118 can transmit the cohort-response data 199 to the bedside monitoring apparatus 100 through the network interface 108.

The one or more local processors 104 of the bedside monitoring apparatus 100 can classify a treatment-response state of the patient using the cohort-response data 199 and the compressed brain-state signature 184. The treatment-response state can include improvement, deterioration, plateau, deviation, indeterminate, within expected range, above expected range, below expected range, or another response state. The local output interface 124 can generate a treatment-response output 180 identifying the treatment-response state.

The continuous EEG waveform data can remain stored locally at the bedside monitoring apparatus 100 while the compressed brain-state signature 184 is transmitted to the remote cohort server 118. In this arrangement, the remote cohort server 118 can perform cohort selection and cohort-response generation without receiving the continuous EEG waveform data. This can reduce network load, reduce remote storage requirements, improve privacy, and allow the bedside monitoring apparatus 100 to maintain local control over raw waveform data.

The local memory 106 can also store cached cohort-response data 200. When communication between the bedside monitoring apparatus 100 and the remote cohort server 118 is unavailable, delayed, interrupted, or unreliable, the one or more local processors 104 can classify the treatment-response state using the cached cohort-response data 200. The cached cohort-response data 200 can include previously received expected response envelopes 910, recovery corridors 166, percentile boundaries 138, cohort summaries, or model parameters. When communication is restored, the bedside monitoring apparatus 100 can receive updated cohort-response data 199 from the remote cohort server 118.

A method of using the distributed system 10 can include receiving continuous EEG waveform data through the neurophysiological sensor interface, storing the continuous EEG waveform data in local memory 106 at the bedside monitoring apparatus 100, transforming the continuous EEG waveform data into the compressed brain-state signature 184, transmitting the compressed brain-state signature 184 and treatment-event metadata 196 to the remote cohort server 118 without transmitting the continuous EEG waveform data, generating cohort-response data 199 using the one or more server processors 182, transmitting the cohort-response data 199 to the bedside monitoring apparatus 100, and generating the treatment-response output 180 through the local output interface 124.

The embodiments described in this section can be used individually or together. For example, a bedside monitoring apparatus 100 can use the signal-quality gate 114, pre-event and post-event brain-state feature vectors 132, 134, matched-cohort expected intervention-response envelope 910, adaptive monitoring protocol table 150, and compressed brain-state signature 184 in a single implementation. In another implementation, only selected features can be used, such as the event-anchored feature-vector comparison, the adaptive monitoring-control operation, or the local-versus-remote compressed-signature workflow. These embodiments provide express support for systems and methods that process continuous EEG waveform data, treatment-event data, patient context, matched-cohort recovery data, and monitoring-control data to generate treatment-response outputs during a treatment period following a brain injury.

The capabilities of the present invention can be implemented in software, firmware, hardware, or some combination thereof.

As one example, one or more aspects of the present invention can be included in an article of manufacture (e.g., one or more computer program products) having, for instance, computer-usable media. The media has embodied therein, for instance, computer-readable program code means for providing and facilitating the capabilities of the present invention. The article of manufacture can be included as a part of a computer system or sold separately.

Additionally, at least one program storage device readable by a machine, tangibly embodying at least one program of instructions executable by the machine to perform the capabilities of the present invention can be provided.

The flow diagrams depicted herein are just examples. There may be many variations to these diagrams or the steps (or operations) described therein without departing from the spirit of the invention. For instance, the steps may be performed in a differing order, or steps may be added, deleted, or modified. All of these variations are considered a part of the claimed invention.

While the preferred embodiment of the invention has been described, it will be understood that those skilled in the art, both now and in the future, may make various improvements and enhancements which fall within the scope of the claims which follow. These claims should be construed to maintain the proper protection for the invention first described.

What is claimed is:

1. A brain-injury treatment-response monitoring system for managing recovery of a patient during a treatment period following a brain injury, the brain-injury treatment-response monitoring system comprising:
a neurophysiological sensor interface configured to receive continuous electroencephalogram (EEG) waveform data from at least one EEG sensor coupled to the patient;
a clinical-event interface configured to receive time-stamped clinical-event data identifying a treatment event performed during the treatment period;
a memory storing a rolling waveform buffer, a brain-state feature table, and matched-cohort recovery data for prior brain-injury patients;
an output interface; and
one or more processors coupled to the neurophysiological sensor interface, the clinical-event interface, the memory, and the output interface, the one or more processors being programmed to:
receive the continuous EEG waveform data into the rolling waveform buffer;
time-align the treatment event with the continuous EEG waveform data using an event time associated with the treatment event;
define a pre-event temporal window and a post-event temporal window relative to the event time;
transform EEG waveform samples in the pre-event temporal window into a pre-event brain-state feature vector stored in the brain-state feature table;
transform EEG waveform samples in the post-event temporal window into a post-event brain-state feature vector stored in the brain-state feature table;
apply a signal-quality gate to the pre-event brain-state feature vector and the post-event brain-state feature vector;
prevent classification of a treatment-response state when the signal-quality gate determines that either the pre-event brain-state feature vector or the post-event brain-state feature vector fails a reliability threshold;
select, from the matched-cohort recovery data, a matched brain-injury cohort based on patient-matching parameters comprising an injury type of the brain injury, an initial injury severity, a time from an injury onset, and a treatment-event type of the treatment event;
generate an expected intervention-response envelope for the treatment event based on the matched brain-injury cohort;
generate a treatment-response vector representing a change from the pre-event brain-state feature vector to the post-event brain-state feature vector;
compare the treatment-response vector to the expected intervention-response envelope;
classify the treatment-response state of the patient as one of improvement, deterioration, plateau, deviation, or indeterminate based on the comparison; and
generate, via the output interface, a treatment-management output identifying the treatment event, the treatment-response state, and a confidence indicator,
wherein the one or more processors perform the comparison using the pre-event brain-state feature vector and the post-event brain-state feature vector stored in the brain-state feature table without reprocessing the EEG waveform samples in the rolling waveform buffer after the pre-event brain-state feature vector and the post-event brain-state feature vector are stored in the brain-state feature table.

2. The brain-injury treatment-response monitoring system of claim 1, wherein the pre-event brain-state feature vector and the post-event brain-state feature vector each comprise at least two EEG-derived features selected from the group consisting of spectral power, interhemispheric asymmetry, burst-suppression burden, seizure burden, coherence, entropy, rhythmicity, amplitude variability, and stimulation reactivity.

3. The brain-injury treatment-response monitoring system of claim 1, wherein the treatment-event type comprises at least one selected from the group consisting of administration of an anti-seizure medication, administration of a sedative medication, administration of an osmotic therapy, a blood-pressure intervention, a ventilation adjustment, an intracranial-pressure intervention, a thrombolytic intervention, a thrombectomy intervention, a rehabilitation intervention, and a neurological stimulation event.

4. The brain-injury treatment-response monitoring system of claim 1, wherein the signal-quality gate determines that the reliability threshold has failed based on at least one of an electrode impedance value, a missing-channel condition, a signal saturation condition, a motion artifact condition, a muscle artifact condition, or a sedative-confounder condition.

5. The brain-injury treatment-response monitoring system of claim 1, wherein the expected intervention-response envelope comprises a plurality of time-indexed percentile boundaries generated from the matched brain-injury cohort, and wherein the one or more processors are programmed to compare the treatment-response vector to the plurality of time-indexed percentile boundaries.

6. The brain-injury treatment-response monitoring system of claim 1, wherein the treatment-management output comprises at least one of an escalation indication, a de-escalation indication, a monitoring-frequency change indication, an intervention-effectiveness indication, or an alert indicating deviation from expected recovery.

7. The brain-injury treatment-response monitoring system of claim 1, wherein the patient-matching parameters further comprise at least two selected from the group consisting of an age of the patient, an imaging finding of the patient, a vascular territory of the brain injury, a Glasgow Coma Scale score, a National Institutes of Health Stroke Scale score, an intracranial pressure value, a medication state, a sedation state, and a neurological examination finding.

8. A method of using the brain-injury treatment-response monitoring system of claim 1, the method comprising the steps of:
coupling the at least one EEG sensor to the patient during the treatment period;
receiving, by the neurophysiological sensor interface, the continuous EEG waveform data from the at least one EEG sensor;
receiving, by the clinical-event interface, the time-stamped clinical-event data identifying the treatment event;
generating, by the one or more processors, the pre-event brain-state feature vector and the post-event brain-state feature vector;
applying, by the one or more processors, the signal-quality gate to the pre-event brain-state feature vector and the post-event brain-state feature vector;
classifying, by the one or more processors, the treatment-response state of the patient; and
generating, by the output interface, the treatment-management output.

9. A brain-injury treatment-response monitoring system for managing recovery of a patient during a treatment period following a brain injury, the brain-injury treatment-response monitoring system comprising:
a neurophysiological sensor interface configured to receive continuous electroencephalogram (EEG) waveform data from at least one EEG sensor coupled to the patient;
a physiological sensor interface configured to receive non-EEG physiological data from at least one non-EEG physiological sensor coupled to the patient;
a clinical-event interface configured to receive time-stamped clinical-event data identifying a treatment event performed during the treatment period;
a clinical data interface configured to receive clinical context data associated with the patient;
a memory storing a monitoring protocol table, the monitoring protocol table associating a plurality of monitoring modes with different acquisition parameters and different processing parameters; and
one or more processors coupled to the neurophysiological sensor interface, the physiological sensor interface, the clinical-event interface, the clinical data interface, and the memory, the one or more processors being programmed to:
generate a current brain-state vector from the continuous EEG waveform data over a current temporal window;
generate a previous brain-state vector from the continuous EEG waveform data over a previous temporal window;
generate a recovery-trend value based on a change from the previous brain-state vector to the current brain-state vector;
generate a confidence value based on the non-EEG physiological data and the clinical context data;
compare the recovery-trend value to an expected recovery corridor for the brain injury;
select a selected monitoring mode from the plurality of monitoring modes based on the comparison, the confidence value, and a treatment-event type of the treatment event;
generate a control command corresponding to the selected monitoring mode, the control command specifying a change to at least one acquisition parameter of the neurophysiological sensor interface or at least one processing parameter used by the one or more processors;
apply the control command to change operation of the brain-injury treatment-response monitoring system during the treatment period; and
generate a treatment-response output after operation of the brain-injury treatment-response monitoring system has been changed according to the control command,
wherein the control command changes operation of the brain-injury treatment-response monitoring system before a subsequent brain-state vector is generated.

10. The brain-injury treatment-response monitoring system of claim 9, wherein the at least one acquisition parameter comprises at least one selected from the group consisting of a sampling rate, an EEG channel set, an electrode montage, a data-transmission frequency, and a waveform-buffer retention period.

11. The brain-injury treatment-response monitoring system of claim 9, wherein the at least one processing parameter comprises at least one selected from the group consisting of a feature-extraction interval, a classifier-update interval, an alert threshold, a signal-quality threshold, and a cohort-comparison interval.

12. The brain-injury treatment-response monitoring system of claim 9, wherein the non-EEG physiological data comprises at least one selected from the group consisting of intracranial pressure data, arterial pressure data, electrocardiogram data, pulse oximetry data, respiratory data, temperature data, and movement data.

13. The brain-injury treatment-response monitoring system of claim 9, wherein the one or more processors are programmed to select a heightened-monitoring mode of the plurality of monitoring modes only when the recovery-trend value satisfies a deviation criterion for at least two consecutive temporal windows and the confidence value satisfies a confidence threshold.

14. The brain-injury treatment-response monitoring system of claim 9, wherein the selected monitoring mode reduces at least one of a sampling rate, a feature-extraction frequency, or a data-transmission frequency when the recovery-trend value remains within the expected recovery corridor for a stability period.

15. A method of using the brain-injury treatment-response monitoring system of claim 9, the method comprising the steps of:
receiving, by the neurophysiological sensor interface, the continuous EEG waveform data from the at least one EEG sensor;
receiving, by the physiological sensor interface, the non-EEG physiological data from the at least one non-EEG physiological sensor;
receiving, by the clinical-event interface, the time-stamped clinical-event data identifying the treatment event;
generating, by the one or more processors, the recovery-trend value;
selecting, by the one or more processors, the selected monitoring mode from the plurality of monitoring modes;
applying, by the one or more processors, the control command to change operation of the brain-injury treatment-response monitoring system; and
generating, by the one or more processors, the treatment-response output after operation of the brain-injury treatment-response monitoring system has been changed.

16. A brain-injury treatment-response monitoring system for managing recovery of a patient during a treatment period following a brain injury, the brain-injury treatment-response monitoring system comprising:
a bedside monitoring apparatus comprising:
a neurophysiological sensor interface configured to receive continuous electroencephalogram (EEG) waveform data from at least one EEG sensor coupled to the patient;
a local memory configured to store the continuous EEG waveform data; and
a local output interface;
a remote cohort server comprising a cohort database storing recovery data for prior brain-injury patients;
a network interface configured to communicatively couple the bedside monitoring apparatus to the remote cohort server;
one or more local processors of the bedside monitoring apparatus; and
one or more server processors of the remote cohort server,
wherein the one or more local processors are programmed to:
transform the continuous EEG waveform data into a compressed brain-state signature comprising a time-indexed sequence of EEG-derived feature vectors;
associate the compressed brain-state signature with treatment-event metadata identifying a treatment event performed during the treatment period;
transmit, via the network interface, the compressed brain-state signature and the treatment-event metadata to the remote cohort server without transmitting the continuous EEG waveform data to the remote cohort server; and
wherein the one or more server processors are programmed to:
select, from the cohort database, a matched brain-injury cohort based on the compressed brain-state signature and the treatment-event metadata;
generate cohort-response data from the matched brain-injury cohort; and
transmit the cohort-response data to the bedside monitoring apparatus,
wherein the one or more local processors are further programmed to:
classify a treatment-response state of the patient using the cohort-response data and the compressed brain-state signature; and
generate, via the local output interface, a treatment-response output identifying the treatment-response state,
wherein the continuous EEG waveform data remains stored locally at the bedside monitoring apparatus while the compressed brain-state signature is transmitted to the remote cohort server.

17. The brain-injury treatment-response monitoring system of claim 16, wherein the compressed brain-state signature further comprises signal-quality flags, artifact flags, medication-state flags, and sedation-state flags.

18. The brain-injury treatment-response monitoring system of claim 16, wherein the one or more local processors are programmed to remove direct patient identifiers from the compressed brain-state signature before transmitting the compressed brain-state signature to the remote cohort server.

19. The brain-injury treatment-response monitoring system of claim 16, wherein the local memory stores cached cohort-response data, and wherein the one or more local processors are programmed to classify the treatment-response state using the cached cohort-response data when communication between the bedside monitoring apparatus and the remote cohort server is unavailable.

20. A method of using the brain-injury treatment-response monitoring system of claim 16, the method comprising the steps of:
receiving, by the neurophysiological sensor interface, the continuous EEG waveform data from the at least one EEG sensor;
storing, by the local memory, the continuous EEG waveform data at the bedside monitoring apparatus;
transforming, by the one or more local processors, the continuous EEG waveform data into the compressed brain-state signature;
transmitting, by the network interface, the compressed brain-state signature and the treatment-event metadata to the remote cohort server without transmitting the continuous EEG waveform data to the remote cohort server;
generating, by the one or more server processors, the cohort-response data;
transmitting, by the remote cohort server, the cohort-response data to the bedside monitoring apparatus; and
generating, by the local output interface, the treatment-response output.
